SEQUENCE LISTING (65 sequences)

SEQ ID NO: 1 - DNA, 6 bp, Artificial Sequence
  Synthetic
cacaca

SEQ ID NO: 2 - DNA, 6 bp, Artificial Sequence
  Synthetic
acacac

SEQ ID NO: 3 - RNA, 6 nt, Artificial Sequence
  Synthetic
uucuuc

SEQ ID NO: 4 - DNA, 6 bp, Artificial Sequence
  Synthetic
ttcttc

SEQ ID NO: 5 - RNA, 6 nt, Artificial Sequence
  Synthetic
cuucuu

SEQ ID NO: 6 - DNA, 6 bp, Artificial Sequence
  Synthetic
cttctt

SEQ ID NO: 7 - RNA, 12 nt, Artificial Sequence
  Synthetic
agcuaccuagcu

SEQ ID NO: 8 - DNA, 12 bp, Artificial Sequence
  Synthetic
agctacctagct

SEQ ID NO: 9 - RNA, 12 nt, Artificial Sequence
  Synthetic
ucgauggaucga

SEQ ID NO: 10 - DNA, 12 bp, Artificial Sequence
  Synthetic
tcgatggatcga

SEQ ID NO: 11 - DNA, 21 bp, Artificial Sequence
  Synthetic
ttgagagaagtccaccacgag

SEQ ID NO: 12 - RNA, 21 nt, Artificial Sequence
  Synthetic
uugagagaaguccaccacgag

SEQ ID NO: 13 - DNA, 28 bp, Artificial Sequence
  Synthetic
cgcgggtaccaacggtgcattggaacgc

SEQ ID NO: 14 - DNA, 38 bp, Artificial Sequence
  Synthetic
atcggctagcggacggtgactgcagaaaagacccatgg

SEQ ID NO: 15 - DNA, 31 bp, Artificial Sequence
  Synthetic
atgcatgccgtgttgacaattaatcatcggc

SEQ ID NO: 16 - DNA, 30 bp, Artificial Sequence
  Synthetic
atgttaaccacgtgtcagtcctgctcctcg

SEQ ID NO: 17 - DNA, 30 bp, Artificial Sequence
  Synthetic
agccggtaccctattccagaagtagtgagg

SEQ ID NO: 18 - DNA, 30 bp, Artificial Sequence
  Synthetic
cgtaactcgagcactgcattctagttgtgg

SEQ ID NO: 19 - DNA, 30 bp, Artificial Sequence
  Synthetic
agccgctagcctattccagaagtagtgagg

SEQ ID NO: 20 - DNA, 11 bp, Artificial Sequence
  Synthetic
ggggggggggg

SEQ ID NO: 21 - DNA, 12 bp, Artificial Sequence
  Synthetic
gggtggggtggg

SEQ ID NO: 22 - RNA, 12 nt, Artificial Sequence
  Synthetic
gggugggguggg

SEQ ID NO: 23 - DNA, 11 bp, Artificial Sequence
  Synthetic
ccccccccccc

SEQ ID NO: 24 - DNA, 12 bp, Artificial Sequence
  Synthetic
cccaccccaccc

SEQ ID NO: 25 - DNA, 7 bp, Artificial Sequence
  Synthetic
cgcgcgc

SEQ ID NO: 26 - DNA, 7 bp, Artificial Sequence
  Synthetic
gcgcgcg

SEQ ID NO: 27 - DNA, 6 bp, Artificial Sequence
  Synthetic
ttcttc

SEQ ID NO: 28 - RNA, 6 nt, Artificial Sequence
  Synthetic
uucuuc

SEQ ID NO: 29 - DNA, 6 bp, Artificial Sequence
  Synthetic
cttctt

SEQ ID NO: 30 - RNA, 6 nt, Artificial Sequence
  Synthetic
cuucuu

SEQ ID NO: 31 - DNA, 28 bp, Artificial Sequence
  Synthetic
ccggaattcggatccaaggtcgggcagg

SEQ ID NO: 32 - DNA, 82 bp, Artificial Sequence
  Synthetic
gcgaagcttaaaaatctagaaaaagggtgtggtgctagcaccacaccctttagatctcccgggtcgaccggtgtttcgttcc

SEQ ID NO: 33 - DNA, 20 bp, Artificial Sequence
  Synthetic
acgggaagaatctggtgcag

SEQ ID NO: 34 - DNA, 20 bp, Artificial Sequence
  Synthetic
ggcagcctctgtcatctcca

SEQ ID NO: 35 - DNA, 7 bp, Artificial Sequence
  Synthetic
aaaaaaa

SEQ ID NO: 36 - DNA, 8 bp, Artificial Sequence
  Synthetic
aaaaaaaa

SEQ ID NO: 37 - RNA, 7 nt, Artificial Sequence
  Synthetic
uuuuuuu

SEQ ID NO: 38 - RNA, 12 nt, Artificial Sequence
  Synthetic
agcuagcuagcu

SEQ ID NO: 39 - RNA, 19 nt, Artificial Sequence
  Synthetic
agcuagcuagcuaaaaaaa

SEQ ID NO: 40 - DNA, 9 bp, Artificial Sequence
  Synthetic
tttagctag

SEQ ID NO: 41 - RNA, 12 nt, Artificial Sequence
  Synthetic
aguacgcuagcu

SEQ ID NO: 42 - RNA, 19 nt, Artificial Sequence
  Synthetic
agcuagcguacuaaaaaaa

SEQ ID NO: 43 - RNA, 12 nt, Artificial Sequence
  Synthetic
agcuaccuagcu

SEQ ID NO: 44 - RNA, 17 nt, Artificial Sequence
  Synthetic
agcuagguagcuaaaaa

SEQ ID NO: 45 - RNA, 12 nt, Artificial Sequence
  Synthetic
agcuaccuagcu

SEQ ID NO: 46 - RNA, 12 nt, Artificial Sequence
  Synthetic
agcuagguagcu

SEQ ID NO: 47 - RNA, 20 nt, Artificial Sequence
  Synthetic
agcuagguagcuaaaaaaaa

SEQ ID NO: 48 - RNA, 15 nt, Artificial Sequence
  Synthetic
agcuagguagcuacc

SEQ ID NO: 49 - RNA, 12 nt, Artificial Sequence
  Synthetic
uuuauccaugga

SEQ ID NO: 50 - RNA, 8 nt, Artificial Sequence
  Synthetic
cccucuaa

SEQ ID NO: 51 - DNA, 6 bp, Artificial Sequence
  Synthetic
ggaggg

SEQ ID NO: 52 - RNA, 12 nt, Artificial Sequence
  Synthetic
uccauggauaaa

SEQ ID NO: 53 - RNA, 8 nt, Artificial Sequence
  Synthetic
cccucunn

SEQ ID NO: 54 - DNA, 21 bp, Artificial Sequence
  Synthetic
ctcgtggtggacttctctcaa

SEQ ID NO: 55 - DNA, 21 bp, Artificial Sequence
  Synthetic
ttgagagaagtccaccacgag

SEQ ID NO: 56 - DNA, 48 bp, Artificial Sequence
  Synthetic
ttgagagaagtccaccacgagtgtgtgctcgtggtggacttctctcaa

SEQ ID NO: 57 - RNA, 48 nt, Artificial Sequence
  Synthetic
uugagaggaguccaccacgagcacacacucgugguggacuucucucaa

SEQ ID NO: 58 - DNA, 7 bp, Artificial Sequence
  Synthetic
ccccccc

SEQ ID NO: 59 - DNA, 7 bp, Artificial Sequence
  Synthetic
ggggggg

SEQ ID NO: 60 - DNA, 6 bp, Artificial Sequence
  Synthetic
tgtgtg

SEQ ID NO: 61 - DNA, 62 bp, Artificial Sequence
  Synthetic
ttgagagaagtccaccacgaggggggggtgtgtgcccccccctcgtggtggacttctctcaa

SEQ ID NO: 62 - RNA, 62 nt, Artificial Sequence
  Synthetic
uugagagaaguccaccacgaggggggggcacacaccccccccucgugguggacuucucucaa

SEQ ID NO: 63 - DNA, 20 bp, Artificial Sequence
  Synthetic
gggggggtgtgtgccccccc

SEQ ID NO: 64 - RNA, 41 nt, Artificial Sequence
  Synthetic
uugagagaaguccaccacgaggggggggcacacaccccccc

SEQ ID NO: 65 - RNA, 50 nt, Artificial Sequence
  Synthetic
uugagagaaguccaccacgaggggggggcacacacccccccucguggugg

(19) United States
(12) Patent Application Publication
(10) Pub. No.: US 2009/0258930 A1
(43) Pub. Date: Oct. 15, 2009

(54) DOUBLE STRANDED RNA STRUCTURES AND CONSTRUCTS, AND METHODS FOR GENERATING AND USING THE SAME

(75) Inventors: Catherine J. PACHUK, Lansdale, PA (US); C. Satishchandran, Lansdale, PA (US); Maninder Chopra, Landenberg, PA (US); David Shuey, Berwyn, PA (US)

Correspondence Address: COOLEY GODWARD KRONISH LLP;ATTN: Patent Group, Suite 1100, 777 - 6th Street, NW, WASHINGTON, DC, 20001, US

(73) Assignee: Nucleonics, Inc.

(21) Appl. No.: 12/246,258

(22) Filed: Oct. 6, 2008

Related U.S. Application Data
(63) Continuation of application No. 10/522,962, filed on Jan. 31, 2005, now abandoned.
(60) Provisional application No. 60/399,998, filed on Jul. 31, 2002.

(51) Int. Cl. A61K 31/7088; C07H 21/00; C12P 19/34; A61P 35/00

(52) U.S. Cl. 514/44 R; 536/23.1; 435/91.3

(57) ABSTRACT

The present invention relates to novel double stranded RNA (dsRNA) structures and dsRNA expression constructs, methods for generating them, and methods of utilizing them for silencing genes. Desirably, these methods specifically inhibit the expression of one or more target genes in a cell or animal (e.g., a mammal such as a human) without inducing toxicity. These methods can be used to prevent or treat a disease or infection by silencing a gene associated with the disease or infection. The invention also provides methods for identifying nucleic acid sequences that modulate a detectable phenotype, such as the function of a cell, the expression of a gene, or the biological activity of a target polypeptide.

### BACKGROUND OF THE INVENTION

In general, the invention relates to novel double stranded RNA (dsRNA) structures and dsRNA expression constructs, methods for generating them, and methods of utilizing them for silencing genes. Desirably, these methods specifically inhibit the expression of one or more target genes in a eukaryotic cell, plant, or animal (e.g., a mammal, such as a human) without inducing toxicity.

Double stranded RNA (dsRNA) has been shown to induce gene silencing in a number of different organisms. Gene silencing can occur through various mechanisms, one of which is post-transcriptional gene silencing (PTGS). In post-transcriptional gene silencing, transcription of the target locus is not affected, but the RNA half-life is decreased. Exogenous dsRNA has been shown to act as a potent inducer of PTGS in plants and animals, including nematodes, trypanosomes, and insects. Transcriptional gene silencing (TGS) is another mechanism by which gene expression can be regulated. In TGS, transcription of a gene is inhibited. The potential to harness dsRNA mediated gene silencing for research, therapeutic, and prophylactic indications is enormous. The exquisite sequence specificity of target mRNA degradation and the systemic properties associated with PTGS make this phenomenon ideal for functional genomics and drug development.

Some current methods for using dsRNA in vertebrate cells to silence genes result in undesirable non-specific cytotoxicity or cell death due to dsRNA-mediated stress responses, including the interferon response. A potential quagmire exists for the use of RNAi in vertebrate systems, including humans, because of the ability of dsRNA to trigger various toxicities in vertebrates, e.g., the type I interferon response as well as other RNA stress response pathways. Induction of a dsRNA-mediated stress response is rapid, and may result in cellular apoptosis or anti-proliferative effects. In addition to the potential for dsRNA to trigger toxicity in vertebrate cells, dsRNA gene silencing methods may result in non-specific or inefficient silencing.

Another hurdle facing the practical implementation of dsRNA is the inefficient production and delivery of dsRNA structures, e.g., problems of inefficient production of dsRNAs from dsRNA expression constructs. One such problem involves the inefficient production of “hairpin” dsRNAs (which have sense and antisense sequences within a single strand), including problems of expression from dsRNA expression constructs encoding such “hairpin” dsRNAs. Thus, improved methods are needed for specifically and efficiently silencing target genes without inducing toxicity or cell death. Furthermore, there is a need for improved dsRNA “hairpin” constructs, expression constructs for producing such dsRNA hairpin structures, and methods for using the same. Desirably, these methods may be used to inhibit gene expression in in vitro samples, cell culture, and intact animals (e.g., vertebrates, such as mammals).

### SUMMARY OF THE INVENTION

### Nucleic Acids

The invention features nucleic acids and populations of nucleic acids that have particular secondary structures, such as those illustrated in FIGS. 1A-1D and 2A-2C. These populations of nucleic acids may be used in a variety of screening methods to isolate nucleic acids that have the ability to inhibit the expression of a target nucleic acid. Additionally, the nucleic acids can be used in any of the methods of the invention to silence a target gene in a eukaryotic cell, plant, or animal.

Forced Hairpins

In one such aspect, the invention features a nucleic acid (e.g., a DNA molecule or vector) or a population of nucleic acids encoding an RNA (e.g., a partial or full hairpin) that has, in 5′ to 3′ order, a first region of interest, a first base-paired region, a loop region, and a second base-paired region. The first and second base-paired regions are base-paired to each other. Desirably, the nucleic acid further includes a second region of interest downstream of the second base-paired region. If the second region of interest is present, the first and second regions of interest are base-paired to each other. Desirably, at least 50, 60, 70, 80, 90, 95, or 100% of the nucleotides in first and second regions of interest participate in Watson-Crick base-pairing with each other. These two regions between may be the same length or may differ in length by one or more nucleotides. For example, one region of interest may have additional nucleotides at one end of the region that are not base-paired to nucleotides in any portion of the other region of interest. In a related aspect, the invention features an RNA molecule or a population of RNA molecules encoded by these nucleic acids. Exemplary RNA molecules are illustrated in FIGS. 1A-1D, in which the first and second base-paired regions are denoted “A” and “B,” and the first and second regions of interest are denoted “sense” and “antisense.”

In desirable embodiments of the above aspects, one region of interest has substantial identity to a target gene, and the other region of interest has substantial complementarity to the target gene. Desirably, the encoded RNA inhibits expression of the target gene in a cell or animal. In desirable embodiments, the number of RNA molecules that adopt a hairpin structure in vivo or in vitro in which the regions of interest are base-paired to each other is at least 25, 50, 75, 100, 200, 500, or even 1000% greater than the number of control RNA molecules (e.g., molecules lacking the first and second base-paired regions) that adopt such a hairpin structure under the same conditions.

Partial Hairpins

In another aspect, the invention features a nucleic acid (e.g., a DNA molecule or vector) or a population of nucleic acids encoding a partial RNA hairpin that has a single stranded overhang. The encoded RNA molecule has, in 5′ to 3′ order, a first region of interest, a loop region, and a second region of interest. The regions of interest differ in length, and one region of interest has additional nucleotides at one end of the region that are not base-paired to nucleotides in the other region of interest. One region of interest has substantial identity to a target gene, and the other region of interest has substantial complementarity to the target gene. In a related aspect, the invention features an RNA molecule or a population of RNA molecules encoded by these nucleic acids. Exemplary RNA molecules are illustrated in FIG. 2A in which the first and second regions of interest are denoted “sense” and “antisense.” Desirably, the encoded RNA inhibits expression of the target gene in a cell or animal.

Methods for Producing Hairpins

The invention also provides methods for generating hairpins. These methods involve producing a partial hairpin that has a single stranded overhang and extending the partial hairpin so that the single stranded overhang decreases in size. In desirable embodiments, the 3′ end of the partial hairpin is extended such that a full hairpin without an overhang is generated.

In one such aspect, the invention features a method for generating an RNA hairpin. This method involves extending the 3′ end of a partial hairpin that has a 3′ end that is base-paired with another region in the partial hairpin and that has a 5′ overhang. The partial hairpin is extended in vitro or in vivo (e.g., in a cell or animal) with an RNA dependent-RNA polymerase. Desirably, the extension of the partial hairpin produces a full hairpin.

In some embodiments, the partial hairpin has, in 5′ to 3′ order, a first region of interest, a first base-paired region, a loop region, and a second base-paired region. In this case, the first and second base-paired regions are base-paired to each other. The partial hairpin may also have a second region of interest that (i) is downstream of the second base-paired region, (ii) is shorter in length than the first region of interest, and (iii) participates in base-pairing with the first region of interest. Desirably, the second region of interest is extended such that it is the same length as the first region of interest. Any of the RNA molecules of the above aspects in which the second region of interest is absent or is shorter that the first region of interest can be used in this method, provided that nucleotides near or at the 3′ terminus of the RNA molecule participate in intramolecular base-pairing. Desirably, at least 5, 6, 8, 10, 15, or more of the very last 3′ terminal nucleotides participate in base-pairing. Desirably, the synthesized hairpin has one region of interest with substantial identity to a target gene, and another region of interest with substantial complementarity to the target gene. Desirably, the synthesized hairpin inhibits expression of the target gene in a cell or animal. In some embodiments, the cell or animal in which gene silencing occurs is administered a partial hairpin or a nucleic acid encoding the partial hairpin, and the partial hairpin is extended in vivo. In this case, if the cell or animal does not already express an RNA dependent-RNA polymerase, an RNA dependent-RNA polymerase or a nucleic acid encoding an RNA dependent-RNA polymerase is also administered.

In some embodiments, the initial, partial hairpin is generated by transcription of a DNA molecule with a transcription termination sequence that results in the production of a partial hairpin that terminates in a sequence (e.g., a sequence of at least 5, 10, 15, 20, 30, 40, 50, 100, or more nucleotides) that is substantially complementary to a region within the partial RNA such that at least 5, 6, 8, 10, 15 or more of the 3′ terminal nucleotides of the partial hairpin participate in intramolecular base-pairing. In particular embodiments, the partial hairpin is transcribed by RNA polIII in the nucleus and terminates in at least 4, 5, 6, 8, or more T nucleotides of the DNA template. In this case, at least 4, 5, 6, 8, 10, or more of these nucleotides base-pair with “A” nucleotides within the partial hairpin.

In other embodiments, the initial, partial hairpin is generated by enzymatic cleavage of a longer RNA molecule (e.g., a longer partial hairpin with nucleotides at the 3′ terminus to be removed). In some embodiments, the enzyme (e.g., a restriction enzyme or a ribozyme) cleaves the longer RNA molecule at a specific site (e.g., a restriction enzyme or ribozyme cleavage site) to remove nucleotides from the 3′ end of the molecule, thereby generating a partial hairpin with a 3′ terminus that participates in intramolecular base-pairing. In particular embodiments, a ribozyme is located in a loop of a longer RNA molecule, and nucleotides at the 3′ end of the RNA molecule are removed by ribozyme-mediated cleavage in cis to generate the partial hairpin. In other embodiments, the ribozyme is located at or near the 3′ terminus of a longer RNA molecule and cleaves the RNA molecule in cis at a position upstream of the ribozyme to generate a partial hairpin without the ribozyme. In other embodiments, the ribozyme is a separate molecule that cleaves the longer RNA molecule in trans to generate the partial hairpin. Exemplary ribozymes include hairpin, hammerhead, self-splicing (e.g., tetrahymena or phage T4 td intron), and HDV- or RNase P-mediated ribozymes.

In still other embodiments, the initial, partial hairpin is produced by hybridizing a DNA molecule to a longer RNA molecule, and cleaving the DNA/RNA hybrid with an enzyme such as RNAse H. One or more of the cleavage products are the desired partial hairpins.

### Desirable Embodiments of Nucleic Acids of Above Aspects

In various embodiments of any of the above aspects, the first and second base-paired regions are the same length or differ in length by one or more nucleotides. In some embodiments, the first and/or second base-paired regions are between 5-15 nucleotides, 16-25 nucleotides, 26-50 nucleotides, 51-75 nucleotides, 76-100 nucleotides, 101-150 nucleotides, 151-200 nucleotides, 201-300 nucleotides, 301-400 nucleotides, or 401-1000 nucleotides in length, inclusive. Desirably, at least 50, 60, 70, 80, 90, 95, or 100% of the nucleotides in first and second base-paired regions participate in Watson-Crick base-pairing with each other. In other desirable embodiments, less than 30, 20, 10, or 5% of the nucleotides in the first base-paired region base-pair with other nucleotides in the first base-paired region, and less than 30, 20, 10, or 5% of the nucleotides in the second base-paired region base-pair with other nucleotides in the second base-paired region. In some embodiments, the first and/or second regions of interest are between 5-15 nucleotides, 15-25 nucleotides, 19-26 nucleotides, 25-50 nucleotides, 50-75 nucleotides, 75-100 nucleotides, 100-150 nucleotides, 150-200 nucleotides, 200-300 nucleotides, 300-400 nucleotides, or 400-1000 nucleotides in length, inclusive. In other embodiments, the regions of interest are at least 1000, 2000, 3000, 4000, 5000, 8000, 10000, or more nucleic acids in length. If desired, some or all of the nucleotides in the loop of may be randomized. In various embodiments, the loop contains at least 5, 7, 10, 20, 30, 60, 100, or more nucleotides. In some embodiments, the loop has less than 10000, 8000, 7000, 5000, 1000, 500, or 200 nucleotides. Desirably, less than 30, 20, 10, or 5 of the nucleotides in the loop base-pair with other nucleotides in the loop or with nucleotides in the first or second base-paired region. The partial or full hairpin may also have other secondary structures, such as other loops, base-paired helices, or additional sequences at either end of the molecule.

In other preferred embodiments, the population of nucleic acids contains more than one DNA molecule or more than one RNA molecule. The nucleic acids may have naturally-occurring or non-naturally-occurring polynucleotide sequences. In some embodiments, regions of the nucleic acids; such as all or part of a loop, the first and second regions of interest, or the first and second base-paired regions; contain sequences that differ between some or all of the members of the population. In other embodiments, the sequence of the loop region and/or the first and second regions of interest is the same in all of the members of the population. The lengths of the loop or any of the other regions may be the same or may differ between members of the population. The populations of nucleic acids may contain any number of unique molecules. For example, the population may contain as few as 10, 10², 10⁹, or 10¹¹ unique molecules or as many as 10¹³, 10¹⁴, 10¹⁵ or more unique molecules. In desirable embodiments, at least one of the polynucleotide sequences is a non-naturally-occurring sequence. Desirably, at least 10, 20, 40, 60, 80, 90, 95, 98, or 100% of the unique polynucleotide sequences are non-naturally-occurring sequences. The nucleic acids may either all have the same length or some of the molecules may differ in length. Preferably, the nucleic acids contain at least 50, 100, 200, 500, 1000, or more bases.

Pharmaceutical Compositions

The invention also features pharmaceutical compositions that include one or more dsRNA molecules or nucleic acids encoding dsRNA molecules (e.g., partial or full hairpins) in an acceptable vehicle. In one such aspect, the invention features a pharmaceutical composition that includes one or more nucleic acids of any of the aspects of the invention in an acceptable vehicle.

In another aspect, the invention provides a pharmaceutical composition which includes at least one short dsRNA (e.g., 1, 2, 3, 5, 8, 10, 20, 30, or more different short dsRNA species) and at least one long dsRNA (e.g., 1, 2, 3, 5, 8, 10, 20, 30, or more different long dsRNA species) in an acceptable vehicle (e.g., a pharmaceutically acceptable carrier).

In various embodiments, the pharmaceutical composition includes about 1 ng to about 20 mg of nucleic acid, e.g., RNA, DNA, plasmids, viral vectors, recombinant viruses, or mixtures thereof, which provide the desired amounts of the respective dsRNA molecules (dsRNA homologous to a target nucleic acid and/or dsRNA to inhibit toxicity). In some embodiments, the composition contains about 10 ng to about 10 mg of nucleic acid, about 0.1 mg to about 500 mg, about 1 mg to about 350 mg, about 25 mg to about 250 mg, or about 100 mg of nucleic acid. If desired, the dosage regimen of the short dsRNA may be adjusted to achieve the optimal inhibition of PKR and/or other dsRNA-mediated stress responses, and the dosage regimen of the other dsRNA (e.g, long dsRNA) may be adjusted to optimize the desired sequence-specific silencing. Accordingly, a composition of the invention may contain different amounts of the two dsRNA molecules. Those of skill in the art of clinical pharmacology can readily arrive at such dosing schedules using routine experimentation.

Suitable carriers include, but are not limited to, saline, buffered saline, dextrose, water, glycerol, ethanol, and combinations thereof. The composition can be adapted for the mode of administration and can be in the form of, for example, a pill, tablet, capsule, spray, powder, or liquid. In some embodiments, the pharmaceutical composition contains one or more pharmaceutically acceptable additives suitable for the selected route and mode of administration. These compositions may be administered by, without limitation, any parenteral route including intravenous, intra-arterial, intramuscular, subcutaneous, intradermal, intraperitoneal, intrathecal, as well as topically, orally, and by mucosal routes of delivery such as intranasal, inhalation, rectal, vaginal, buccal, and sublingual. In some embodiments, the pharmaceutical compositions of the invention are prepared for administration to vertebrate (e.g., mammalian) subjects in the form of liquids, including sterile, non-pyrogenic liquids for injection, emulsions, powders, aerosols, tablets, capsules, enteric coated tablets, or suppositories.

Kits for Synthesis or Administration of dsRNA Molecules

In a related aspect, the invention provides a kit for generation of a hairpin. The kit includes (i) a partial hairpin or a nucleic acid encoding a partial hairpin and (i) an RNA dependent-RNA polymerase or a nucleic acid encoding an RNA dependent-RNA polymerase. In desirable embodiments, the kit also includes a means for removing nucleotides from the 3′ terminus of the partial hairpin to generate a partial hairpin in which some or all of the nucleotides in the 3′ terminus participate in intramolecular base-pairing (e.g., a restriction enzyme, a ribozyme, or RNAse H and a DNA molecule that hybridizes to the partial hairpin).

In a related aspect, the invention provides a kit which includes at least one short dsRNA (e.g., 1, 2, 3, 5, 8, 10, 20, 30 or more different short dsRNA species) in an acceptable vehicle and at least one long dsRNA (e.g., 1, 2, 3, 5, 8, 10, 20, 30, or more different long dsRNA species) in an acceptable vehicle. The kit allows the short dsRNA to be administered before, simultaneously with, or after the long dsRNA. In some embodiments, the short dsRNA is administered using a different route, delivery system, mode, site, or rate of administration that used for the long dsRNA. In some embodiments, the short dsRNA and/or the long dsRNA is a forced or partial hairpin or a nucleic acid encoding a forced or partial hairpin as described herein.

Cells with Nucleic Acids of the Invention

The invention also features cells with one or more of the nucleic acids of the invention. In one such aspect, the invention features a cell or a population of cells that expresses a dsRNA that modulates a detectable phenotype, including, without limitation, a dsRNA that: (i) modulates a function of the cell, (ii) modulates the expression of a target nucleic acid (e.g., an endogenous or pathogen gene) in the cell, and/or (iii) modulates the biological activity of a target protein (e.g., an endogenous or pathogen protein) in the cell. In some embodiments, this dsRNA is generated in vivo by the extension of a partial hairpin using a method of the invention; for example, the cell may express an endogenous or exogenous RNA dependent-RNA polymerase and a partial hairpin that is extended in vivo. In other embodiments, this dsRNA has, in 5′ to 3′ order, a first region of interest (i.e., a region with substantial identity or complementarity to a target gene), a first base-paired region, a loop region, a second base-paired region, and a second region of interest. The first and second base-paired regions are base-paired to each other, and the first and second regions of interest are base-paired to each other. In other embodiments, the dsRNA is encoded by a vector that has an origin of replication that permits replication of the vector in the cell. Desirably, the vector is maintained in the progeny of the cell after 1, 5, 10, 15, 30, 50, 100, or more cell divisions.

In desirable embodiments, the cell or population of cells also has one or more short dsRNA molecules (e.g., 1, 2, 3, 5, 8, 10, 20, 30, or more different short dsRNA species) that desirably inhibit an interferon response or a dsRNA stress response by the former dsRNA. In some embodiments, the cell contains only one molecular species of long dsRNA or only one copy of a dsRNA expression vector encoding a long dsRNA (e.g., a stably integrated vector). Desirably, the cell or population of cells is produced using one or more methods of the invention. In other embodiments, the dsRNA is expressed under conditions that inhibit or prevent an interferon response or a dsRNA stress response.

Methods for Inhibiting Gene Expression in Cells or Animals

The invention also features novel methods for silencing genes that produce few, if any, toxic side-effects. In particular, these methods involve administering to a cell or animal an agent that provides one or more double stranded RNA (dsRNA) molecules that have substantial sequence identity to a region of a target nucleic acid and that specifically inhibit the expression of the target nucleic acid. If desired, an agent that provides one or more short dsRNA molecules, which differ from the dsRNA having substantial identity to the target nucleic acid, is also administered to inhibit possible toxic effects or non-specific gene silencing that may otherwise be induced by the former dsRNA. In some embodiments, the agent is a nucleic acid or pharmaceutical composition of any of the above aspects.

Accordingly, in one such aspect, the invention features a method for inhibiting the expression of a target nucleic acid in a cell (e.g., a eukaryotic cell, a plant cell, an animal cell, an invertebrate cell, a vertebrate cell, such as a mammalian or human cell, or a pathogen cell). This method involves introducing into the cell a first agent that provides to the cell a first dsRNA that has substantial sequence identity to a region of the target nucleic acid and specifically inhibits the expression of the target nucleic acid. Exemplary pathogens include bacteria and yeast. In some embodiments, the first dsRNA inhibits the expression of an endogenous nucleic acid in a vertebrate cell or a pathogen cell (e.g., a bacterial or yeast cell) or inhibits the expression of a pathogen nucleic acid in a cell infected with the pathogen.

In some embodiments of the above aspect, a second agent that provides to the cell a short, second dsRNA is also introduced into the cell. The short, second dsRNA differs from the first dsRNA and inhibits the interferon response or dsRNA-mediated toxicity. In some embodiments, the short, second dsRNA binds PKR and inhibits the dimerization and/or activation of PKR.

In another aspect, the invention provides a method for inhibiting the expression of a target nucleic acid in an animal (e.g., an invertebrate or a vertebrate such as a mammal or human). This method involves introducing into the animal a first agent that provides to the animal a first dsRNA. The first dsRNA has substantial sequence identity to a region of the target nucleic acid and specifically inhibits the expression of the target nucleic acid. In some embodiments, the first dsRNA inhibits the expression of an endogenous nucleic acid in an animal or inhibits the expression of a pathogen nucleic acid in an animal infected with a pathogen (e.g., a bacteria, yeast, or virus).

In some embodiments of the above aspect, a second agent that provides to the animal a short, second dsRNA is administered to the animal. The short, second dsRNA differs from the first dsRNA and inhibits the interferon response or dsRNA-mediated toxicity. In some embodiments, the short, second dsRNA binds PKR and inhibits the dimerization and/or activation of PKR. In some embodiments of the above aspects, the short dsRNA and/or the long dsRNA is a forced or partial hairpin or a nucleic acid encoding a forced or partial hairpin as described herein.

Methods for Treating or Preventing Disease by Inhibiting Gene Expression Animals

In yet another aspect, the invention provides a method for treating, stabilizing, or preventing a disease or disorder in an animal (e.g., an invertebrate or a vertebrate such as a mammal or human). This method involves introducing into the animal a first agent that provides to the animal a first dsRNA. The first dsRNA has substantial sequence identity to a region of a target nucleic acid associated with the disease or disorder and specifically inhibits the expression of the target nucleic acid. In some embodiments, the target gene is a gene associated with cancer, such as an oncogene, or a gene encoding a protein associated with a disease, such as a mutant protein, a dominant negative protein, or an overexpressed protein.

In various embodiments of the above aspect, a second agent that provides to the animal a short, second dsRNA is also administered to the animal. The short, second dsRNA differs from the first dsRNA and inhibits the interferon response or dsRNA-mediated toxicity. In some embodiments, the short, second dsRNA binds PKR and inhibits the dimerization and/or activation of PKR. In some embodiments of the above aspects, the short dsRNA and/or the long dsRNA is a forced or partial hairpin or a nucleic acid encoding a forced or partial hairpin as described herein.

Exemplary cancers that can be treated, stabilized, or prevented using the above methods include prostate cancers, breast cancers, ovarian cancers, pancreatic cancers, gastric cancers, bladder cancers, salivary gland carcinomas, gastrointestinal cancers, lung cancers, colon cancers, melanomas, brain tumors, leukemias, lymphomas, and carcinomas. Benign tumors may also be treated or prevented using the methods of the present invention. Other cancers and cancer related genes that may be targeted are disclosed in, for example, WO 00/63364, WO 00/44914, and WO 99/32619.

Exemplary endogenous proteins that may be associated with disease include ANA (anti-nuclear antibody) found in SLE (systemic lupus erythematosis), abnormal immunoglobulins including IgG and IgA, Bence Jones protein associated with various multiple myelomas, and abnormal amyloid proteins in various amyloidoses including hereditary amyloidosis and Alzheimer's disease. In Huntington's Disease, a genetic abnormality in the HD (huntingtin) gene results in an expanded tract of repeated glutamine residues. In addition to this mutant gene, HD patients have a copy of chromosome 4 which has a normal sized CAG repeat. Thus, methods of the invention can be used to silence the abnormal gene, but not the normal gene. In various embodiments, a nucleic acid encoding a disease-causing protein is silenced using long dsRNA, and short dsRNA is used to block the dsRNA stress response that might otherwise be associated with administration of the long dsRNA.

Methods for Treating or Preventing Infection by Inhibiting Gene Expression Animals

In still another aspect, the invention features a method for treating, stabilizing, or preventing an infection in an animal (e.g., an invertebrate or a vertebrate such as a mammal or human). This method involves introducing into the animal a first agent that provides to the animal a first dsRNA. The first dsRNA has substantial sequence identity to a region of a target nucleic acid in an infectious pathogen (e.g., a virus, bacteria, or yeast) or in a cell infected with a pathogen and specifically inhibits the expression of the target nucleic acid. In various embodiments, the pathogen is an intracellular or extracellular pathogen. In some embodiments, the target nucleic acid is a gene of the pathogen that is necessary for replication and/or pathogenesis, or a gene encoding a cellular receptor necessary for a cell to be infected with the pathogen.

In some embodiments of the above aspect, a second agent that provides to the animal a short, second dsRNA is also administered to the animal. The short, second dsRNA differs from the first dsRNA and inhibits the interferon response or dsRNA-mediated toxicity. In some embodiments, the short, second dsRNA binds PKR and inhibits the dimerization and/or activation of PKR. In some embodiments of the above aspects, the short dsRNA and/or the long dsRNA is a forced or partial hairpin or a nucleic acid encoding a forced or partial hairpin as described herein.

In further embodiments of any of the above aspects, the methods of administering a dsRNA or a nucleic acid encoding a dsRNA includes contacting an in-dwelling device with the agent prior to, concurrent with, or following the administration of the in-dwelling device to a patient. In-dwelling devices include, but are not limited to, surgical implants, prosthetic devices, and catheters, i.e., devices that are introduced to the body of an individual and remain in position for an extended time. Such devices include, for example, artificial joints, heart valves, pacemakers, vascular grafts, vascular catheters, cerebrospinal fluid shunts, urinary catheters, and continuous ambulatory peritoneal dialysis (CAPD) catheters. Desirably, the dsRNA prevents the growth of bacteria on the device. In some embodiments, the first dsRNA inhibits the expression of a bacterial nucleic acid in a bacterial cell, a cell infected with a bacterium, or an animal infected with a bacterium.

In other desirable embodiments, the bacterial infection is due to one or more of the following bacteria: Chlamydophila pneumoniae, C. psittaci, C. abortus, Chlamydia trachomatis, Simkania negevensis, Parachlamydia acanthamoebae, Pseudomonas aeruginosa, P. alcaligenes, P. chlororaphis, P. fluorescens, P. luteola, P. mendocina, P. monteilii, P. oryzihabitans, P. pertocinogena, P. pseudalcaligenes, P. putida, P. stutzeri, Burkholderia cepacia, Aeromonas hydrophilia, Escherichia coli, Citrobacter freundii, Salmonella typhimurium, S. typhi, S. paratyphi, S. enteritidis, Shigella dysenteriae, S. flexneri, S. sonnei, Enterobacter cloacae, E. aerogenes, Klebsiella pneumoniae, K. oxytoca, Serratia marcescens, Francisella tularensis, Morganella morganii, Proteus mirabilis, Proteus vulgaris, Providencia alcalifaciens, P. rettgeri, P. stuartii, Acinetobacter calcoaceticus, A. haemolyticus, Yersinia enterocolitica, Y. pestis, Y. pseudotuberculosis, Y. intermedia, Bordetella pertussis, B. parapertussis, B. bronchiseptica, Haemophilus influenzae, H. parainfluenzae, H. haemolyticus, H. parahaemolyticus, H. ducreyi, Pasteurella multocida, P. haemolytica, Branhamella catarrhalis, Helicobacter pylori, Campylobacter fetus, C. jejuni, C. coli, Borrelia burgdorferi, V. cholerae, V. parahaemolyticus, Legionella pneumophila, Listeria monocytogenes, Neisseria gonorrhea, N. meningitidis, Kingella dentrificans, K. kingae, K oralis, Moraxella catarrhalis, M. atlantae, M. lacunata, M. nonliquefaciens, M. osloensis, M. phenylpyruvica, Gardnerella vaginalis, Bacteroides fragilis, Bacteroides distasonis, Bacteroides 3452A homology group, Bacteroides vulgatus, B. ovalus, B. thetaiotaomicron, B. uniformis, B. eggerthii, B. splanchnicus, Clostridium difficile, Mycobacterium tuberculosis, M. avium, M intracellulare, M. leprae, C. diphtheriae, C. ulcerans, C. accolens, C afermentans, C. amycolatum, C. argentorense, C. auris, C. bovis, C confusum, C. coyleae, C. durum, C. falsenii, C. glucuronolyticum, C. imitans, C. jeikeium, C. kutscheri, C. kroppenstedtii, C. lipophilum, C. macginleyi, C. matruchoti, C. mucifaciens, C. pilosum, C. propinquum, C. renale, C. riegelii, C. sanguinis, C. singulare, C. striatum, C. sundsvallense, C. thomssenii, C. urealyticum, C. xerosis, Streptococcus pneumoniae, S. agalactiae, S. pyogenes, Enterococcus avium, E. casseliflavus, E. cecorum, E. dispar, E. durans, E. faecalis, E. faecium, E. flavescens, E. gallinarum, E. hirae, E. malodoratus, E. mundtii, E. pseudoavium, E. raffinosus, E. solitarius, Staphylococcus aureus, S. epidermidis, S. saprophyticus, S. intermedius, S. hyicus, S. haemolyticus, S. hominis, and/or S. saccharolyticus. Preferably, a dsRNA is administered in an amount sufficient to prevent, stabilize, or inhibit the growth of a pathogen or to kill the pathogen. In some embodiments, the first dsRNA inhibits the expression of a yeast nucleic acid in a yeast cell, a cell infected with yeast, or an animal infected with yeast.

In desirable embodiments, the viral infection relevant to the methods of the invention is an infection by one or more of the following viruses: Hepatitis B, Hepatitis C, picomarirus, polio, HIV, coxsacchie, herpes simplex virus Type I and 2, St. Louis encephalitis, Epstein-Barr, myxoviruses, JC, coxsakieviruses B, togaviruses, measles, paramyxoviruses, echoviruses, bunyaviruses, cytomegaloviruses, varicella-zoster, mumps, equine encephalitis, lymphocytic choriomeningitis, rhabodoviruses including rabies, simian virus 40, human polyoma virus, parvoviruses, papilloma viruses, primate adenoviruses, coronaviruses, retroviruses, Dengue, yellow fever, Japanese encephalitis virus, and/or BK. In some embodiments, the first dsRNA inhibits the expression of a viral nucleic acid in a cell or animal infected with a virus.

Particularly suitable for the therapeutic and prophylactic methods of the invention are DNA viruses or viruses that have an intermediary DNA stages. Among such viruses are included, without limitation, viruses of the species Retrovirus, Herpesvirus, Hepadenovirus, Poxvirus, Parvovirus, Papillomavirus, and Papovavirus. Specifically some of the more desirable viruses to treat with this method include, without limitation, HIV, BBV, HSV, CMV, HPV, HTLV and EBV. The agent used in this method provides to the cell of the mammal an at least partially double stranded RNA molecule as described herein, which includes a double-stranded sequence substantially homologous to a target polynucleotide which is a virus polynucleotide sequence necessary for replication and/or pathogenesis of the virus in an infected mammalian cell. Among such target polynucleotide sequences are protein encoding sequences for proteins necessary for the propagation of the virus, e.g., the HIV gag, env, and pol genes, the HPV6 LI and E2 genes, the HPV I I LI and E2 genes, the HPV 16 E6 and E7 genes, the BPV 18 E6 and E7 genes, the HBV surface antigens, the HBV core antigen, HBV reverse transcriptase, the HSV gD gene, the HSVvp 16 gene, the HSV gC, gH, gL and gB genes, the HSV ICPO, ICP4 and ICP6 genes, Varicella zoster gB, gC and gH genes, and the BCR-abl chromosomal sequences, and non-coding viral polynucleotide sequences which provide regulatory functions necessary for transfer of the infection from cell to cell, e.g., the HIV LTR, and other viral promoter sequences, such as HSV vp 16 promoter, HSV-ICPO promoter, HSV-ICP4, ICP6 and gD promoters, the HBV surface antigen promoter, the HBV pre-genomic promoter, among others.

Thus, this method can be used to treat mammalian subjects already infected with a virus, such as HIV, in order to shut down or inhibit a viral gene function essential to virus replication and/or pathogenesis, such as HIV gag. Alternatively, this method can be employed to inhibit the functions of viruses which exist in mammals as latent viruses, e.g., Varicella zoster virus, and are the causative agents of the disease known as shingles. Similarly, diseases such as atherosclerosis, ulcers, chronic fatigue syndrome, and autoimmune disorders, recurrences of HSV-I and HSV-2, HPV persistent infection, e.g., genital warts, and chronic BBV infection among others, which have been shown to be caused, at least in part, by viruses, bacteria, or another pathogen, can be treated according to this method by targeting certain viral polynucleotide sequences essential to viral replication and/or pathogenesis in the mammalian subject.

Still another analogous embodiment of the above “anti-viral” methods of the invention includes a method for treatment or prophylaxis of a virally induced cancer in a mammal. Such cancers include HPV E6/E7 virus-induced cervical carcinoma, HTLV-induced cancer, and EBV induced cancers, such as Burkitts lymphoma, among others. This method is accomplished by administering to the mammal a composition, as described herein, in which the target polynucleotide is a sequence encoding a tumor antigen or functional fragment thereof, or a non-expressed regulatory sequence, which antigen or sequence function is required for the maintenance of the tumor in the mammal. Among such sequences are included, without limitation, HPV16 E6 and E7 sequences and HPV 18 E6 and E7 sequences. Others may readily be selected by one of skill in the art. The composition is administered in an amount effective to reduce or inhibit the function of the antigen in the mammal, and preferably employs the composition components, dosages, and routes of administration as described herein.

In another aspect, the invention features a method for reducing or preventing an immune response to a transplant cell, tissue, or organ. The method involves administering to the transplant cell, tissue, or organ a first agent that provides a first dsRNA. The first dsRNA attenuates the expression of a target nucleic acid in the transplant cell, tissue, or organ that can elicit an immune response in a recipient. In some embodiments, an agent that provides a dsRNA molecule is also administered to the recipient to inhibit the expression of an endogenous nucleic acid that would otherwise participate in an adverse immune response to the transplant.

In various embodiments of the above aspect, a second agent that provides a short, second dsRNA is also administered to the transplant cell, tissue, or organ. See the teaching of U.S. Ser. No. 60/375,636, filed Apr. 26, 2002 and U.S. Ser. No. 10/425,006 filed Apr. 28, 2003, “Methods of Silencing Genes Without Inducing Toxicity”, C. Pachuk, incorporated herein by reference. The short, second dsRNA differs from the first dsRNA and inhibits the interferon response or dsRNA-mediated toxicity. In some embodiments, the short, second dsRNA binds PKR and inhibits the dimerization and/or activation of PKR

In desirable embodiments of any of the above aspects, the first dsRNA inhibits expression of the target nucleic acid by at least 20, 40, 60, 80, 90, 95, or 100%. In some embodiments, multiple first dsRNA molecules that are substantially identical to different nucleic acids are administered to the cell or animal to inhibit the expression of multiple target nucleic acids. In other embodiments, a multiple epitope first dsRNA that has segments with substantial identity to different target genes is administered to silence multiple target genes. For example, multiple oncogenes or multiple pathogen genes may be simultaneously silenced.

In various embodiments of any of the above aspects, the first agent and/or the second agent is a DNA molecule or DNA vector encoding a dsRNA. In other embodiments, the first agent and/or the second agent is a dsRNA, a single stranded RNA molecule that assumes a double stranded conformation inside the cell or animal (e.g., a partial or full hairpin), or a combination of two single stranded RNA molecules that are administered simultaneously or sequentially and that assume a double stranded conformation inside the cell or animal. The first agent may be administered before, during, or after the administration of the second agent. In some embodiments, the first and second agents are the same nucleic acid or the same vector that encodes both dsRNA molecules. In various embodiments, the first agent provides a short dsRNA or a long dsRNA to the cell or animal. In some embodiments of the above aspects, the short dsRNA and/or the long dsRNA is a forced or partial hairpin or a nucleic acid encoding a forced or partial hairpin as described herein.

In some embodiments, a cytokine is also administered to the cell or animal. Exemplary cytokines are disclosed in WO 00/63364, filed Apr. 19, 2000. In some embodiments, the expression of the target nucleic acid is increased to promote the amplification of the dsRNA, resulting in more dsRNA to silence the target gene. For example, a vector containing the target nucleic acid can be administered to the cell or animal before, during, or after the administration of the first and/or second agent.

Methods for Identifying Nucleic Acids of Interest by Transfecting Cells with a dsRNA Expression Library

The invention also features high throughput methods of using dsRNA-mediated gene silencing to identify a nucleic acid associated with a detectable phenotype in a cell, e.g., a nucleic acid that modulates the function of a cell, gene expression of a target nucleic acid, or the biological activity of a target polypeptide herein. The method involves the use of specially constructed cDNA libraries derived from a cell (for example, a primary cell or a cell line that has an observable phenotype or biological activity e.g., an activity mediated by a target polypeptide or altered gene expression) that are transfected into cells to inhibit gene expression. The inhibition of gene expression by the present methods alters a detectable phenotype, e.g., the function of a cell, gene expression of a target nucleic acid, or the biological activity of a target polypeptide, and allows the nucleic acid responsible for the alteration or modulation to be readily identified. The method may also utilize genomic libraries. While less desirable, the method may also utilize randomized nucleic acid sequences or a given sequence for which the function is not known, as described in, e.g., U.S. Pat. No. 5,639,595, the teaching of which is hereby incorporated by reference.

Accordingly, in one aspect, the invention features a method for identifying a nucleic acid associated with a modulation of a detectable phenotype in a cell, (e.g., a nucleic acid that modulates the function of a cell, that modulates expression of a target nucleic acid in a cell, or that modulates the biological activity of a target polypeptide in a cell.) The method involves (a) transforming a population of cells with a dsRNA expression library, where at least two cells of the population of cells are each transformed with a different nucleic acid from the dsRNA expression library, and where at least one encoded dsRNA specifically inhibits the expression of a target nucleic acid in at least one cell; (b) optionally selecting for a cell in which the nucleic acid is expressed in the cell; and (c) assaying for a modulation of a detectable phenotype of the cell, wherein detection of said modulation identifies a nucleic acid associated with the detectable phenotype of the cell. In a desirable embodiment, assaying for a modulation in the function of a cell involves measuring cell motility, apoptosis, cell growth, cell invasion, vascularization, cell cycle events, cell differentiation, cell dedifferentiation, neuronal cell regeneration, or the ability of a cell to support viral replication.

If desired, a short dsRNA or a nucleic acid (e.g., a vector) encoding a short dsRNA is administered to the cell to inhibit adverse effects due to the possible induction of the interferon response by the dsRNA expression library. See, e.g., U.S. Ser. No. 10/425,006 filed 28 Apr. 2003, “Methods of Silencing Genes Without Inducing Toxicity”, C. Pachuk, incorporated herein by reference. The short, second dsRNA differs from the dsRNA expression library and inhibits the interferon response or dsRNA-mediated toxicity. In some embodiments, the short, second dsRNA binds PKR and inhibits the dimerization and/or activation of PKR.

In some embodiments of these aspects, the short dsRNA and/or the long dsRNA is a forced or partial hairpin or a nucleic acid encoding a forced or partial hairpin, as described herein.

In one embodiment of any of the above aspects of the invention, in transforming step (a), the nucleic acid is stably integrated into a chromosome of the cell. Integration of the nucleic acid may be random or site-specific. Desirably integration is mediated by recombination or retroviral insertion. In addition, a single copy of the nucleic acid is desirably integrated into the chromosome and stably expressed. In another embodiment of any of the above aspects of the invention, in step (a) at least 50, more desirably 100; 500; 1000; 10,000; or 50,000 cells of the cell population are each transformed with a different nucleic acid from the dsRNA expression library. Desirably, the expression library is derived from the transfected cells or cells of the same cell type as the transfected cells. In other embodiments, the population of cells is transformed with at least 5%, more desirably at least 25%, 50%, 75%, or 90%, and most desirably at least 95% of the dsRNA expression library.

In other embodiments of any of the above aspects of the invention, the dsRNA expression library contains cDNA molecules or randomized nucleic acids. The dsRNA expression library may be a nuclear dsRNA expression library, in which case the double stranded nucleic acid is made in the nucleus. Alternatively, the dsRNA expression library may be a cytoplasmic dsRNA expression library, in which case the double stranded nucleic acid is made in the cytoplasm. In addition, the nucleic acid from the dsRNA expression library may be made in vitro or in vivo. In addition, the identified nucleic acid sequence may be located in the cytoplasm or nucleus of the cell.

In still another embodiment of any of the above aspects of the invention, the nucleic acid is contained in a vector, for example a dsRNA expression vector. The vector may then be transformed such that it is stably integrated into a chromosome of the cell, or it may function as an episomal (non-integrated) expression vector within the cell. In one embodiment, a vector that is integrated into a chromosome of the cell contains a promoter operably linked to a nucleic acid encoding a hairpin or dsRNA. In another embodiment, the vector does not contain a promoter operably linked to a nucleic acid encoding a dsRNA. In this latter embodiment, the vector integrates into a chromosome of a cell such that an endogenous promoter is operably linked to a nucleic acid from the vector that encodes a dsRNA.

Desirably, the dsRNA expression vector comprises at least one RNA polymerase II promoter, for example, a human CMV-immediate early promoter (HCMV-IE) or a simian CMV (SCMV) promoter, and/or at least one RNA polymerase I promoter, and/or at least one RNA polymerase III promoter. The promoter may also be a T7 promoter, in which case, the cell further comprises T7 polymerase. Alternatively, the promoter may be an SP6 promoter, in which case, the cell further comprises SP6 polymerase. The promoter may also be one convergent T7 promoter and one convergent SP6 promoter. A cell may be made to contain T7 or SP6 polymerase by transforming the cell with a T7 polymerase or an SP6 polymerase expression plasmid, respectively. In some embodiments, a T7 promoter or a RNA polymerase III promoter is operably linked to a nucleic acid that encodes a short dsRNA (e.g., a dsRNA that is less than 200, 150, 100, 75, 50, or 25 nucleotides in length). In other embodiments, the promoter is a mitochondrial promoter that allows cytoplasmic transcription of the nucleic acid in the vector (see, for example, the mitochondrial promoters described in WO 00/63364, filed Apr. 19, 2000). Alternatively, the promoter is an inducible promoter, such as a lac (Cronin et al. Genes & Development 15: 1506-1517, 2001), ara (Khlebnikov et al., J Bacteriol. 2000 December; 182(24):7029-34), ecdysone (Rheogene website), RU48 (mefepristone) (corticosteroid antagonist) (Wang X J, Liefer K M, Tsai S, O'Malley B W, Roop D R, Proc Natl Acad Sci USA. 1999 Jul. 20; 96(15):8483-8), or tet promoter (Rendal et al., Hum. Gene Ther. 2002; 13(2):335-42 and Larnartina et al., Hum. Gene Ther. 2002; 13(2):199-210) or a promoter disclosed in WO 00/63364, filed Apr. 19, 2000. In desirable embodiments, the inducible promoter is not induced until all the episomal vectors are eliminated from the cell. The vector may also comprise a selectable marker.

In particular embodiments, the dsRNA encoded by the dsRNA expression library is between 11 and 40 nucleotides in length and, in the absence of short dsRNA of the invention, induces toxicity in vertebrate cells because its sequence has affinity for PKR or another protein in a dsRNA-mediated stress response pathway. The short dsRNA of the invention inhibits this toxicity.

In still other embodiments of any of the above aspects of the invention, the cell and the vector each further comprise a loxP site and site-specific integration of the nucleic acid into a chromosome of the cell occurs through recombination between the loxP sites. In addition, the method further involves rescuing the nucleic acid through Cre-mediated double recombination.

In yet another embodiment of any of the above aspects of the invention, the cell is derived from a parent cell, and is generated by (a) transforming a population of parent cells with a bicistronic plasmid expressing a selectable marker and a reporter gene, and comprising a loxP site; (b) selecting for a cell in which the plasmid is stably integrated; and (c) selecting for a cell in which one copy of the plasmid is stably integrated in a transcriptionally active locus. Desirably the selectable marker is G418 and the reporter gene is green fluorescent protein (GFP). These methods are disclosed in further detail in U.S. Published Application 2002/0132257 and European Published Application 1229134, “Use of post-transcriptional gene silencing for identifying nucleic acid sequences that modulate the function of a cell”, the teaching of which is hereby incorporated by reference.

Methods for Identifying Nucleic Acids of Interest by Transfecting Cells with dsRNA Molecules

In addition to the above screening methods that utilize a dsRNA expression library, the invention provides screening methods that utilize one or more dsRNA molecules with substantial sequence identity to a target gene to inhibit expression of the target gene. If desired, one or more short dsRNA molecules can also be administered to inhibit the interferon response. Desirably, the method is carried out under conditions that inhibit or prevent an interferon response or dsRNA stress response.

In one such aspect, the invention features a method for identifying a nucleic acid that modulates a detectable phenotype in a cell, (e.g., a nucleic acid that modulates the function of a cell, that modulates expression of a target nucleic acid in a cell, or that modulates the biological activity of a target polypeptide in a cell.) involving (a) transforming a population of cells with a first dsRNA; (b) optionally selecting for a cell in which the nucleic acid is expressed; and (c) assaying for a modulation in the detectable phenotype of the cell. The first dsRNA has substantial sequence identity to a target nucleic acid in the cell and specifically inhibits the expression of the target nucleic acid. In a desirable embodiment, the target nucleic to acid is assayed using DNA array technology. In a desirable embodiment, assaying for a modulation in the function of a cell involves measuring cell motility, apoptosis, cell growth, cell invasion, vascularization, cell cycle events, cell differentiation, cell dedifferentiation, neuronal cell regeneration, or the ability of a cell to support viral replication.

In various embodiments of the above aspect, either a short, second dsRNA or a nucleic acid encoding a short, second dsRNA is also administered to the cells. The short, second dsRNA differs from the first dsRNA and inhibits the interferon response or dsRNA-mediated toxicity. In some embodiments, the short, second dsRNA binds PKR and inhibits the dimerization and/or activation of PKR. See the teaching of U.S. Ser. No. 10/425,006 filed 28 Apr. 2003, “Methods of Silencing Genes Without Inducing Toxicity”, C. Pachuk, incorporated herein by reference. In some embodiments of the above aspects, the short dsRNA and/or the long dsRNA is a forced or partial hairpin or a nucleic acid encoding a forced or partial hairpin as described herein.

### Additional Embodiments of any of the Various Aspects of the Invention

In one embodiment of any of the above aspects of the invention, at least 2, more desirably 50; 100; 500; 1000; 10,000; or 50,000 cells of the population of cells are each transformed with a different dsRNA. Desirably, at most one first dsRNA (e.g., one long dsRNA) is inserted into each cell. In other embodiments, the population of cells is transformed with at least 5%, more desirably at least 25%, 50%, 75%, or 90%, and most desirably, at least 95% of the dsRNA expression library or dsRNA library. In still another embodiment, the method further involves identifying the nucleic acid sequence by amplifying and cloning the sequence. Desirably amplification of the sequence involves the use of the polymerase chain reaction (PCR).

Desirable Vectors

In still another embodiment of any of the various aspects of the invention, the nucleic acid is contained in a vector, for example, a dsRNA expression vector that is capable of forming a dsRNA. Desirably the dsRNA expression vector comprises at least one promoter. The promoter may be a T7 promoter, in which case, the cell further comprises T7 polymerase. Alternatively, the promoter may be an SP6 promoter, in which case, the cell further comprises SP6 polymerase. The promoter may also be one convergent T7 promoter and one convergent SP6 promoter. A cell may be made to contain T7 or SP6 polymerase by transforming the cell with a T7 polymerase or an SP6 polymerase expression plasmid, respectively. The vector may also comprise a selectable marker, for example hygromycin. In some embodiments, the same vector encodes the dsRNA and the polymerase (e.g., a T7 or SP6 polymerase).

Desirably, in a vector for use in the methods of the invention, the sense strand and the antisense strand of the nucleic acid sequence are transcribed from the same nucleic acid sequence using two convergent promoters. In another desirable embodiment, in a vector for use in any of the above aspects of the invention, the nucleic acid sequence comprises an inverted repeat, such that upon transcription, the nucleic acid forms a dsRNA. In desirable embodiments, the dsRNA is a forced or partial hairpin or a nucleic acid encoding a forced or partial hairpin as described herein.

Other desirable vectors have an origin of replication that enables the DNA vector to be replicated upon nuclear localization, such as the SV40 T origin, EBNA origin, or a mammalian origin. Desirably, the vector with the origin of replication or another vector or chromosome in the cell encodes an accessory factor such as SV40 TAg or EBNA that enables the vector to replicate in the cell.

Desirable dsRNA Molecules

Desirable methods of any of the above aspects use one or more dsRNA molecules (e.g., full or partial hairpins), or one or more vectors of the invention. In some embodiments, the dsRNA contains coding sequence, non-coding sequence, or a combination thereof. For TGS applications, the dsRNA desirably includes a regulatory sequence (e.g., a transcription factor binding site, a promoter, and/or a 5′ or 3′ untranslated region (UTR) of an mRNA) and/or a coding sequence. For PTGS applications, the dsRNA desirably includes a regulatory sequence (e.g., a 5′ or 3′ untranslated region (UTR) of an mRNA) and/or a coding sequence. In some embodiments, the same dsRNA mediates both TGS and PTGS. In other embodiments, one or more dsRNA molecules that mediate TGS and one or more dsRNA molecules that mediate PTGS are used. In some embodiments, the dsRNA has a 1, 2, 3, 4, 5, 6, or more constitutive transport element (CTE) sequences (e.g., a CTE from Mason-Pfizer Monkey virus). In certain embodiments, the dsRNA has one or more introns and/or a polyA tail. Desirably, the amount of dsRNA located in the cytoplasm of a cell is at least 24, 50, 75, 100, 200, 400, 600, or even 1000% greater for a dsRNA that has a CTE, intron, and/or polyA tail than for a control dsRNA lacking the CTE, intron, and/or polyA tail.

In other embodiments of any of the above aspects of the invention, the short or long dsRNA is derived from cDNA molecules or randomized nucleic acids. In some embodiments, the dsRNA is located in the cytoplasm or nucleus. In some embodiments, some of the dsRNA transcripts are located in the cytoplasm, and some of the transcripts are located in the nucleus. Desirably, the dsRNA mediates both PTGS and TGS. In other embodiments, at least 50, 60, 70, 80, 90, 95, or 100% of the dsRNA molecules are located in the cytoplasm and thus can mediate PTGS. In still other embodiments, at least 50, 60, 70, 80, 90, 95, or 100% of the dsRNA molecules are located in the nucleus and can mediate TGS. In some embodiments, dsRNA molecules that mediate TGS comprise a region substantially identical to the promoter of a target gene. Other dsRNA molecules have, e.g., a region substantially identical to the promoter and a region substantially identical to the coding region of the target gene. The dsRNA may be made in vitro or in vivo. In various embodiments, the identified nucleic acid sequence is located in the cytoplasm or nucleus of the cell.

In yet another embodiment, the dsRNA is at least 100, 500, 600, or 1000 nucleotides in length. In other embodiments, the dsRNA is at least 10, 20, 30, 40, 50, 60, 70, 80, or 90 nucleotides in length. In yet other embodiments, the number of nucleotides in the dsRNA is between 5-100 nucleotides, 15-100 nucleotides, 20-95 nucleotides, 25-90 nucleotides, 35-85 nucleotides, 45-80 nucleotides, 50-75 nucleotides, or 55-70 nucleotides, inclusive. In still other embodiments, the number of nucleotides in the dsRNA is contained in one of the following ranges: 5-15 nucleotides, 15-20 nucleotides, 19-26 nucleotides, 20-25 nucleotides, 25-35 nucleotides, 35-45 nucleotides, 45-60 nucleotides, 60-70 nucleotides, 70-80 nucleotides, 80-90 nucleotides, or 90-100 nucleotides, inclusive. In other embodiments, the dsRNA contains less than 50,000; 10,000; 5,000; or 2,000 nucleotides. In addition, the dsRNA may contain a sequence that is less than a full length RNA sequence. In other desirable embodiments, the double stranded region in the dsRNA (e.g., a long dsRNA) contains between 11 and 30 nucleotides, inclusive; between 19 and 26 nucleotides, inclusive; over 30 nucleotides; or over 200 nucleotides. In desirable embodiments, the double stranded region in the short dsRNA contains between 11 and 30 nucleotides, inclusive; or between 19 and 26 nucleotides, inclusive.

In some embodiments, the dsRNA (e.g., the first dsRNA) is 20 to 30 nucleotides (e.g., 20, 21, 22, 23, 24, 25, 26, 27, or 28 nucleotides) in length. In particular embodiments, the first dsRNA is between 11 and 40 nucleotides in length and, in the absence of short dsRNA of the invention, induces toxicity in vertebrate cells because its sequence has affinity for PKR or another protein in a dsRNA mediated stress response pathway. The short dsRNA of the invention inhibits this toxicity.

In other embodiments, the dsRNA is derived from a cell or a population of cells and is used to transform another cell population of either the same cell type or a different cell type. In desirable embodiments, the transformed cell population contains cells of a cell type that is related to the cell type of the cells from which the dsRNA was derived (e.g., the transformation of cells of one neuronal cell type with the dsRNA derived from cells of another neuronal cell type). In yet other embodiments of any of these aspects, the dsRNA contains one or more contiguous or non-contiguous positions that are randomized (e.g., by chemical or enzymatic synthesis using a mixture of nucleotides that may be added at the randomized position). In still other embodiments, the dsRNA is a randomized nucleic acid in which segments of ribonucleotides and/or deoxyribonucleotides are ligated to form the dsRNA. In desirable embodiments, the agent, nucleic acid, dsRNA, or vector is a nucleic acid of the invention (e.g., a partial or full hairpin, or a vector encoding a partial or full hairpin).

In other embodiments of any of the various aspects of the invention, the dsRNA (e.g., a long dsRNA) specifically hybridizes to a target nucleic acid but does not substantially hybridize to non-target molecules, which include other nucleic acids in the cell or biological sample having a sequence that is less than 99, 95, 90, 80, or 70% identical or complementary to that of the target nucleic acid. Desirably, the amount of the non-target molecules hybridized to, or associated with, the dsRNA, as measured using standard assays, is 2-fold, desirably 5-fold, more desirably 10-fold, and most desirably 50-fold lower than the amount of the target nucleic acid hybridized to, or associated with, the dsRNA. In other embodiments, the amount of a target nucleic acid hybridized to, or associated with, the dsRNA, as measured using standard assays, is 2-fold, desirably 5-fold, more desirably 10-fold, and most desirably 50-fold greater than the amount of a control nucleic acid hybridized to, or associated with, the dsRNA. Desirably, the dsRNA (e.g., a long dsRNA) only hybridizes to one target nucleic acid from a cell under denaturing, high stringency hybridization conditions. In certain embodiments, the dsRNA is substantially homologous (e.g., at least 80, 90, 95, 98, or 100% homologous) to only one target nucleic acid from a cell.

In other embodiments, the dsRNA is homologous to multiple RNA molecules, such as RNA molecules from the same gene family. In yet other embodiments, the dsRNA is homologous to distinctly different mRNA sequences from genes that are similarly regulated (e.g., developmental, chromatin remodeling, or stress response induced). In other embodiments, the dsRNA is homologous to a large number of RNA molecules, such as a dsRNA designed to induce a stress response or apoptosis (e.g., a dsRNA designed to kill cancer cells or other unhealthy or excess cells).

In other embodiments, the percent decrease in the expression of a target nucleic acid is at least 2, 5, 10, 20, or 50 fold greater than the percent decrease in the expression of a non-target or control nucleic acid. Desirably, the dsRNA inhibits the expression of a target nucleic acid but has negligible, if any, effect on the expression of other nucleic acids in the cell. Examples of control nucleic acids include nucleic acids with a random sequence or nucleic acids known to have little, if any, affinity for the dsRNA. Desirably, the long and short dsRNA molecules (e.g., long and short dsRNA) are substantially non-homologous to a naturally-occurring essential mammalian gene or to all the essential mammalian genes (see, for example, WO 00/63364). In some embodiments, the dsRNA does not adversely affect the function of an essential gene. In other embodiments, the dsRNA adversely affects the function of an essential gene in a cancer cell.

Desirably, the short dsRNA inhibits the dimerization of PKR or another protein in a dsRNA-mediated stress response pathway by at least 10, 20, 30, 40, 50, 60, 70, 80, 90, or 95% compared to the amount of dimerization of the protein in a control cell or animal not administered the short dsRNA, as measured using standard methods such as those described herein. In some embodiments, the short dsRNA includes a region of randomized sequence, or the entire short dsRNA contains randomized sequence. In various embodiments, the short dsRNA does not substantially decrease the expression of a nucleic acid in the cell (e.g., decreases expression by less than 60, 40, 30, 20, or 10%). In certain embodiments, the sequence of the short dsRNA is less than 80, 70, 60, 50, 30, 20, or 10% identical to or complementary to that of a nucleic acid in the cell. In particular embodiments, multiple short dsRNA molecules or multiple vectors encoding short dsRNA are administered to the cell and less than 70, 60, 50, 30, 20, or 10% of the short dsRNA molecules have a sequence that is at least 50, 70, 80, or 90% identical to or complementary to that of a nucleic acid in the cell.

In other embodiments of any of various aspects of the invention, at most one molecular species of long dsRNA is inserted into each cell. In other embodiments, at most one vector encoding a long dsRNA is stably integrated into the genome of each cell and one dsRNA stably expressed therefrom. In various embodiments, the dsRNA is active in the nucleus of the transformed cell and/or is active in the cytoplasm of the transformed cell. In various embodiments, at least 1, 10, 20, 50, 100, 500, or 1000 cells or all of the cells in the population are selected as cells that contain or express a dsRNA (e.g., a long dsRNA). In some embodiments, at least 1, 10, 20, 50, 100, 500, or 1000 cells or all of the cells in the population are assayed for a modulation of a detectable phenotype, e.g., modulation in the function of the cell, a modulation in the expression of a target nucleic acid (e.g., an endogenous or pathogen gene) in the cell, and/or a modulation in the biological activity of a target protein (e.g., an endogenous or pathogen protein) in the cell.

Desirable RNA Polymerases

In certain embodiments, an RNA dependent-RNA polymerase is expressed in a cell or animal into which a dsRNA or a vector encoding a dsRNA is introduced. The RNA dependent-RNA polymerase amplifies the dsRNA and desirably increases the number of dsRNA molecules in the cell or animal by at least 25, 50, 100, 200, 500, 1000, 5000, or even 10000%. In various embodiments, the RNA dependent-RNA polymerase is naturally expressed by the cell or animal or is encoded by the same or a different vector that encodes the dsRNA. Exemplary RNA dependent-RNA polymerases include viral, plant, invertebrate, or vertebrate (e.g., mammalian or human) RNA dependent-RNA polymerases. Providing an RNA dependent-RNA polymerase (RdRp) is especially important in those embodiments of the invention that utilize partial hairpin dsRNAs which are extended in vitro or in vivo with an RNA dependent-RNA polymerase, unless the cells or system in which the partial hairpin is utilized contains an endogenous RdRp. See Table 1, which provides a non-exclusive list of RNA dependent-RNA polymerases useful in the methods of the invention.

TABLE 1
RNA dependent RNA polymerases

| | Genbank |
|---|---|
| Source of Polymerase | Accession No. |
| Turnip Crinkle Virus | NC_003821 |
| Taura Syndrome Virus | NC_003005 |
| L. esculentum mRNA for RNA-directed RNA | Y10403 |
| polymerase | |
| Perina Nuda Picorna-like Virus | NC_003113 |
| Dengue Virus Type 2 Strain TSV01 | AY037116 |
| Caenorhabditis elegans RNA-directed RNA | AF159143 |
| polymerase related EGO-1 (ego-1) mRNA | |
| Caenorhabditis elegans RRF-1 (rrf-1) mRNA | AF159144 |
| Hepatitis C virus | NC_001433 |
| Cucumber Leaf Spot Virus putative RNA- | AY038365 |
| dependent RNA polymerase gene | |
| Pseudomonas phage phi-6 segment M | NC_003716 |
| Pseudomonas phage phi-6 segment L | NC_003715 |
| Pseudomonas phage phi-6 segment S | NC_003714 |
| Chain P, RNA Dependent RNA Polymerase from | 1HI0P |
| dsRNA Bacteriophage Phi6 Plus Initiation | |
| Complex | |
| Bovine Viral Diarrhea Virus Genotype 2 | NC_002032 |
| Putative Polyprotein [Bovine Viral Diarrhea Virus | NP_044731 |
| Genotype 2] | |

Optional Administration of Target Gene

In some embodiments, a target gene (e.g., a pathogen or endogenous target gene) or a region from a target gene (e.g., a region from an intron, exon, untranslated region, promoter, or coding region) is introduced into the cell or animal. For example, this target nucleic acid can be inserted into a vector (e.g., a vector that desirably can integrate into the genome of a cell) and then administered to the cell or animal. Desirably, the administration of one or more copies of the target nucleic acid enhances the amplification of the dsRNA that is homologous to the target nucleic acid or enhances the amplification of cleavage products from this dsRNA.

Optional Methods to Inhibit Interferon Response

In some embodiments, a component of the interferon response or dsRNA stress response pathway (e.g., PKR, human beta interferon, and/or 2′5′OAS) is inhibited in the cell or animal. In various embodiments, one or more components are inhibited using dsRNA-mediated gene silencing, antisense-mediated gene silencing, ribozyme-mediated gene silencing, or genetic knockout methods. Additionally, one or more IRE sequences and/or one or more transcription factors that bind IRE sequences, such as STAT1, can be optionally silenced or mutated. In various embodiments, one or more nucleic acids that encode proteins that block the PKR response, such as the Vaccinia virus protein E3, the cellular protein P58^{IPK}, or a Hepatitis C E2 protein, are administered to the cell or animal.

Desirable Methods of Administration of Nucleic Acids

In some embodiments, the dsRNA or dsRNA expression vector is complexed with one or more cationic lipids or cationic amphiphiles, such as the compositions disclosed in U.S. Pat. No. 4,897,355 (Eppstein et al., filed Oct. 29, 1987), U.S. Pat. No. 5,264,618 (Feigner et al., filed Apr. 16, 1991) or U.S. Pat. No. 5,459,127 (Felgner et al., filed Sep. 16, 1993). In other embodiments, the dsRNA or dsRNA expression vector is complexed with a liposome/liposomic composition that includes a cationic lipid and optionally includes another component, such as a neutral lipid (see, for example, U.S. Pat. No. 5,279,833 (Rose), U.S. Pat. No. 5,283,185 (Epand), and U.S. Pat. No. 5,932,241 (Gorman)). In other embodiments, the dsRNAs or dsRNA expression constructs are complexed with the multifunctional molecular complexes of U.S. Pat. No. 5,837,533, U.S. Pat. No. 6,127,170, and U.S. Pat. No. 6,379,965 (Boutin), or the multifunctional molecular complexes or oil/water cationic amphiphile emulsions of PCT/US03/14288, filed May 6, 2003 (Satishchandran).

In yet other embodiments, the dsRNA or dsRNA expression vector is complexed with any other composition that is devised by one of ordinary skill in the fields of pharmaceutics and molecular biology. In some embodiments, the dsRNA or the vector is not complexed with a cationic lipid.

Transformation/transfection of the cell may occur through a variety of means including, but not limited to, lipofection, DEAE-dextran-mediated transfection, microinjection, protoplast fusion, calcium phosphate precipitation, viral or retroviral delivery, electroporation, or biolistic transformation. The RNA or RNA expression vector (DNA) may be naked RNA or DNA or local anesthetic complexed RNA or DNA (Pachuk et al., supra). In yet another embodiment, the cell is not a C. elegans cell. Desirably the vertebrate (e.g., mammalian) cell has been cultured for only a small number of passages (e.g., less than 30 passages of a cell line that has been directly obtained from American Type Culture Collection), or are primary cells. In addition, desirably the vertebrate (e.g., mammalian) cell is transformed with dsRNA that is not complexed with cationic lipids.

Desirable Cells

In still further embodiments of any aspect of the invention, the cell is a plant cell or an animal cell. Desirably the animal cell is an invertebrate or vertebrate cell (e.g., a mammalian cell, for example, a human cell). The cell may be ex vivo or in vivo. The cell may be a gamete or a somatic cell, for example, a cancer cell, a stem cell, a cell of the immune system, a neuronal cell, a muscle cell, or an adipocyte. In some embodiments, one or more proteins involved in gene silencing, such as Dicer or Argonaut, are overexpressed or activated in the cell or animal to increase the amount of inhibition of gene expression.

Some Advantages of the Present Invention

The present methods provide numerous advantages for the silencing of genes in cells and animals. For example, in other dsRNA delivery systems some dsRNA molecules induce an interferon response (Jaramillo et al., Cancer Invest. 13:327-338, 1995). Induction of an interferon response is not desired because it can lead to cell death and possibly prevent gene silencing. Thus, a significant advantage of the present invention is that the dsRNA delivery methods described herein are performed such that an interferon response is inhibited or prevented. These methods allow dsRNA to be used in clinical applications for the prevention or treatment of disease or infection without the generation of adverse side-effects due to dsRNA-induced toxicity. The use of both short and long dsRNA molecules in some embodiments of the present methods may also have improved efficiency for silencing genes, as compared to previous methods that use only short dsRNA molecules.

### DEFINITIONS

By “agent that provides an at least partially double-stranded RNA” is meant a composition that generates an at least partially double-stranded (ds)RNA in a cell or animal. For example, the agent can be a dsRNA, a single stranded RNA molecule that assumes a double stranded conformation inside the cell or animal (e.g., a hairpin), or a combination of two single stranded RNA molecules that are administered simultaneously or sequentially and that assume a double stranded conformation inside the cell or animal. Other exemplary agents include a DNA molecule, plasmid, viral vector, or recombinant virus encoding an at least partially dsRNA. Other agents are disclosed in WO 00/63364, filed Apr. 19, 2000. In some embodiments, the agent includes between 1 ng and 20 mg, 1 ng to 1 ug, 1 ug to 1 mg, or 1 mg to 20 mg of DNA and/or RNA.

By “alteration in the level of gene expression” is meant a change in transcription, translation, or mRNA or protein stability, such that the overall amount of a product of the gene, i.e., mRNA or polypeptide, is increased or decreased.

By “apoptosis” is meant a cell death pathway wherein a dying cell displays a set of well-characterized biochemical hallmarks that include cytolemmal membrane blebbing, cell soma shrinkage, chromatin condensation, nuclear disintegration, and DNA laddering. There are many well-known assays for determining the apoptotic state of a cell, including, and not limited to: reduction of MTT tetrazolium dye, TUNEL staining, Annexin V staining, propidium iodide staining, DNA laddering, PARP cleavage, caspase activation, and assessment of cellular and nuclear morphology. Any of these or other known assays may be used in the methods of the invention to determine whether a cell is undergoing apoptosis.

By “assaying” is meant analyzing the effect of a treatment, be it chemical or physical, administered to whole animals, cells, tissues, or molecules derived therefrom. The material being analyzed may be an animal, a cell, a tissue, a lysate or extract derived from a cell, or a molecule derived from a cell. The analysis may be, for example, for the purpose of detecting altered cell function, altered gene expression, altered endogenous RNA stability, altered polypeptide stability, altered polypeptide levels, or altered polypeptide biological activity. The means for analyzing may include, for example, antibody labeling, immunoprecipitation, phosphorylation assays, glycosylation assays, and methods known to those skilled in the art for detecting nucleic acid molecules. In some embodiments, assaying is conducted under selective conditions.

By “Cre-mediated double recombination” is meant two nucleic acid recombination events involving loxP sites that are mediated by Cre recombinase. A Cre-mediated double recombination event can occur, for example, as disclosed in more detail in U.S. Published Application 2002/0132257, and, e.g., in FIG. 1 thereof.

By “bacterial infection” is meant the invasion of a host animal by pathogenic bacteria. For example, the infection may include the excessive growth of bacteria that are normally present in or on the body of a animal or growth of bacteria that are not normally present in or on the animal. More generally, a bacterial infection can be any situation in which the presence of a bacterial population(s) is damaging to a host animal. Thus, a animal is “suffering” from a bacterial infection when an excessive amount of a bacterial population is present in or on the animal's body, or when the presence of a bacterial population(s) is damaging the cells or other tissue of the animal. In one embodiment, the number of a particular genus or species of bacteria is at least 2, 4, 6, or 8 times the number normally found in the animal. The bacterial infection may be due to gram positive and/or gram negative bacteria.

By “a decrease” is meant a lowering in the level of: a) protein (e.g., as measured by ELISA or Western blot analysis); b) reporter gene activity (e.g., as measured by reporter gene assay, for example, β-galactosidase, green fluorescent protein, or luciferase activity); c) mRNA (e.g., as measured by RT-PCR or Northern blot analysis relative to an internal control, such as a “housekeeping” gene product, for example, β-actin or glyceraldehyde 3-phosphate dehydrogenase (GAPDH)); or d) cell function, for example, as assayed by the number of apoptotic, mobile, growing, cell cycle arrested, invasive, differentiated, or dedifferentiated cells in a test sample. In all cases, the lowering is desirably by at least 20%, more desirably by at least 30%, 40%, 50%, 60%, 75%, and most desirably by at least 90%. As used herein, a decrease may be the direct or indirect result of PTGS, TGS, or another gene silencing event.

By “nucleic acid molecule” is meant a compound in which one or more molecules of phosphoric acid are combined with a carbohydrate (e.g., pentose or hexose) which are in turn combined with bases derived from purine (e.g., adenine or guanine) and from pyrimidine (e.g., thymine, cytosine, or uracil). Particular naturally-occurring nucleic acid molecules include genomic deoxyribonucleic acid (DNA) and genomic ribonucleic acid (RNA), as well as the several different forms of the latter, e.g., messenger RNA (mRNA), transfer RNA (tRNA), and ribosomal RNA (rRNA). Also included are different DNA molecules which are complementary (cDNA) to the different RNA molecules. Synthesized DNA, or a hybrid thereof with naturally-occurring DNA, as well as DNA/RNA hybrids, and PNA molecules (Gambari, Curr Pharm Des 2001 November; 7(17):1839-62) are also included within the definition of “nucleic acid molecule.”

Nucleic acids typically have a sequence of two or more covalently bonded naturally-occurring or modified deoxyribonucleotides or ribonucleotides. Modified nucleic acids include, e.g., peptide nucleic acids and nucleotides with unnatural bases. Modifications include those chemical and structural modifications described under the definition of “dsRNA” below. Also included are, e.g., various structures, as described within the definitions of “dsRNA”, “expression vectors”, and “expression constructs”, and elsewhere in this specification.

By “dsRNA” is meant a nucleic acid molecule containing a region of two or more nucleotides that are in a double stranded conformation. In various embodiments, the dsRNA consists entirely of ribonucleotides or consists of a mixture of ribonucleotides and deoxynucleotides, such as the RNA/DNA hybrids disclosed, for example, by WO 00/63364, filed Apr. 19, 2000, or U.S. Ser. No. 60/130,377, filed Apr. 21, 1999. The dsRNA may be a single molecule with regions of self-complimentarily such that nucleotides in one segment of the molecule base pair with nucleotides in another segment of the molecule. In various embodiments, a dsRNA that consists of a single molecule consists entirely of ribonucleotides or includes a region of ribonucleotides that is complimentary to a region of deoxyribonucleotides. Alternatively, the dsRNA may include two different strands that have a region of complimentarily to each other. In various embodiments, both strands consist entirely of ribonucleotides, one strand consists entirely of ribonucleotides and one strand consists entirely of deoxyribonucleotides, or one or both strands contain a mixture of ribonucleotides and deoxyribonucleotides. Desirably, the regions of complimentarily are at least 70, 80, 90, 95, 98, or 100% complimentary. Desirably, the region of the dsRNA that is present in a double stranded conformation includes at least 19, 20, 30, 50, 75, 100, 200, 500, 1000, 2000, or 5000 nucleotides, or includes all of the nucleotides in a cDNA being represented in the dsRNA. In some embodiments, the dsRNA does not contain any single stranded regions, such as single stranded ends, or the dsRNA is a hairpin. In other embodiments, the dsRNA has one or more single stranded regions or overhangs. Desirable RNA/DNA hybrids include a DNA strand or region that is an antisense strand or region (e.g., has at least 70, 80, 90, 95, 98, or 100% complimentarily to a target nucleic acid) and an RNA strand or region that is a sense strand or region (e.g., has at least 70, 80, 90, 95, 98, or 100% identity to a target nucleic acid), or vice versa. In various embodiments, the RNA/DNA hybrid is made in vitro using enzymatic or chemical synthetic methods such as those described herein, or those described in WO 00/63364, filed Apr. 19, 2000 or U.S. Ser. No. 60/130,377, filed Apr. 21, 1999. In other embodiments, a DNA strand synthesized in vitro is complexed with an RNA strand made in vivo or in vitro before, after, or concurrent with the transformation of the DNA strand into the cell. In yet other embodiments, the dsRNA is a single circular nucleic acid containing a sense and an antisense region, or the dsRNA includes a circular nucleic acid and either a second circular nucleic acid or a linear nucleic acid (see, for example, WO 00/63364, filed Apr. 19, 2000 or U.S. Ser. No. 60/130,377, filed Apr. 21, 1999). Exemplary circular nucleic acids include lariat structures in which the free 5′ phosphoryl group of a nucleotide becomes linked to the 2′ hydroxyl group of another nucleotide in a loop back fashion.

In other embodiments, the dsRNA includes one or more modified nucleotides in which the 2′ position in the sugar contains a halogen (such as flourine group) or contains an alkoxy group (such as a methoxy group) which increases the half-life of the dsRNA in vitro or in vivo compared to the corresponding dsRNA in which the corresponding 2′ position contains a hydrogen or an hydroxyl group. In yet other embodiments, the dsRNA includes one or more linkages between adjacent nucleotides other than a naturally-occurring phosphodiester linkage. Examples of such linkages include phosphoramide, phosphorothioate, and phosphorodithioate linkages. In other embodiments, the dsRNA contains one or two capped strands or no capped strands, as disclosed, for example, by WO 00/63364, filed Apr. 19, 2000 or U.S. Ser. No. 60/130,377, filed Apr. 21, 1999. In other embodiments, the dsRNA contains coding sequence or non-coding sequence, for example, a regulatory sequence (e.g., a transcription factor binding site, a promoter, or a 5′ or 3′ untranslated region (UTR) of an mRNA). Additionally, the dsRNA can be any of the at least partially double-stranded RNA molecules disclosed in WO 00/63364, filed Apr. 19, 2000 (see, for example, pages 8-22). Any of the dsRNA molecules may be expressed in vitro or in vivo using the methods described herein, or using standard methods, such as those described in WO 00/63364, filed Apr. 19, 2000 (see, for example, pages 16-22).

By “dsRNA expression library” is meant a collection of nucleic acid expression vectors containing nucleic acid sequences, for example, cDNA sequences or randomized nucleic acid sequences that are capable of forming a dsRNA (dsRNA) upon expression of the nucleic acid sequence. Desirably the dsRNA expression library contains at least 10,000 unique nucleic acid sequences, more desirably at least 50,000; 100,000; or 500,000 unique nucleic acid sequences, and most desirably, at least 1,000,000 unique nucleic acid sequences. By a “unique nucleic acid sequence” is meant that a nucleic acid sequence of a dsRNA expression library has desirably less than 50%, more desirably less than 25% or 20%, and most desirably less than 10% nucleic acid identity to another nucleic acid sequence of a dsRNA expression library when the full length sequence is compared. Sequence identity is typically measured using BLAST® (Basic Local Alignment Search Tool) or BLAST® 2 with the default parameters specified therein (see, Altschul et al., J. Mol. Biol. 215:403-410 (1990); and Tatiana et al., FEMS Microbiol. Lett. 174:247-250 (1999)). This software program matches similar sequences by assigning degrees of homology to various substitutions, deletions, and other modifications. Conservative substitutions typically include substitutions within the following groups: glycine, alanine, valine, isoleucine, leucine; aspartic acid, glutamic acid, asparagine, glutamine; serine, threonine; lysine, arginine; and phenylalanine, tyrosine.

The preparation of cDNAs for the generation of dsRNA expression libraries is described, e.g., in U.S. Published Application 2002/0132257 and European Published Application 1229134, “Use of post-transcriptional gene silencing for identifying nucleic acid sequences that modulate the function of a cell”, the teaching of which is hereby incorporated by reference. A randomized nucleic acid library may also be generated as described, e.g., in U.S. Pat. No. 5,639,595, the teaching of which is hereby incorporated by reference, and utilized for dsRNA-mediated functional genomics applications. The dsRNA expression library may contain nucleic acid sequences that are transcribed in the nucleus or that are transcribed in the cytoplasm of the cell. A dsRNA expression library may be generated using techniques described herein.

By an “expression construct” is meant any double-stranded DNA or double-stranded RNA designed to transcribe an RNA, e.g., a construct that contains at least one promoter operably linked to a downstream gene or coding region of interest (e.g., a cDNA or genomic DNA fragment that encodes a protein, or any RNA of interest). Transfection or transformation of the expression construct into a recipient cell allows the cell to express RNA or protein encoded by the expression construct. An expression construct may be a genetically engineered plasmid, virus, or an artificial chromosome derived from, for example, a bacteriophage, adenovirus, retrovirus, poxvirus, or herpesvirus. An expression construct can be replicated in a living cell, or it can be made synthetically.

By “expression vector” is meant a DNA construct that contains at least one promoter operably linked to a downstream gene or coding region (e.g., a cDNA or genomic DNA fragment that encodes a protein, optionally, operatively linked to sequence lying outside a coding region, an antisense RNA coding region, or RNA sequences lying outside a coding region). Transfection or transformation of the expression vector into a recipient cell allows the cell to express RNA encoded by the expression vector. An expression vector may be a genetically engineered plasmid, virus, or artificial chromosome derived from, for example, a bacteriophage, adenovirus, retrovirus, poxvirus, or herpesvirus.

By “forced hairpin” is meant a nucleic acid molecule (e.g., a DNA molecule or vector) or a population of nucleic acid molecules encoding an RNA (e.g., a partial or full hairpin) that has, in 5′ to 3′ order, a first region of interest, a first base-paired region, a loop region, and a second base-paired region. The first and second base-paired regions are base-paired to each other. Desirably, the nucleic acid further includes a second region of interest downstream of the second base-paired region. If the second region of interest is present, the first and second regions of interest are base-paired to each other. Desirably, at least 50, 60, 70, 80, 90, 95, or 100% of the nucleotides in first and second regions of interest participate in Watson-Crick base-pairing with each other. These two regions between may be the same length or may differ in length by one or more nucleotides. For example, one region of interest may have additional nucleotides at one end of the region that are not base-paired to nucleotides in any portion of the other region of interest. In a related aspect, the invention features an RNA molecule or a population of RNA molecules encoded by these nucleic acids. Exemplary RNA molecules are illustrated in FIGS. 1A-1D, in which the first and second base-paired regions are denoted “A” and “B,” and the first and second regions of interest are respectively denoted “sense or antisense with respect to the target” or “antisense or sense with respect to the target.” In each case, if the first region is a sense sequence with respect to the target gene, the second region of interest will be antisense with respect to the target, and vice versa, so that the two regions are inversely complementary.

By “full RNA hairpin” is meant a hairpin without a single stranded overhang.

By “function of a cell” is meant any cell activity that can be measured or assessed. Examples of cell function include, but are not limited to, cell motility, apoptosis, cell growth, cell invasion, vascularization, cell cycle events, cell differentiation, cell dedifferentiation, neuronal cell regeneration, and the ability of a cell to support viral replication. The function of a cell may also be to affect the function, gene expression, or the polypeptide biological activity of another cell, for example, a neighboring cell, a cell that is contacted with the cell, or a cell that is contacted with media or other extracellular fluid in which the cell is contained.

By “high stringency conditions” is meant hybridization in 2×SSC at 40° C. with a DNA probe length of at least 40 nucleotides. For other definitions of high stringency conditions, see F. Ausubel et al., Current Protocols in Molecular Biology, pp. 6.3.1-6.3.6, John Wiley & Sons, New York, N.Y., 1994, hereby incorporated by reference.

By “isolated nucleic acid,” “nucleic acid sequence,” “nucleic acid molecule,” “dsRNA nucleic acid sequence,” or “dsRNA nucleic acid” is meant a nucleic acid molecule, or a portion thereof, that is free of the genes that, in the naturally-occurring genome of the organism from which the nucleic acid sequence of the invention is derived, flank the gene. The term therefore includes, for example, a recombinant DNA, with or without 5′ or 3′ flanking sequences that is incorporated into a vector, for example, dsRNA expression vector; into an autonomously replicating plasmid or virus; or into the genomic DNA of a prokaryote or eukaryote; or which exists as a separate molecule (e.g., a cDNA or a genomic or cDNA fragment produced by PCR or restriction endonuclease digestion) independent of other sequences.

By “an increase” is meant a rise in the level of: (a) protein (e.g., as measured by ELISA or Western blot analysis); (b) reporter gene activity (e.g., as measured by reporter gene assay, for example, β-galactosidase, green fluorescent protein, or luciferase activity); (c) mRNA (e.g., as measured by RT-PCR or Northern blot analysis relative to an internal control, such as a “housekeeping” gene product, for example, β-actin or glyceraldehyde 3-phosphate dehydrogenase (GAPDH)); or (d) cell function, for example, as assayed by the number of apoptotic, mobile, growing, cell cycle arrested, invasive, differentiated, or dedifferentiated cells in a test sample. Desirably, the increase is by at least 1.5-fold to 2-fold, more desirably by at least 3-fold, and most desirably by at least 5-fold. As used herein, an increase may be the indirect result of PTGS, TGS, or another gene silencing event. For example, the dsRNA may inhibit the expression of a protein, such as a suppressor protein, that would otherwise inhibit the expression of another nucleic acid molecule.

By “long dsRNA” is meant a dsRNA that is at least 40, 50, 100, 200, 500, 1000, 2000, 50000, 10000, or more nucleotides in length. In some embodiments, the long dsRNA has a double stranded region of between 100 to 10000, 100 to 1000, 200 to 1000, or 200 to 500 contiguous nucleotides, inclusive. In some embodiments, the long dsRNA is a single strand which achieves a double-stranded structure by virtue of regions of self-complementarity (e.g., inverted repeats or tandem sense and antisense sequences) that result in the formation of a hairpin structure. In one embodiment, the long dsRNA molecule does not produce a functional protein or is not translated. For example, the long dsRNA may be designed not to interact with cellular factors involved in translation. Exemplary long dsRNA molecules lack a poly-adenylation sequence, a Kozak region necessary for protein translation, an initiating methionine codon, and/or a cap structure. In other embodiments, the dsRNA molecule has a cap structure, one or more introns, and/or a polyadenylation sequence. Other such long dsRNA molecules include RNA/DNA hybrids. Other dsRNA molecules that may be used in the methods of the invention and various means for their preparation and delivery are described in WO 00/63364, filed Apr. 19, 2000, the teaching of which is incorporated herein by reference.

By “modulates” is meant changing, either by a decrease or an increase. As used herein, desirably a nucleic acid molecule decreases the function of a cell, the expression of a target nucleic acid molecule in a cell, or the biological activity of a target polypeptide in a cell by least 20%, more desirably by at least 30%, 40%, 50%, 60% or 75%, and most desirably by at least 90%. Also as used herein, desirably a nucleic acid molecule increases the function of a cell, the expression of a target nucleic acid molecule in a cell, or the biological activity of a target polypeptide in a cell by at least 1.5-fold to 2-fold, more desirably by at least 3-fold, and most desirably by at least 5-fold.

By “multiple cloning site” is meant a known sequence within a DNA plasmid construct that contains a single specific restriction enzyme recognition site for one or more restriction enzymes, and that serves as the insertion site for a nucleic acid sequence. A multiple cloning site is also referred to as a polylinker or polycloning site. A wide variety of these sites are known in the art.

By “multiple epitope dsRNA” is meant an RNA molecule that has segments to derived from multiple target nucleic acids or that has non-contiguous segments from the same target nucleic acid. For example, the multiple epitope dsRNA may have segments derived from (i) sequences representing multiple genes of a single organism; (ii) sequences representing one or more genes from a variety of different organisms; and/or (iii) sequences representing different regions of a particular gene (e.g., one or more sequences from a promoter and one or more sequences from a coding region such as an exon). Desirably, each segment has substantial sequence identity to the corresponding region of a target nucleic acid. In various desirable embodiments, a segment with substantial sequence identity to the target nucleic acid is at least 30, 40, 50, 100, 200, 500, 750, or more nucleotides in length. In desirable embodiments, the multiple epitope dsRNA inhibits the expression of at least 2, 4, 6, 8, 10, 15, 20, or more target genes by at least 20, 40, 60, 80, 90, 95, or 100%. In some embodiments, the multiple epitope dsRNA has non-contiguous segments from the same target gene that may or may not be in the naturally occurring 5′ to 3′ order of the segments, and the dsRNA inhibits the expression of the nucleic acid by at least 50, 100, 200, 500, or 1000% more than a dsRNA with only one of the segments.

By “operably linked” is meant that a gene and one or more transcriptional regulatory sequences, e.g., a promoter or enhancer, are connected in such a way as to permit gene expression when the appropriate molecules (e.g., transcriptional activator proteins) are bound to the regulatory sequences.

By “partial RNA hairpin” is meant a hairpin that has a single stranded overhang, such as a 5′ or 3′ overhang. Desirably the partial hairpin will be encoded by a nucleic acid (e.g., a DNA molecule or vector). The encoded RNA molecule has, in 5′ to 3′ order, a first region of interest (Region 1), a loop region, and a second region of interest (Region 2). The regions of interest differ in length, and Region 1 has additional nucleotides at one end of the region that are not base-paired to nucleotides in the other region of interest (Region 2). One region of interest comprises a sequence of substantial identity to a target gene, and the other region of interest comprises a sequence of substantial complementarity to the target gene. In addition, the “partial” hairpin RNA may be a “forced” hairpin RNA (see FIG. 2B), in which case the Region 1, which includes either a sense or antisense sequence with respect to the target gene, will also include a Sequence A, and Region 2 will include a Sequence B, designed to base-pair with at least a portion of Sequence A, which serves to “force” the RNA to assume a hairpin structure. Optionally, Region 2 will include additional 3′ nucleotides complementary to nucleotides of Region 1. In a related aspect, the invention features an RNA molecule or a population of RNA molecules encoded by these nucleic acids. Exemplary RNA molecules are illustrated in FIG. 1A in which the first and second regions of interest are denoted “sense” and “antisense.” In each case, Region 1 may be either sense or antisense with respect to the target gene, and Region 2 will be the reverse complementary sequence, either antisense or sense with respect to the target gene, and capable of forming a hairpin structure. Desirably, the encoded RNA inhibits expression of the target gene in a cell or animal. Desirably, the partial RNA hairpin is extended in vitro or in vivo (e.g., in a cell or animal) with an RNA dependent-RNA polymerase). Desirably, extension of the partial hairpin produces a full hairpin.

By “phenotype” is meant, for example, any detectable or observable outward physical manifestation, such as molecules, macromolecules, structures, metabolism, energy utilization, tissues, organs, reflexes, and behaviors, as well as anything that is part of the detectable structure, function, or behavior of a cell, tissue, or living organism. Particularly useful in the methods of the invention are dsRNA mediated changes, wherein the detectable phenotype derives from modulation of the function of a cell, modulation of expression of a target nucleic acid, or modulation of the biological activity of a target polypeptide through dsRNA effects on a target nucleic acid molecule.

By “polypeptide biological activity” is meant the ability of a target polypeptide to modulate cell function. The level of polypeptide biological activity may be directly measured using standard assays known in the art. For example, the relative level of polypeptide biological activity may be assessed by measuring the level of the mRNA that encodes the target polypeptide (e.g., by reverse transcription-polymerase chain reaction (RT-PCR) amplification or Northern blot analysis); the level of target polypeptide (e.g., by ELISA or Western blot analysis); the activity of a reporter gene under the transcriptional regulation of a target polypeptide transcriptional regulatory region (e.g., by reporter gene assay, as described below); the specific interaction of a target polypeptide with another molecule, for example, a polypeptide that is activated by the target polypeptide or that inhibits the target polypeptide activity (e.g., by the two-hybrid assay); or the phosphorylation or glycosylation state of the target polypeptide. A compound, such as a dsRNA, that increases the level of the target polypeptide, mRNA encoding the target polypeptide, or reporter gene activity within a cell, a cell extract, or other experimental sample, is a compound that stimulates or increases the biological activity of a target polypeptide. A compound, such as a dsRNA, that decreases the level of the target polypeptide, mRNA encoding the target polypeptide, or reporter gene activity within a cell, a cell extract, or other experimental sample, is a compound that decreases the biological activity of a target polypeptide.

By “promoter” is meant a minimal sequence sufficient to direct transcription of a gene, including PolI, PolII, PolIII, mitochondrial, viral, bacterial, and other promoter sequences that are capable of driving transcription. Also included in this definition are those transcription control elements (e.g., enhancers) that are sufficient to render promoter-dependent gene expression controllable in a cell type-specific, tissue-specific, or temporal-specific manner, or that are inducible by external signals or agents; such elements, which are well-known to skilled artisans, may be found in a 5′ or 3′ region of a gene or within an intron. Desirably a promoter is operably linked to a nucleic acid sequence, for example, a cDNA or a gene in such a way as to permit expression of the nucleic acid sequence.

By “protein” or “polypeptide” or “polypeptide fragment” is meant any chain of more than two amino acids, regardless of post-translational modification (e.g., glycosylation or phosphorylation), constituting all or part of a naturally-occurring polypeptide or peptide, or constituting a non-naturally occurring polypeptide or peptide.

By “reporter gene” is meant any gene that encodes a product whose expression is detectable and/or able to be quantitated by immunological, chemical, biochemical, or biological assays. A reporter gene product may, for example, have one of the following attributes, without restriction: fluorescence (e.g., green fluorescent protein), enzymatic activity (e.g., β-galactosidase, luciferase, chloramphenicol acetyltransferase), toxicity (e.g., ricin A), or an ability to be specifically bound by an additional molecule (e.g., an unlabeled antibody, followed by a labelled secondary antibody, or biotin, or a detectably labelled antibody). It is understood that any engineered variants of reporter genes that are readily available to one skilled in the art, are also included, without restriction, in the foregoing definition.

By “selective conditions” is meant conditions under which a specific cell or group of cells can undergo selection. For example, the parameters of a fluorescence-activated cell sorter (FACS) can be modulated to identify a specific cell or group of cells. Cell panning, a technique known to those skilled in the art, is another method that employs selective conditions.

By “short dsRNA” is meant a dsRNA that has 45, 40, 35, 30, 27, 25, 23, 21, 18, 15, 13, or fewer contiguous nucleotides in length that are in a double stranded conformation. Desirably, the short dsRNA is at least 11 nucleotides in length. In desirable embodiments, the double stranded region is between 11 to 45, 11 to 40, 11 to 30, 11 to 20, 15 to 20, 15 to 18, 20 to 25, 21 to 23, 25 to 30, or 30 to 40 contiguous nucleotides in length, inclusive. In some embodiments, the short dsRNA is between 30 to 50, 50 to 100, 100 to 200, 200 to 300, 400 to 500, 500 to 700, 700 to 1000, 1000 to 2000, or 2000 to 5000 nucleotides in length, inclusive and has a double stranded region that is between 11 and 40 contiguous nucleotides in length, inclusive. In one embodiment, the short dsRNA is completely double stranded. In some embodiments, the short dsRNA is between 11 and 30 nucleotides in length, and the entire dsRNA is double stranded. In other embodiments, the short dsRNA has one or two single stranded regions. In particular embodiments, the short dsRNA binds PKR or another protein in a dsRNA-mediated stress response pathway. Desirably, the short dsRNA inhibits the dimerization and activation of PKR by at least 20, 40, 60, 80, 90, or 100%. In some desirable embodiments, the short dsRNA inhibits the binding of a long dsRNA to PKR or another component of a dsRNA-mediated stress response pathway by at least 20, 40, 60, 80, 90, or 100%.

By “specifically hybridizes” is meant a dsRNA that hybridizes to a target nucleic acid molecule but does not substantially hybridize to other nucleic acid molecules in a sample (e.g., a sample from a cell) that naturally includes the target nucleic acid molecule, when assayed under denaturing conditions. In one embodiment, the amount of a target nucleic acid molecule hybridized to, or associated with, the dsRNA, as measured using standard assays, is 2-fold, desirably 5-fold, more desirably 10-fold, and most desirably 50-fold greater than the amount of a control nucleic acid molecule hybridized to, or associated with, the dsRNA.

By “specifically inhibits the expression of a target nucleic acid molecule” is meant that inhibition of the expression of a target nucleic acid molecule in a cell or biological sample occurs to a greater extent than the inhibition of expression of a non-target nucleic acid molecule that has a sequence that is less than 99, 95, 90, 80, or 70% identical or complementary to that of the target nucleic acid molecule. Desirably, the inhibition of expression of the non-target molecule is 2-fold, desirably 5-fold, more desirably 10-fold, and most desirably 50-fold less than the inhibition of expression of the target nucleic acid molecule.

By “substantial sequence complementarity” is meant sufficient sequence complementarity between a dsRNA and a target nucleic acid molecule for the dsRNA to inhibit the expression of the nucleic acid molecule. Preferably, the sequence of the dsRNA is at least 40, 50, 60, 70, 80, 90, 95, or 100% complementary to the sequence of a region of the target nucleic acid molecule.

By “substantial sequence identity” is meant sufficient sequence identity between a dsRNA and a target nucleic acid molecule for the dsRNA to inhibit the expression of the nucleic acid molecule. Preferably, the sequence of the dsRNA is at least 40, 50, 60, 70, 80, 90, 95, or 100% identical to the sequence of a region of the target nucleic acid molecule.

By “target”, “target nucleic acid”, “target gene”, “target polynucleotide” or “target polynucleotide sequence” is meant any nucleic acid sequence present in a eukaryotic cell, plant or animal, vertebrate or invertebrate, mammalian, avian, etc., whether a naturally-occurring, and possibly defective, polynucleotide sequence, or a heterologous sequence present due to an intracellular or extracellular pathogenic infection or a disease, whose expression is modulated as a result of post-transcriptional gene silencing, transcriptional gene silencing, or other sequence-specific dsRNA-mediated inhibition. As used herein, the “target”, “target nucleic acid”, “target gene”, or “target polynucleotide sequence” may be in the cell in which the PTGS, transcriptional gene silencing (TGS), or other gene silencing event occurs, or it may be in a neighboring cell, or in a cell contacted with media or other extracellular fluid in which the cell that has undergone the PTGS, TGS, or other gene silencing event is contained. Such a “target”, “target nucleic acid”, “target gene”, or “target polynucleotide sequence” may be a coding sequence, that is, it is transcribed into an RNA, including an mRNA, whether or not it is translated to express a protein or a functional fragment thereof. Alternatively, it may be non-coding, but may have a regulatory function, including a promoter, enhancer, repressor, or any other regulatory element. The term “gene” is intended to include any target sequence intended to be “silenced”, whether or not transcribed and/or translated, including regulatory sequences, such as promoters.

Exemplary “target”, “target nucleic acid”, “target gene”, or “target polynucleotide sequence” molecules include nucleic acid molecules associated with cancer or abnormal cell growth, such as oncogenes, and nucleic acid molecules associated with an autosomal dominant or recessive disorder (see, for example, WO 00/63364, WO 00/44914, and WO 99/32619). Desirably, the dsRNA inhibits the expression of an allele of a nucleic acid molecule that has a mutation associated with a dominant disorder and does not substantially inhibit the other allele of the nucleic acid molecule (e.g., an allele without a mutation associated with the disorder). Other exemplary “target”, “target nucleic acid”, “target gene”, or “target polynucleotide sequence” molecules include host cellular nucleic acid molecules and pathogen nucleic acid molecules including coding and non-coding regions required for the infection or propagation of a pathogen, such as a virus, bacteria, yeast, protozoa, or parasite.

By “target polypeptide” is meant a polypeptide whose biological activity is modulated as a result of gene silencing. As used herein, the target polypeptide may be in the cell in which the PTGS, TGS, or other gene silencing event occurs, or it may be in a neighboring cell, or in a cell contacted with media or other extracellular fluid in which the cell that has undergone the PTGS, TGS, or other gene silencing event is contained.

By “transformation” or “transfection” is meant any method for introducing is foreign molecules into a cell (e.g., a bacterial, yeast, fungal, algal, plant, insect, or animal cell, particularly a vertebrate or mammalian cell). The cell may be in an animal. Lipofection, DEAE-dextran-mediated transfection, microinjection, protoplast fusion, calcium phosphate precipitation, viral or retroviral delivery, electroporation, and biolistic transformation are just a few of the transformation/transfection methods known to those skilled in the art. The RNA or RNA expression vector (DNA) may be naked RNA or DNA or local anesthetic complexed RNA or DNA (Pachuk et al., supra). Other standard transformation/transfection methods and other RNA and/or DNA delivery agents (e.g., a cationic lipid, liposome, or bupivacaine) are described in WO 00/63364, filed Apr. 19, 2000 (see, for example, pages 18-26). The dsRNAs or dsRNA expression constructs may also be complexed with the multifunctional molecular complexes of U.S. Pat. No. 5,837,533, U.S. Pat. No. 6,127,170, or U.S. Pat. No. 6,379,965 (Boutin), or the multifunctional molecular complexes or oil/water cationic amphiphile emulsions of PCT/US03/14288, filed May 6, 2003 (Satishchandran). Commercially available kits can also be used to deliver RNA or DNA to a cell. For example, the Transmessenger Kit from Qiagen, an RNA kit from Xeragon Inc., and an RNA kit from DNA Engine Inc. (Seattle, Wash.) can be used to introduce single or dsRNA into a cell.

By “transformed cell” or “transfected cell” is meant a cell (or a descendent of a cell) into which a nucleic acid molecule, for example, a dsRNA or double stranded expression vector has been introduced, by means of recombinant nucleic acid techniques. Such cells may be either stably or transiently transfected.

By “treating, stabilizing, or preventing cancer” is meant causing a reduction in the size of a tumor, slowing or preventing an increase in the size of a tumor, increasing the disease-free survival time between the disappearance of a tumor and its reappearance, preventing an initial or subsequent occurrence of a tumor, or reducing or stabilizing an adverse symptom associated with a tumor. In one embodiment, the percent of cancerous cells surviving the treatment is at least 20, 40, 60, 80, or 100% lower than the initial number of cancerous cells, as measured using any standard assay. Preferably, the decrease in the number of cancerous cells induced by administration of a composition of the invention is at least 2, 5, 10, 20, or 50-fold greater than the decrease in the number of non-cancerous cells. In yet another embodiment, the number of cancerous cells present after administration of a composition of the invention is at least 2, 5, 10, 20, or 50-fold lower than the number of cancerous cells present after administration of a vehicle control. Preferably, the methods of the present invention result in a decrease of 20, 40, 60, 80, or 100% in the size of a tumor as determined using standard methods. Preferably, at least 20, 40, 60, 80, 90, or 95% of the treated subjects have a complete remission in which all evidence of the cancer disappears. Preferably, the cancer does not reappear, or reappears after at least 5, 10, 15, or 20 years. In another desirable embodiment, the length of time a patient survives after being diagnosed with cancer and treated with a composition of the invention is at least 20, 40, 60, 80, 100, 200, or even 500% greater than (i) the average amount of time an untreated patient survives or (ii) the average amount of time a patient treated with another therapy survives.

By “treating, stabilizing, or preventing a disease or disorder” is meant preventing or delaying an initial or subsequent occurrence of a disease or disorder; increasing the disease-free survival time between the disappearance of a condition and its reoccurrence; stabilizing or reducing an adverse symptom associated with a condition; or inhibiting or stabilizing the progression of a condition. This includes prophylactic treatment, in which treatment before infection with an infectious agent, such as a virus, bacterium, or fungus, is established, prevents or reduces the severity or duration of infection. Preferably, at least 20, 40, 60, 80, 90, or 95% of the treated subjects have a complete remission in which all evidence of the disease disappears. In another embodiment, the length of time a patient survives after being diagnosed with a condition and treated using a method of the invention is at least 20, 40, 60, 80, 100, 200, or even 500% greater than (i) the average amount of time an untreated patient survives, or (ii) the average amount of time a patient treated with another therapy survives.

By “under conditions that inhibit or prevent an interferon response or a dsRNA stress response” is meant conditions that prevent or inhibit one or more interferon responses or cellular RNA stress responses involving cell toxicity, cell death, an anti-proliferative response, or a decreased ability of a dsRNA to carry out a PTGS or TGS event. These responses include, but are not limited to, interferon induction (both Type I and Type II), induction of one or more interferon stimulated genes, PKR activation, 2′5′-OAS activation, and any downstream cellular and/or organismal sequelae that result from the activation/induction of one or more of these responses. By “organismal sequelae” is meant any effect(s) in a whole animal, organ, or more locally (e.g., at a site of injection) caused by the stress response. Exemplary manifestations include elevated cytokine production, local inflammation, and necrosis. Desirably the conditions that inhibit these responses are such that not more than 95%, 90%, 80%, 75%, 60%, 40%, or 25%, and most desirably not more than 10% of the cells undergo cell toxicity, cell death, or a decreased ability to carry out a PTGS, TGS, or another gene silencing event, compared to a cell not exposed to such interferon response inhibiting conditions, all other conditions being equal (e.g., same cell type, same transformation with the same dsRNA expression library.

Apoptosis, interferon induction, 2′5′ OAS activation/induction, PKR induction/activation, anti-proliferative responses, and cytopathic effects are all indicators for the RNA stress response pathway. Exemplary assays that can be used to measure the induction of an RNA stress response as described herein include a TUNEL assay to detect apoptotic cells, ELISA assays to detect the induction of alpha, beta and gamma interferon, ribosomal RNA fragmentation analysis to detect activation of 2′5′OAS, measurement of phosphorylated eIF2a as an indicator of PKR (protein kinase RNA inducible) activation, proliferation assays to detect changes in cellular proliferation, and microscopic analysis of cells to identify cellular cytopathic effects. Desirably, the level of an interferon response or a dsRNA stress response in a cell transformed with a dsRNA or a dsRNA expression vector is less than 20, 10, 5, or 2-fold greater than the corresponding level in a mock-transfected control cell under the same conditions, as measured using one of the assays described herein. In other embodiments, the level of an interferon response or a dsRNA stress response in a cell transformed with a dsRNA or a dsRNA expression vector using the methods of the present invention is less than 500%, 200%, 100%, 50%, 25%, or 10% greater than the corresponding level in a corresponding transformed cell that is not exposed to such interferon response inhibiting conditions, all other conditions being equal. Desirably, the dsRNA does not induce a global inhibition of cellular transcription or translation.

By “viral infection” is meant the invasion of a host animal by a virus. For example, the infection may include the excessive growth of viruses that are normally present in or on the body of an animal or growth of viruses that are not normally present in or on the animal. More generally, a viral infection can be any situation in which the presence of a viral population(s) is damaging to a host animal. Thus, an animal is “suffering” from a viral infection when an excessive amount of a viral population is present in or on the animal's body, or when the presence of a viral population(s) is damaging the cells or other tissue of the animal.

Conditions and techniques that can be used to prevent an interferon response or dsRNA stress response during the methods of the present invention are described herein.

### BRIEF DESCRIPTION OF THE DRAWING

FIGS. 1A-1D are schematic illustrations of forced hairpin constructs. The sense (or antisense)-RNA sequence is followed by sequence A. Sequence A is immediately followed by the loop which is minimally at least about 7 nucleotides and maximally several hundred nucleotides. The loop is followed by sequence B which in turn is followed by the antisense (or sense) RNA sequence. Preferably, the loop does not base-pair with other sequences or form significant secondary structure within itself or with sequences A and B. For example, if sequence A consists of G's, then B consists of C's. In that case, the loop can consist of, e.g., only A's or only U's. A monosequence of U's in the loop is ordinarily a desirable embodiment, except in expression constructs having a U6 promoter, for which a string of 4-5 T's (in the DNA expression construct) serves as a terminator. Loop sequences such as CACACA . . . , ACACAC . . . , UUCUUC . . . , or CUUCUU . . . may also be used, as may other similar variations. Multiple G's are in general to be avoided in loops. Some degree of secondary structure within the loop is allowed if it is not significant. Sequences A and B are designed to immediately base-pair with each other, strongly driving the RNA sequence as a whole to assume a hairpin or stem-loop structure. In this sense, RNA structures containing the added self complementary A and B sequences flanking a loop sequence, are “forced” hairpins.

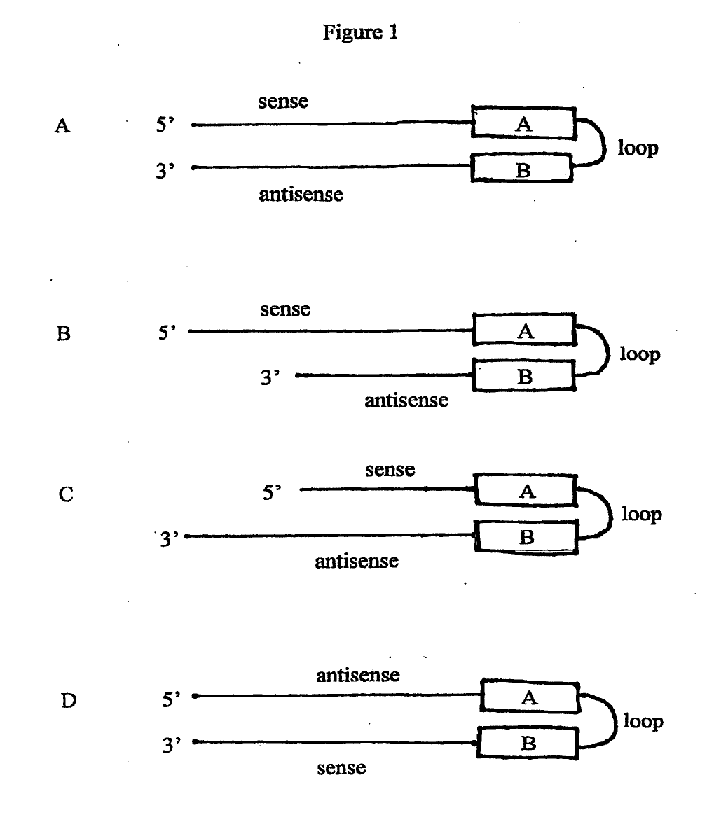

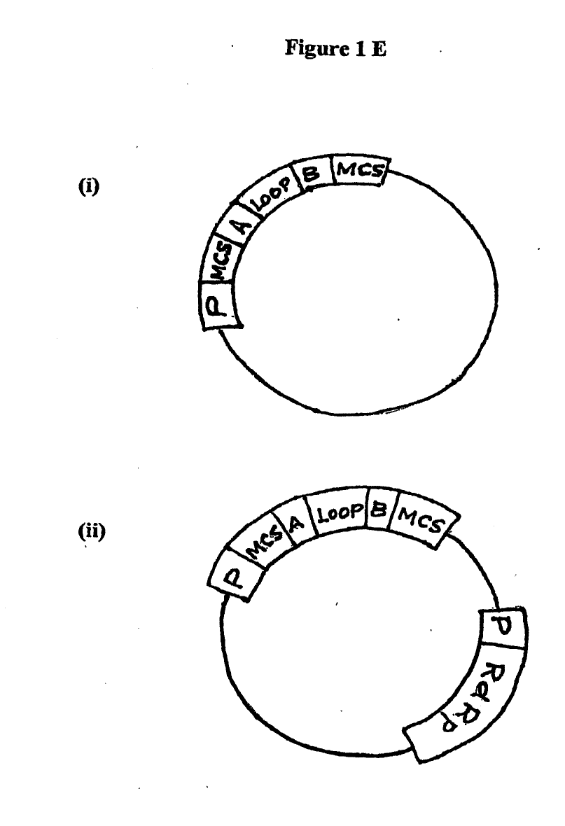

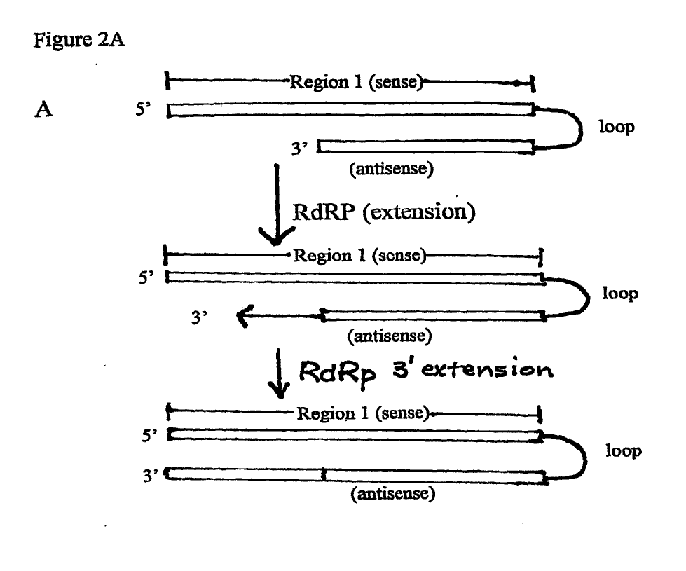

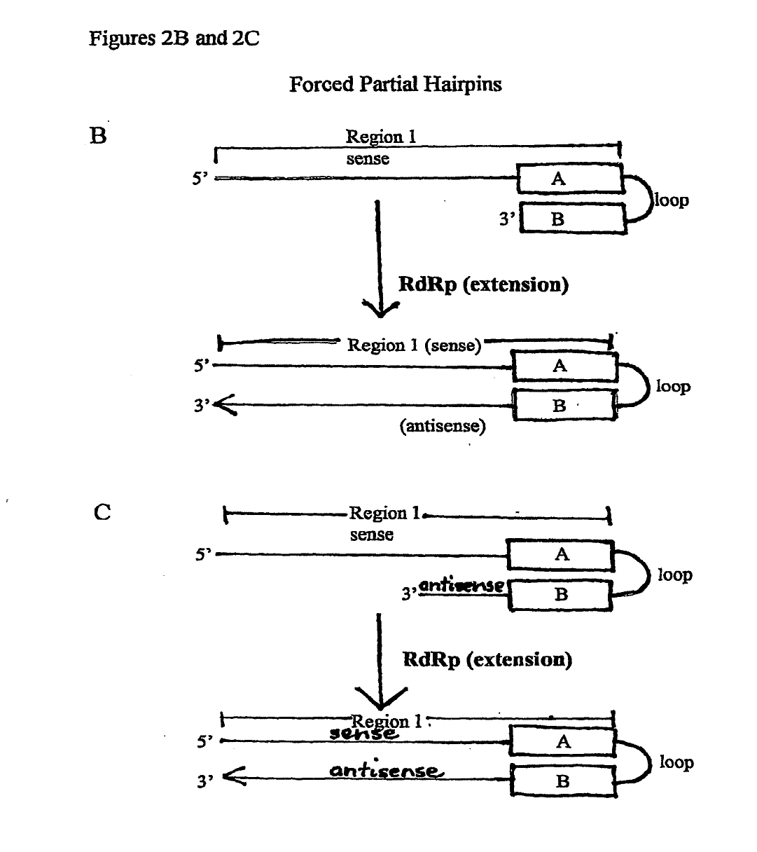

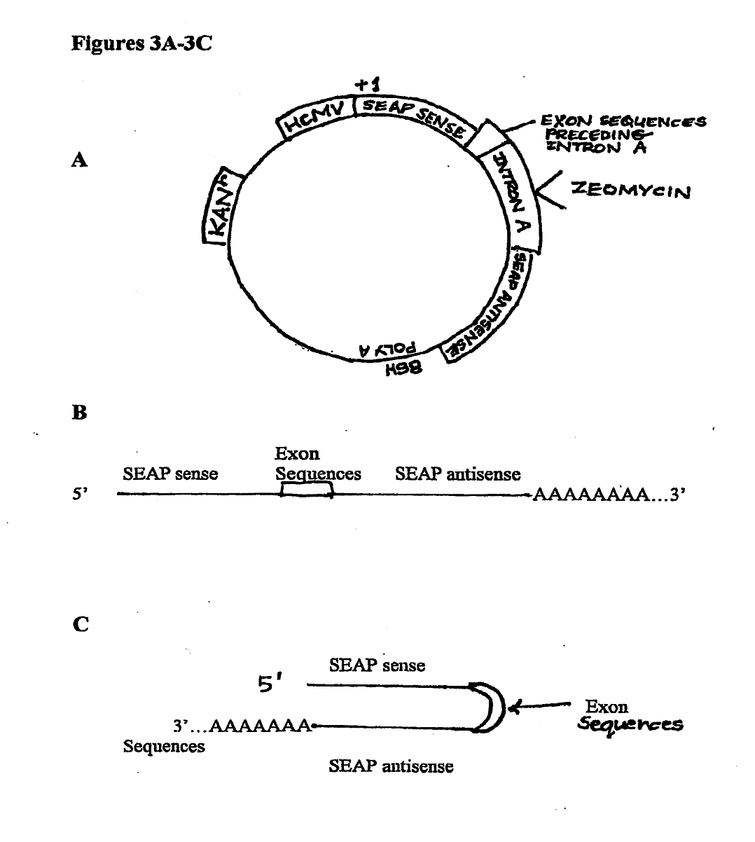

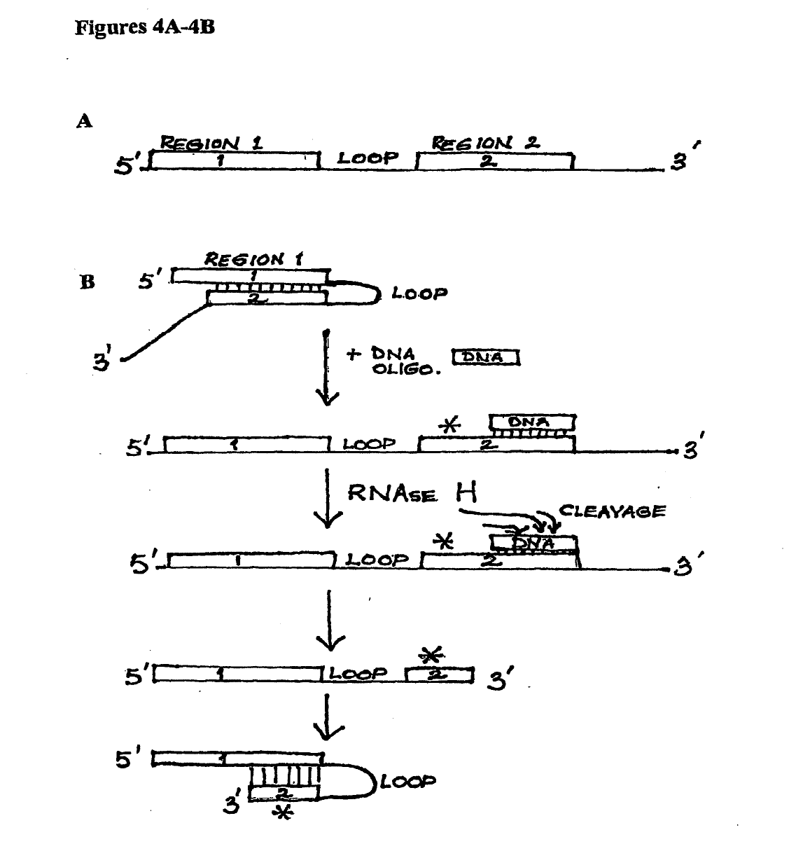

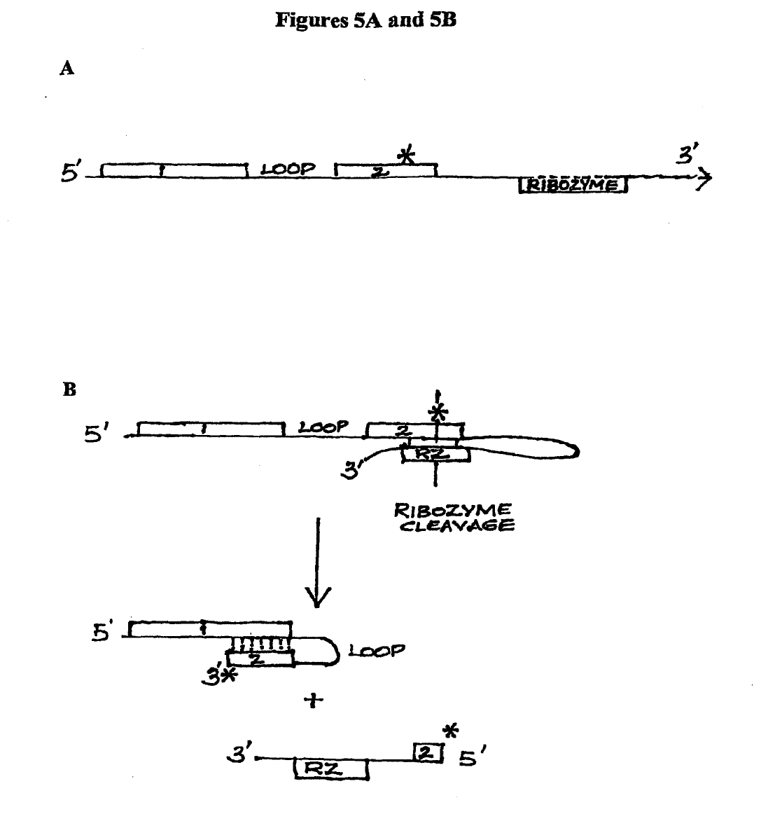

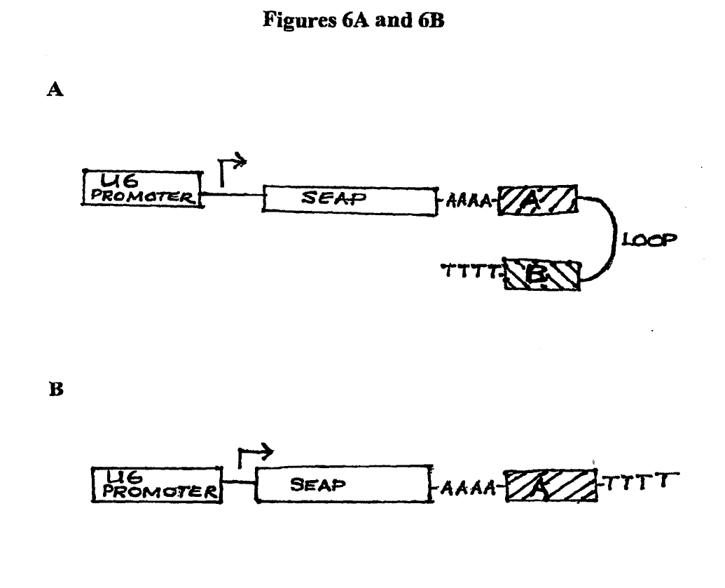

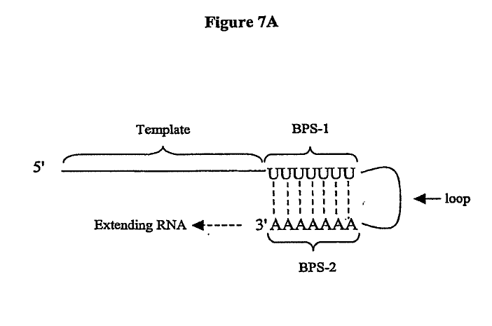

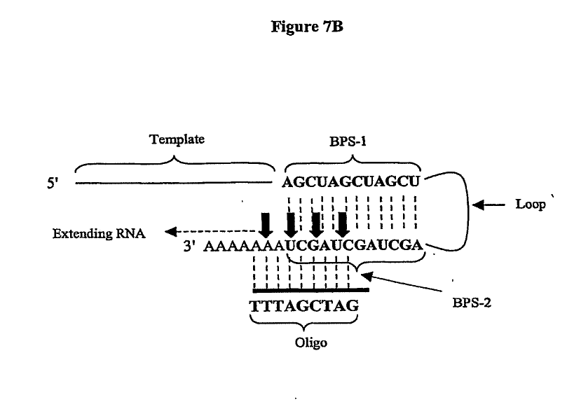

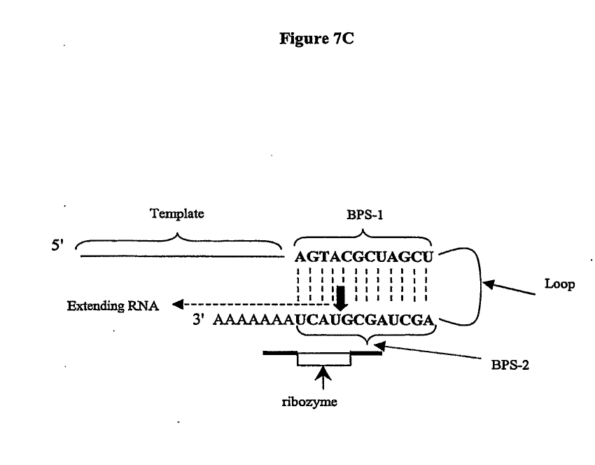

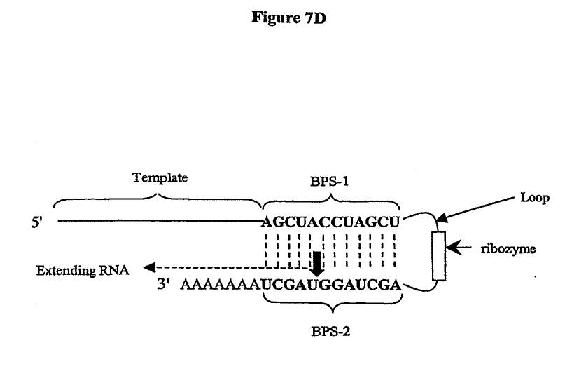

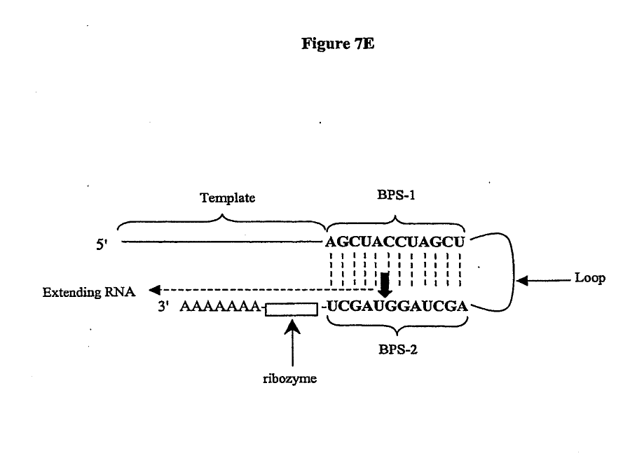

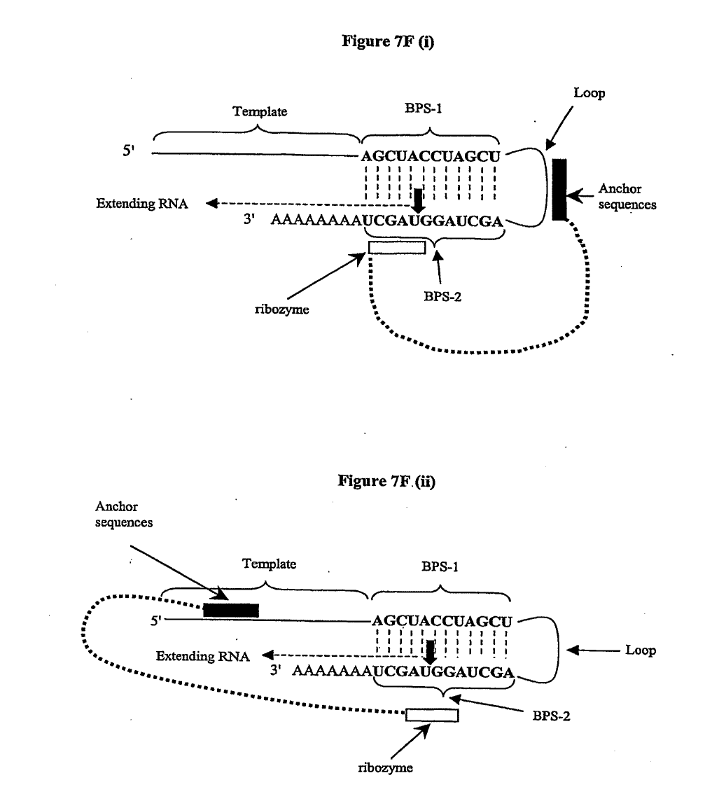

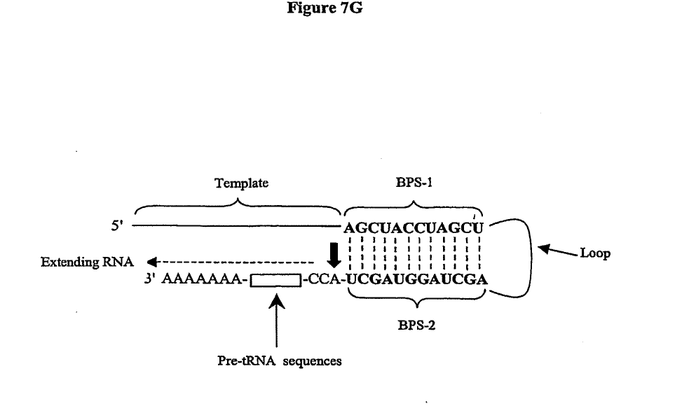

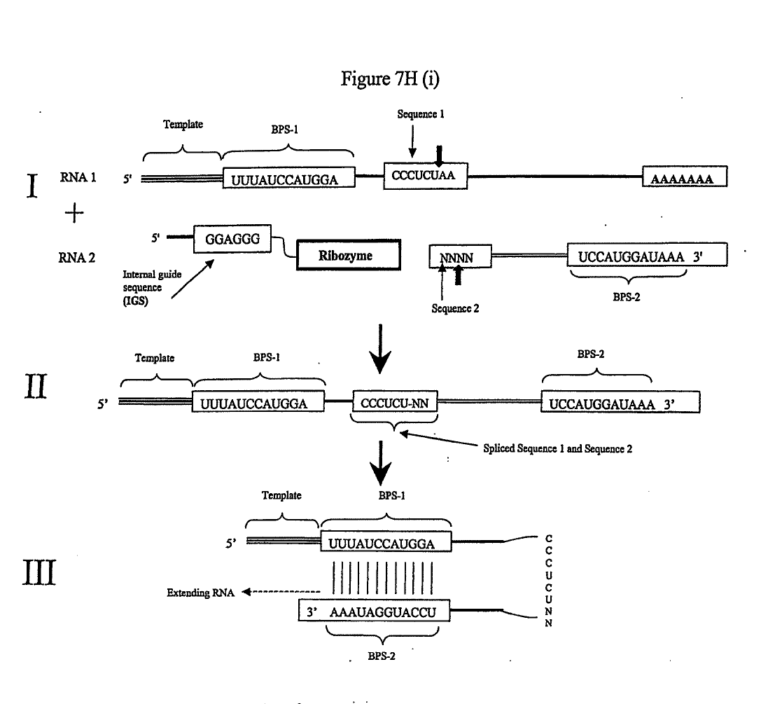

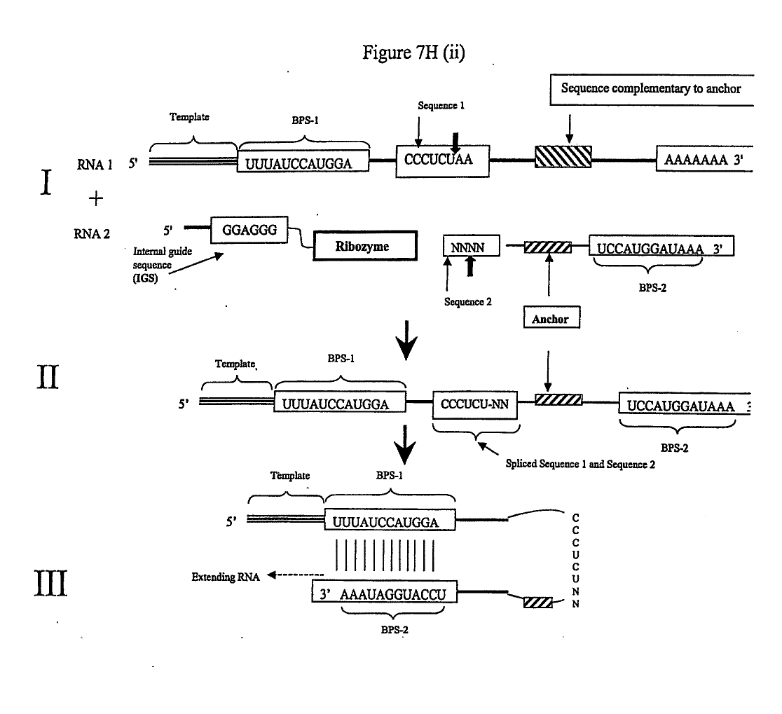

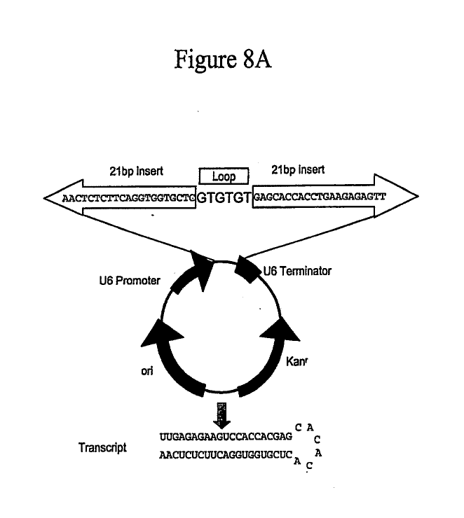

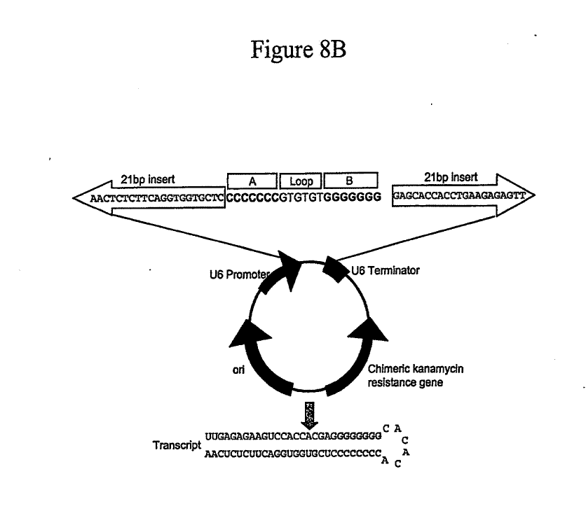

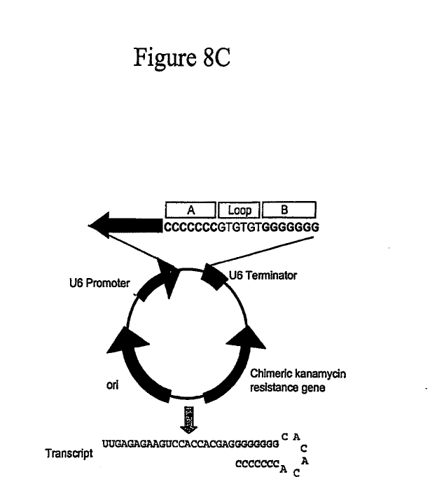

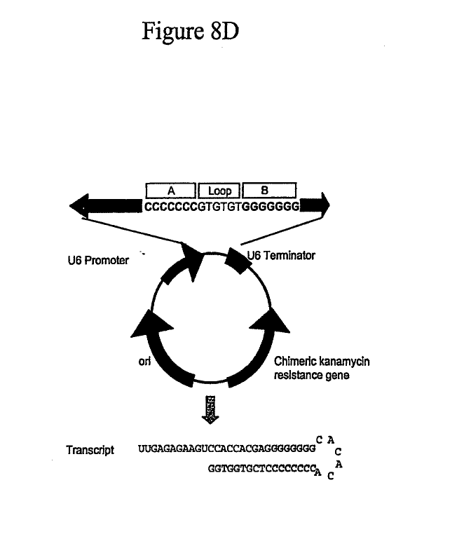

The antisense and sense RNA regions do not need to be the same length. For example, FIGS. 1B and 1C illustrate constructs in which the sense and antisense regions differ in length, resulting in an overhang region. FIG. 1D illustrates an alternative construct in which the 5′ end contains the antisense region and the 3′ end contains the sense region. In each case, the 5′ region may contain either the sense or antisense region with respect to a selected target (e.g., an mRNA) of interest, in which case the more 3′ region of the molecule may desirably contain an inverse complementary sequence (antisense or sense as the case may be), which may or may not be of the same length. In short hairpins, it may be preferred that the 5′ sequence be antisense and the 3′ sequence sense. Any of these constructs can also have additional sequences at the 5′ and/or 3′ end of the construct. A construct with a 5′ overhang, wherein the 3′ terminal nucleotides are base-paired to an upstream region and amenable to RdRp self-extension is a partial hairpin. Sequences A and B and the loop can be, e.g., sequences that are part of the cloning vector. Such a cloning vector may desirably be provided in a kit, optionally with a source of RdRp, for a variety of applications, including functional genomics.

FIGS. 1E(i) and 1E(ii) are schematic diagrams showing dsRNA expression constructs. The sense and antisense sequences can be cloned upstream and downstream of these sequence elements respectively, using standard methods (FIG. 1E). The sense insert can be cloned into one of the multiple cloning sites (MCS, which may be the same or different), and the antisense insert can be cloned into the other MCS. Any promoter known to one skilled in the art can be used to express this dsRNA. Such a standard cloning vector(s) with a promoter and two MCSs flanking A/Loop/B sequences (FIG. 1E(i)) can advantageously be provided in a commercial kit, e.g., useful for functional genomics applications, as described in more detail elsewhere herein. Bi- or multicistronic constructs comprising one or more of such promoter/MCS/A/Loop/B/MCS units, together with other expression units, e.g., expressing an RNA-dependent RNA polymerase (RdRp), (FIG. 1E(ii)), can be advantageously utilized, e.g., for expression and extension of “partial” hairpins as described herein.

FIGS. 2A, 2B, and 2C are schematic illustrations of partial hairpins that self-extend. In this example, the antisense sequence is self-extended using the sense RNA as a template (FIG. 2A). Alternatively, the antisense sequence can be near the 5′ end, and the sense sequence can be extended. A forced partial hairpin construct can also be used for self-extension (FIG. 2B). In this case, no sequence complementary to the sense or antisense strand is required because extension occurs directly from sequence B, or, if desired, from a short complementary antisense or sense sequence at the 3′ terminus (FIG. 2C).

FIG. 3A is a schematic illustration of a vector encoding a dsRNA with an intron. Because the HCMV immediate early promoter is recognized by RNA PolII, transcription initiates at ⁺1. The transcribed RNA includes the sense secreted alkaline phosphatase (SEAP) RNA followed by a human cytomegalovirus (HCMV) intron A containing a prokaryotic zeomycin expression cassette within the intron. The intron is followed by SEAP antisense RNA. The RNA is spliced co-transcriptionally and poly-adenylated, as directed by the BGH polyA site. A kanamycin resistance gene is also included.

FIG. 3B is an illustration depicting the structure of the linear RNA transcribed from the vector shown in FIG. 3A.

FIG. 3C is an illustration depicting the folding of the RNA of FIG. 3B into a hairpin with the exon sequences participating in the loop. This construct can also contain the features described for forced hairpin constructs and partial hairpins that self extend.

FIG. 4A is a schematic illustration of an RNA to be processed by RNase H-mediated cleavage. The linear representation from 5′ to 3′ of the precursor RNA includes a first region of interest (Region 1), a loop region, a second region of interest (Region 2), and a variable 3′ terminal sequence. As illustrated in FIG. 4B, Region 2 is the reverse complement of, and is capable of base-pairing with, a sequence of, in some aspects, a 3′ portion of Region 1 to form a hairpin structure with a free unpaired 3′ terminal sequence.

FIG. 4B is a schematic illustration of the folded structure of an RNA and the RNase H-mediated cleavage of the RNA. The 3′ terminal end of the molecule cannot be base-paired with the template. A DNA oligonucleotide is hybridized to a sequence in Region 2 such that there are a minimum of 5-15 base-pairs (*) in Region 2 upstream from the DNA/RNA hybridization sequences, and RNase H is added to cleave the DNA/RNA hybrid. The resulting partial hairpin can be self-extended. This RNase H cleavage approach represents one method of achieving a predetermined 3′ terminus which base-pairs with the template portion of the molecule, yielding a partial hairpin dsRNA that can be 3′ extended by an RdRp (RNA dependent-RNA polymerase).

FIG. 5A is a schematic illustration of an RNA to be processed by ribozyme-mediated cleavage. The linear representation from 5′ to 3′ of the precursor RNA includes Region 1, a loop sequence, Region 2 (which is the reverse complement of and capable of base-pairing with a 3′ portion of Region 1), an intervening sequence of from four to several hundred nucleotides (preferably 10 to 100, more preferably 10 to 50), and a hammerhead ribozyme designed to cleave a Target Ribozyme Cleavage Site (*) in Region 2. The cleavage site is designed so that there will be a minimum of 5-15 bases in Region 2 upstream from the cleavage site to maintain the RNA molecule in a double stranded conformation. The ribozyme is followed by a variable 3′ terminal sequence which does not base-pair with any upstream regions of the RNA molecule. If the RNA is not processed, it cannot be self-extended because the 3′ terminal sequence is not base-paired with the template.

FIG. 5B is a schematic illustration showing how ribozyme cleavage results in an RNA with a 3′ end that is base-paired with the template and thus can be self-extended to form a hairpin. In desirable embodiments, this ribozyme cleavage method can be utilized together with the forced hairpin methods. In that case, the RNA molecule will include additional Regions A and B flanking the loop region, and the Target Ribozyme Cleavage Site can be placed within Region B, as long as there are a minimum of 5-15 bases in Region B upstream from the Target Ribozyme Cleavage Site to base-pair with Region A and maintain the RNA molecule in a double-stranded conformation after cleavage.

FIG. 6A is a schematic illustration of a system that facilitates the single step cloning and dsRNA expression from mammalian plasmid vectors. In particular, FIG. 6A depicts RNA polIII transcription (U6 promoter; angled arrow) through the cDNA (SEAP) and the terminal vector derived hairpin (i.e., “A” and “B” boxes with diagonal stripes, which include self-complementary sequences that flank the loop sequence) which finally terminates at a string of T's. There are a minimum of 4-5 T residues in the vector and therefore, a corresponding number of A residues in the complementary sequence. Advantageously, the vector-derived hairpin can be the same as the forced hairpin constructs described herein (e.g., reverse complementary sequences “A” and “B” flanking the loop) except for the T residues located just downstream of the “B” box and the A residues located just upstream of the “A” box. Such a vector generates a transcript with a 3′ terminus that serves as a perfect primer in cis for the second strand synthesis (extension) by the mammalian RdRp creating the desired hairpin dsRNA to serve as an initiator of dsRNA-mediated gene silencing. This “terminator” approach represents one method of designing a dsRNA expression construct which expresses an RNA with a predetermined 3′ terminus which base-pairs with the template portion of the molecule, yielding a partial hairpin dsRNA that can be 3′ extended by a RdRp (RNA dependent-RNA polymerase).

FIG. 6B depicts the control plasmid vectors that lack the terminal hairpin but are still capable of expressing and terminating a single strand of the target cDNA.

FIGS. 7A-7H are schematic illustrations of methods for generating a partial hairpin that can be extended by an RNA dependent-RNA polymerase. RNA PolII transcripts are shown but the transcript may be derived from other RNA polymerases, including both RNA and DNA dependent RNA polymerases. The dsRNA hairpin molecules depicted in FIGS. 7A-7H, consist of, in 5′ to 3′ order, a “template” portion of the molecule, which is either identical to (sense), or complementary to (antisense), an mRNA that is the target for dsRNA-mediated gene silencing, a base-paired sequence 1 (BPS-1), a loop region, and a base-paired sequence 2 (BPS-2). The sequences labeled as BPS-1 and BPS-2 are selected to schematically show base-pairing, but may be derived from sequences in the target mRNA molecule, or they may be synthetic sequences engineered to force specific base-pairing to promote efficient formation of a hairpin molecule.

FIG. 7A is a schematic that depicts a dsRNA hairpin with a polyU tract that was generated by polIII transcription of a polyT sequence that was incorporated into an expression vector. The polyU tract is downstream from the 5′ template region of the dsRNA hairpin. This approach enables a subset of transcript molecules to assume a hairpin conformation in which the polyU tract base-pairs with the polyA sequence incorporated into the transcript during transcription. If non-polyadenylating polymerases are used, such as T7, a polyA sequence could also be incorporated into the vector downstream of the polyT sequence to produce transcripts that have a polyU tract, as well as a polyA sequence at the 3′ end. If non-polyadenylating polymerases are used, such as PolIII, which terminates at a stretch of 4 T's, the polyU and polyA tracts would be replaced by sequences that do not have a stretch of 4 T's. In some embodiments, RNA polII transcripts that have been polyadenylated are used since a stretch of polyU in the template strand allows a fraction of the mRNA molecules to assume a hairpin structure in which the terminal A sequences are flush base-paired with the template polyU.

FIG. 7B is a schematic that shows cleavage of the 3′ end of a dsRNA hairpin at one or more sites (designated by [symbol] ) within “BPS-2” using RNase H to generate a “partial” dsRNA hairpin. Cleavage by RNase H is mediated by the addition of an oligodeoxyribonucleotide (designated as “oligo”) that is complementary to, and that base-pairs with, BPS-2 at the 3′ end of the dsRNA hairpin, thereby forming a DNA/RNA hybrid that is recognized and cleaved by RNase H. Cleavage of the dsRNA hairpin with RNase H generates a “partial” dsRNA hairpin with a 3′ end that remains base-paired to “BPS-1” of the same molecule. The sequence at the extreme 5′ end of the dsRNA hairpin (designated as “template”) is either identical to (sense), or complementary to (antisense), an mRNA that is the target for dsRNA-mediated gene silencing. This sequence serves as the “template” for RdRp extension of the 3′ end of the “partial” dsRNA hairpin, if desired, into a full hairpin.

In addition to the use of an oligodeoxyribonucleotide, a “partial” dsRNA hairpin can also be generated by providing a modified oligonucleotide that base-pairs with BSP-2 and allows RNAse H cleavage of the RNA sequence. Such a modified oligonucleotide will be modified at the 2′ position of the ribose sugar by addition of a modifier group. See, e.g., the teaching of WO95/17414A1, incorporated herein by reference. The sequences flanking the loop (BPS-1 and BPS-2) were selected to schematically show base-pairing, but could be derived from sequences in the target mRNA molecule or synthetic sequences engineered to force specific base-pairing in order to drive efficient hairpin formation.

FIGS. 7C-7F are schematics that demonstrate a ribozyme cleavage approach for generating a predetermined 3′ terminus that base-pairs with the upstream “template” portion of the molecule, which is either identical to (sense), or complementary to (antisense), an mRNA that is the target for dsRNA-mediated gene silencing. As the schematic shows, this approach yields a partial dsRNA hairpin that can be 3′ extended by an RdRp (RNA dependent-RNA polymerase). As shown, the ribozyme can be supplied to work in trans (FIG. 7C), or can be placed within the RNA molecule itself, e.g., within the loop (FIG. 7D) or within the 3′ terminal sequences of the RNA transcript (FIG. 7E). In FIGS. 7C, 7D, 7E, and 7F (i) and 7F (ii), BPS-1 (AGCUACCUAGCU) and BPS-2 (UCGAUGGAUCGA), which flank the loop, are selected as examples to schematically show base-pairing. These sequences may be derived from sequences in the target mRNA molecule, or they may be synthetic sequences engineered to force specific base-pairing to promote efficient formation of a hairpin molecule.

FIG. 7C is a schematic that depicts cleavage of the dsRNA hairpin (designated by [symbol] ) by a ribozyme provided in trans, e.g., by exogenous delivery of the ribozyme, or by expression of the ribozyme from a separate plasmid or separate cistron.

FIG. 7D is a schematic that depicts cleavage of the 3′ sequence of the dsRNA hairpin (designated by [symbol] ) by a ribozyme placed within the hairpin loop.

FIG. 7E is a schematic that depicts cleavage of the dsRNA hairpin within BPS-2 (designated by [symbol] ) by a ribozyme placed in the 3′ portion of the RNA template. An RNA polII transcript is shown, but the transcript can be derived from other RNA polymerases, e.g., RNA and DNA dependent RNA polymerases.

FIGS. 7F(i) and 7F(ii) are schematics that depict cleavage of the dsRNA hairpin, within BPS-2 (designated by [symbol] ), by an anchored ribozyme, as described in U.S. Pat. No. 6,080,851, “Ribozymes with linked anchor sequences”, Pachuk et al., the teaching of which is incorporated herein by reference. The rate and the specificity of the ribozyme cleavage can be further enhanced, if desired, through the use of an anchor sequence that base-pairs with a sequence in the transcript, preferably a portion of the hairpin loop sequence. Such anchored ribozymes can be delivered exogenously into the cell or organism, or can be co-expressed with the RNA to be cleaved, e.g., on a separate plasmid or a separate cistron of a bi- or multi-cistronic plasmid.

FIG. 7G is a schematic that depicts the use of a pre-tRNA sequence placed at the 3′ end of the transcript which promotes RNase P cleavage of the dsRNA hairpin (designated by [symbol] ) in trans.

FIGS. 7H(i) and 7H(ii) are schematics depicting the utilization of trans-splicing ribozymes to generate hairpins from two separate RNA molecules. The sequences selected in FIGS. 7H (i) and (ii) were selected to schematically show base-pairing, but could be derived from sequences in the target mRNA molecule or synthetic sequences engineered to force specific base-pairing in order to drive efficient hairpin formation. FIG. 7H(i) depicts direct trans-splicing and FIG. 7H(ii) depicts anchored trans-splicing. See, e.g., the trans-splicing ribozyme structures of U.S. Pat. No. 5,667,969 and U.S. Pat. No. 5,874,414, the teaching of which is incorporated herein by reference.

FIG. 7H (i) demonstrates three stages in this trans-splicing approach. Stage I depicts the base-pairing and cleavage of two RNA molecules (designated “RNA 1” and “RNA 2”). RNA 1 contains a sequence at the extreme 5′ end (designated as “template”) that is either identical to (sense), or complementary to (antisense), an mRNA that is the target for dsRNA-mediated gene silencing. RNA 1 also contains “BPS-1,” which is selected to base-pair with the complementary sequence “BPS-2” which, in this case, is present in the second RNA molecule, RNA 2. Finally, RNA 1 contains “sequence 1,” which base pairs with the internal guide sequence (IGS) present in RNA 2. RNA 2 is an RNA molecule that lacks a polyA tail (e.g., it is generated by polIII, it is derived from other polymerases, or it is synthetically prepared and co-administered with RNA 1). The base-pairing of sequence 1 in RNA 1 with the IGS in RNA 2 enables the “ribozyme,” also present in RNA 2, to cleave RNA 1 and RNA 2 in trans at the indicated sites (designated by [symbol] ) and to re-ligate RNA 1 (at sequence 1) and RNA 2 (at sequence 2) to form a single chimeric RNA molecule.

Stage II demonstrates the newly formed chimeric RNA molecule containing a region formed by the re-ligation of sequence 1 of RNA 1 and sequence 2 of RNA 2.

Stage III demonstrates the formation of a “partial” dsRNA hairpin due to the base-pairing of BPS-1 and BPS-2. The upstream 5′ end of the molecule (“template”) serves as the template for extension of the dsRNA hairpin by a RdRp.

FIG. 7H (ii) demonstrates the same three stages of the trans-splicing approach presented in FIG. 7H (i), except that an anchor sequence is present in RNA 2. The complement to the anchor sequence is present in RNA 1, as indicated. The anchor sequence can be placed almost anywhere in the RNA molecule, although, the placement of the anchor sequence should not compromise ribozyme function. The anchor sequence can be placed in the RNA molecule so that it is not included in the final dsRNA hairpin molecule.

FIGS. 8A-8D are diagrams showing expression vectors containing nucleotide sequence for generating an RNA hairpin. The 21 base-pair sequence provided in FIGS. 8A-8D corresponds to nucleotides 2912-2932 of the hepatitis B virus (HBV; strain ayw) genome sequence found in Genbank Accession Nos.: V01460 and J02203. This sequence targets multiple transcripts in the HBV genome including the surface antigens, polymerase, and core transcripts. The 5′ arm of the hairpin (UUGAGAGAAGUCCACCACGAG) is the antisense strand for all the transcripts shown.

FIG. 8A shows an expression vector that encodes a complete expressed hairpin. The expressed hairpin is exemplified by a 21 base-pair HBV sequence, and its complement, which flank a short GTGTGT loop sequence. The sequence is transcribed under the control of the U6 promoter and terminator sequences, which produce the indicated transcript. Once transcribed, the transcript forms a hairpin structure due to base-pairing between the 21 base-pair HBV sequence and its complement. For small hairpin structures, as shown, the 5′ end of the transcript preferably corresponds to the antisense sequence of the target mRNA.

FIG. 8B shows an expression vector that encodes a transcript having a 21 base-pair HBV sequence, and its complement, which flank, an A sequence, a loop region, and a B sequence. The A and B sequences are designed to immediately base-pair with each other, strongly driving the RNA sequence as a whole to assume a hairpin or stem-loop structure. The loop region is designed to avoid any base-pairing or the formation of secondary structure. The A and B sequences in the plasmid construct can code for a transcript with one type of nucleotide strand (i.e., polyC or polyG), as shown, or they can code for a string of “GC” repeats. Once transcribed, the transcript forms a forced hairpin structure due to base-pairing between the A and B sequences, followed by base-pairing between the 21 base-pair HBV sequence and its complement.

FIG. 8C shows an expression construct for expression of a forced partial RNA hairpin. This construct provides A and B sequences that flank a short loop region. The A and B sequences are designed to immediately base-pair with each other, strongly driving the RNA sequence as a whole to assume a hairpin or stem-loop structure. The loop region is designed to avoid any base-pairing or the formation of secondary structure. The long arrow (represented by an “ [symbol] ”) that precedes the A sequence represents the 21 base-pair HBV sequence. Once transcribed, the transcript forms a forced hairpin structure due to base-pairing between the A and B sequences. The forced hairpin is referred to as “partial” because the resulting RNA transcript has a 21 base-pair overhang, corresponding to the 21 base-pair HBV sequence, at the 5′ end of the transcript. Extension of the 3′ end to form a full hairpin can be accomplished by using an RdRp.

FIG. 8D shows an expression construct for expression of a forced partial RNA hairpin. This construct provides A and B sequences that flank a short loop region. The A and B sequences are designed to immediately base-pair with each other, strongly driving the RNA sequence as a whole to assume a hairpin or stem-loop structure. The loop region is designed to avoid any base-pairing or the formation of secondary structure. The long arrow (represented by an “ [symbol] ”) that precedes the A sequence represents the 21 base-pair HBV sequence. The shorter arrow (represented by an “ [symbol] ”) that follows the B sequence represents a shorter 10 base-pair region of the 21 base-pair HBV sequence. Once transcribed, the transcript forms a forced hairpin structure due to base-pairing between the A and B sequences. The forced hairpin is again referred to as “partial” because only 10 nucleotides of the HBV sequence overlap and base-pair, leaving an 11 nucleotide overhang at the 5′ end of the transcript. Extension of the 3′ end to form a full hairpin can be accomplished by using an RdRp.

### DETAILED DESCRIPTION

We have previously reported that induction of the undesired interferon response and activation of the various components comprising this response is mediated by the particular dsRNA delivery/expression method used. Importantly, not all methods of dsRNA presentation activate this response (see, e.g., U.S. Ser. No. 60/378,191, filed May 6, 2002; 60/375,636; filed Apr. 26, 2002; 10/062,707, filed Jan. 31, 2002; U.S. Published Application 2002/0132257 and European Published Application EP 1229134 which are each hereby incorporated by reference).

The forementioned applications describe a schematic illustration of the RNA stress response pathway, also known as the Type I interferon response (see, e.g., FIG. 2 of U.S. Ser. No. 60/375,636; and FIG. 4 of U.S. Ser. No. 10/062,707; U.S. Published Application 2002/0132257; and EP1229134). The pathway is branched and RNA mediated induction/activation can occur at multiple points in the pathway. RNA (dsRNA and other structures) can act to elicit the production of alpha and/or beta interferon in most cell types. Early and key events in the interferon response pathway include interferon-mediated activation of the Jak-Stat pathway, which involves tyrosine-phosphorylation of STAT proteins (STATs). Activated STATs translocate to the nucleus and bind to specific sites in the promoters of IFN-inducible genes thereby effecting transcription of these genes, the expression of which acts in concert to push the cell towards apoptosis or to an anti-proliferative state. There are hundreds of interferon-stimulated genes but only two of the better characterized ones, PKR and 2′5′-OAS, have been shown. RNA can also activate the pathway in an interferon- and STAT-independent manner. In addition, dsRNA/structured RNA can also activate inactive PKR and 2′5′-OAS which are constitutively expressed in many cell types.

Activation of this undesired RNA stress response may require a specific dsRNA sub-cellular localization, higher order structure, and/or amount of cellular dsRNA. For example, we have developed an in vivo expression system for dsRNA (e.g., long dsRNA over 100 base-pairs, desirably over 200 base-pairs, and more desirably over 600 base-pairs) that efficiently induces PTGS without inducing/activating the RNA stress response pathway. Using this system, we have demonstrated the long-term suppression of prostate specific antigen (PSA) and secreted human placental alkaline phosphatase in a human cell line.

The present invention features a variety of novel methods and nucleic acids for silencing genes that produce few, if any, toxic side-effects. In particular, these methods involve administering to a cell or animal an agent that provides one or more double stranded RNA (dsRNA) molecules that have substantial sequence identity to a region of a target nucleic acid and that specifically inhibit the expression of the target nucleic acid. In some embodiments, a portion or all of the dsRNA molecules are located in the cytoplasm and thus mediate post-transcriptional gene silencing (PTGS). In certain embodiments, a portion or all of the dsRNA molecules are located in the nucleus and mediate transcriptional gene silencing (TGS). For TGS applications, the dsRNA desirably includes a regulatory sequence (e.g., a transcription factor binding site or a promoter) and/or a coding sequence, and for PTGS applications, the dsRNA desirably includes a regulatory sequence (e.g., a 5′ or 3′ untranslated region (UTR) of an mRNA) and/or a coding sequence. For methods in which the dsRNA is made in the nucleus and PTGS is desirable, the dsRNA may optionally include one or more constitutive transport element (CTE) sequences or introns to promote transport of the dsRNA into the cytoplasm and/or include a polyA tail to promote dsRNA stability. Desirably, the same dsRNA mediates both TGS and PTGS. In other embodiments, one or more dsRNA molecules that mediate TGS and one or more dsRNA molecules that mediate PTGS are used.

A variety of methods have been developed to inhibit or prevent an interferon or RNA stress response. One such method is based on the surprising discovery that short dsRNA molecules (e.g., dsRNA molecules containing a region of between 11 and 40 nucleotides in length that is in a double stranded conformation) can be used to inhibit the PKR/interferon/stress/cytotoxicity response induced by other dsRNA molecules (e.g., short or long dsRNA molecules homologous to one or more target genes) in vertebrate cells, tissues, and organisms (See U.S. Ser. No. 60/375,636; filed Apr. 26, 2002 and U.S. Ser. No. 10/425,006 filed Apr. 28, 2003, “Methods of Silencing Genes Without Inducing Toxicity”, C. Pachuk, both of which are incorporated herein by reference.). In particular, two short dsRNA molecules prevented the toxic effects that are normally induced by the dsRNA poly(I)(C). Thus, these methods inhibit the induction of non-specific cytotoxicity and cell death by dsRNA molecules (e.g., exogenously introduced long dsRNA molecules) that would otherwise preclude their use for gene silencing in vertebrate cells and vertebrates.

Other approaches for dsRNA-mediated gene silencing without induction of the interferon response involve intracellular expression, either in the cytoplasm or the nucleus, of dsRNA (e.g., a long dsRNA) with substantial identity to a target gene. Surprisingly, this method allows for the sustained expression of long dsRNA within cells without invoking the components of the dsRNA stress or type I interferon response pathway. In particular, gene silencing was observed using nuclear expression of dsRNA from RNA polII, RNA polIII, and T7 constructs, and using cytoplasmic expression of dsRNA. Thus, generation of dsRNA in vivo is an efficient and practicable method for inducing long-term gene silencing in mammalian and other vertebrate systems. Furthermore, intracellular expression of long dsRNA was a very potent inducer of gene silencing. For example, long dsRNA was able to down-regulate the expression of target genes by 95% for at least one month. Additionally, long dsRNA may be more effective for some applications than short RNA in the degree and/or the duration of gene silencing. Long-term maintenance of the silencing response is important in many silencing applications such as functional genomics and target validation because many cell models for studying gene function and validating gene targets require sustained loss of targeted gene function. Long-term gene silencing is also desirable for many therapeutic purposes.

If desired, expression of a target gene can be further inhibited by RNA replication of dsRNA with substantial identity to the target gene. For example, an RNA dependent-RNA polymerase can be expressed in a cell or animal into which the dsRNA or a vector encoding the dsRNA is introduced. The RNA dependent-RNA polymerase amplifies the dsRNA and desirably increases the number of dsRNA molecules in the cell or animal by at least 2, 5, or 10-fold. The RNA dependent-RNA polymerase is naturally expressed by the cell or animal, is encoded by the same vector that encodes the dsRNA, or is encoded by a different vector. Exemplary RNA dependent-RNA polymerases include viral, plant, invertebrate, or vertebrate (e.g., mammalian or human) RNA dependent-RNA polymerases. In other approaches, long-term gene silencing is enhanced by expressing the dsRNA from a vector that has an origin of replication that permits replication of the vector in the cell or animal. Desirably, the vector is maintained in the progeny of the cell or animal after 10, 30, 50, 100, or more cell divisions or after one week, one month, six months, or one year.

Additionally, gene silencing can be enhanced by using partial or full RNA hairpins to silence a target gene. In particular, dsRNA may be generated more efficiently from a single-stranded RNA with inverted repeat sequences that forms a dsRNA hairpin structure than from two separate RNA molecules that must hybridize in vitro or in vivo to form dsRNA. In various embodiments, the dsRNA is a partial RNA hairpin that has a single stranded overhang or a full RNA hairpin without a single stranded overhang. In the hairpins, one region has substantial identity to a target gene and is base-paired to another region of interest that has substantial complementarity to the target gene. If desired, the dsRNA can include additional base-paired regions to increase the efficiency of hairpin formation; for example, the dsRNA can include a loop that is flanked by a base-paired helix which promotes hairpin formation.

The invention also provides novel methods for generating hairpins in vitro or in vivo. These methods involve producing a partial hairpin that has a single stranded overhang and extending the partial hairpin so that the single stranded overhang decreases in size. In particular, the partial hairpin has a 3′ end that is base-paired with another region in the partial hairpin, and the 3′ end of the partial hairpin is extended by an RNA dependent-RNA polymerase (e.g., a viral, plant, invertebrate, or vertebrate RNA dependent-RNA polymerase, such as mammalian or human RNA dependent-RNA polymerase).

The above dsRNA molecules and vectors can be used in a variety of methods for treating, stabilizing, or preventing a disease or disorder in an animal (e.g., an invertebrate or a vertebrate, such as a mammal or human). In these methods, a dsRNA or a vector encoding a dsRNA that has substantial sequence identity to a region of a target nucleic acid associated with the disease or disorder, and that specifically inhibits the expression of the target nucleic acid, is administered to the animal. In some embodiments, the target gene is a gene associated with cancer, such as an oncogene, or a gene encoding a protein associated with a disease, such as a mutant protein, a dominant negative protein, or an overexpressed protein. Moreover, the dsRNA molecules can be used to treat, stabilize, or prevent an infection by a pathogen such as a virus, bacteria, or yeast. In some embodiments, the target nucleic acid is a gene of the pathogen that is necessary for replication and/or pathogenesis, or a gene encoding a cellular receptor necessary for a cell to be infected with the pathogen.

The invention also features the use of the above dsRNA molecules and dsRNA expression vectors in methods which utilize dsRNA-mediated gene silencing for functional genomics applications, including high throughput methods of using dsRNA-mediated gene silencing to identify a nucleic acid molecule that modulates a detectable phenotype of a cell, e.g., a function of the cell, expression of a target nucleic acid molecule, or biological activity of a target polypeptide. These methods involve transfection of libraries of dsRNA molecules or libraries of vectors encoding dsRNA molecules into cells to inhibit gene expression. The inhibition of gene expression modulates a detectable phenotype of a cell and allows the nucleic acid molecule responsible for the modulation to be readily identified.

### EXAMPLES

The following examples are to illustrate the invention. They are not meant to limit the invention in any way. For example, it is noted that any of the following examples can be used with dsRNA molecules of any length and structure, including any of the dsRNA structures of the invention, which include a double-stranded region, one strand of which is substantially identical to a region of a target nucleic acid. The methods of the present invention can be readily adapted by one skilled in the art to utilize multiple dsRNA molecules and/or multiple dsRNA expression constructs to inhibit multiple target nucleic acid molecules. Any of the dsRNA molecules, target nucleic acid molecules, or methods described in, e.g., in U.S. Published Application 2002/0132257 and European Published Application EP 1229134, “Use of post-transcriptional gene silencing for identifying nucleic acid sequences that modulate the function of a cell”, the teaching of which is hereby incorporated by reference, can also be used in the present methods.

While the use of the present invention is not limited to vertebrate or mammalian cells, such cells can be used to carry out the methods described herein. Desirably, the vertebrate (e.g., mammalian) cells used to carry out the present invention are cells that have been cultured for only a small number of passages (e.g., less than 30 passages of a cell line that has been obtained directly from American Type Culture Collection), or are primary cells. In addition, vertebrate (e.g., mammalian) cells can be used to carry out the present invention when the dsRNA being transfected into the cell is not complexed with cationic lipids.

### Example 1

### Transcriptional and Post-Transcriptional Gene Silencing

Transcriptional gene silencing (TGS) is a phenomenon in which silencing of gene expression occurs at the level of RNA transcription. Double stranded RNA mediates TGS as well as post-transcriptional gene silencing (PTGS), but the dsRNA needs to be located in the nucleus, and desirably is made in the nucleus in order to mediate TGS. PTGS occurs in the cytoplasm. A number of dsRNA structures and dsRNA expression vectors have been delineated herein that can mediate TGS, PTGS, or both. Various strategies for mediating TGS, PTGS, or both are summarized below.

All of the cytoplasmic dsRNA expression vectors described herein mediate PTGS because they generate dsRNA in the cytoplasm where the dsRNA can interact with target mRNA. Because some of the dsRNA made by these vectors translocate to the nucleus via a passive process (e.g., due to nuclear envelope degeneration and reformation during mitosis), these vectors are also expected to affect TGS at a low efficiency in dividing cells. RNA PolII vectors express RNA molecules in the nucleus with various abilities to enter the cytoplasm.

If desired, one or more constitutive transport element (CTE) sequences can be added to enable cytoplasmic transport of the different effector RNA molecules (e.g., hairpins or duplexes) that are made in the nucleus by RNA PolII. A CTE can be used instead of and/or in addition to an intron and/or polyA sequence to facilitate transport. A desirable location for the CTE is near the 3′ end of the RNA molecules. If desired, multiple CTE sequences (e.g., 2, 3, 4, 5, 6, or more sequences can be used). A preferred CTE is from the Mason-Pfizer Monkey Virus (U.S. Pat. Nos. 5,880,276 and 5,585,263).

Vectors encoding a functional intron or CTE in combination with a polyadenylation signal more efficiently export dsRNA to the cytoplasm. Vectors with (i) only an intron or CTE and no polyadenylation signal, or (ii) with only a polyadenylation signal and no intron or CTE, export RNA to the cytoplasm with a lesser efficiency, resulting in less RNA in the cytoplasm and a lower efficiency for PTGS. Vectors encoding RNA without an intron, CTE, and polyadenylation signal result in RNA molecules that are the least efficiently transported to the cytoplasm. The lower the level of cytoplasmic transport of RNA, the more RNA retention in the nucleus and the higher efficiency with which TGS is induced. Therefore, all of these vectors induce PTGS and TGS with varying efficiencies according to the level of cytoplasmic transport and nuclear retention, respectively, as described above.

RNA Pol vectors, which can have one or more introns or no introns and can have a polyA tail or no polyA tail, encode RNA molecules that are made in the nucleus and are primarily retained in the nucleus. This nuclear RNA induces TGS. However, a percentage of the transcribed RNA reaches the cytoplasm and can therefore induce PTGS. For TGS induction, the dsRNA desirably contains a promoter, or a subset of a promoter sequence, and is retained in the nucleus. Alternatively, the dsRNA may contain only coding or UTR sequence, or may desirably contain a combination of coding or UTR sequence and promoter sequence. Such “fusion target” dsRNAs may contain, e.g., both a promoter sequence and a linked gene sequence to be targeted for concurrent TGS and PTGS. For PTGS, the dsRNA contains sequence derived from an RNA (e.g., coding or UTR sequence from an mRNA) and does not have to contain promoter sequence. In addition, more efficient PTGS is induced by vectors that enable cytoplasmic transcription or by vectors that result in more efficiently cytoplasmically transported RNA. If desired, PTGS and TGS can be induced simultaneously with a combination of these vectors using the methods described herein and techniques known to those skilled in the art.

Any of the vectors described in Example 11 (see below) or any other standard vector can also be used to generate the dsRNA structures of the invention, and used in the present methods.

### Example 2

### Exemplary Methods for Enhancing Post-Transcriptional Gene Silencing

To enhance PTGS by dsRNA transcribed in the nucleus by RNA PolII, one or more introns and/or a polyadenylation signal can be added to the dsRNA to enable processing of the transcribed RNA. This processing is desirable because both splicing and polyadenylation facilitate export from the nucleus to the cytoplasm. In addition, polyadenylation stabilizes RNA PolII transcripts. In some embodiments, a prokaryotic antibiotic resistance gene, e.g., a zeomycin expression cassette is located in the intron. Other exemplary prokaryotic selectable markers include other antibiotic resistance genes such as kanamycin, including the chimeric kanamycin resistance gene of U.S. Pat. No. 5,851,804, aminoglycosides, tetracycline, and ampicillin. The zeomycin gene is under the regulatory control of a prokaryotic promoter, and translation of zeomycin in the host bacterium is ensured by the presence of Shine-Dalgarno sequences located within about 10 base-pairs upstream of the initiating ATG. Alternatively, the zeomycin expression cassette can be placed in any location between the inverted repeat sequences of the hairpin (i.e., between the sense and antisense sequences with substantial identity to the target nucleic acid to be silenced).

Although inverted repeat sequences are usually deleted from DNA by DNA recombination when a vector is propagated in bacteria, a small percentage of bacteria may have mutations in the recombination pathway that allow the bacteria to stably maintain DNA bearing inverted repeats. In order to screen for these infrequent bacteria, a zeomycin selection is added to the culture. The undesired bacteria that are capable of eliminating inverted repeats are killed because the zeomycin expression cassette is also deleted during recombination. Only the desired bacteria with an intact zeomycin expression cassette survive the selection.

After the DNA is isolated from the selected bacteria and inserted into eukaryotes (e.g., mammalian cell culture) or into animals (e.g., adult mammals) for expression of RNA, the intron is spliced from the RNA transcripts. If the zeomycin expression cassette is located in the intron, this cassette is removed by RNA splicing. In the event of inefficient splicing, the zeomycin expression cassette is not expressed because there are no eukaryotic signals for transcription and translation of this gene. The elimination of the antibiotic resistance cassette is desirable for applications involving short dsRNA molecules because the removal of the cassette decreases the size of the dsRNA molecules. The zeomycin cassette can also be located beside either end of an intron instead of within the intron. In this case, the zeomycin expression cassette remains after the intron is spliced and can be used to participate in the loop structure of the hairpin. These RNA PolII transcripts are made in the nucleus and transported to the cytoplasm where they can effect PTGS. However, some RNA molecules may be retained in the nucleus. These nuclear RNA molecules may effect TGS. For TGS applications, the encoded dsRNA desirably contains a promoter or a subset of a promoter. In order to more efficiently retain RNA within the nucleus, the intron and/or polyadenylation signal can be removed.

Another strategy for both cytoplasmic and nuclear localization is to use “upstream” or internal RNA PolIII promoters (see, e.g., Gene regulation: A Eukaryotic Perspective, 3^{rd} ed., David Latchman (Ed.) Stanley Thornes: Cheltenham, UK, 1998). These promoters result in nuclear transcribed RNA transcripts, some of which are exported and some of which are retained in the nucleus and hence can be used for PTGS and/or TGS. These promoters can be used to generate hairpins, including the partial and forced hairpin structures of the invention, or duplex RNA through the use of converging promoters or through the use of a two vector or two cistronic system. One promoter directs synthesis of the sense strand, and the other promoter directs synthesis of the antisense RNA. The length of RNA transcribed by these promoters is generally limited to several hundred nucleotides (e.g., 250-500). In addition, transcriptional termination signals may be used in these vectors to enable efficient transcription termination.

Exemplary Vector Encoding a dsRNA with an Intron Containing an Antibiotic Resistance Gene

The human cytomegalovirus major immediate-early protein intron I (Accession No. M21295) was PCR amplified using the following forward primer KpnI-intron-f (5′-CGC GGG TAC CAA CGG TGC ATT GGA ACG C-3′) and the reverse primer NheI-intron-r (5′-ATC GGC TAG CGG ACG GTG ACT GCA GAA AAG ACC CAT GG-3′). These primers amplify the region from nucleotides 594 to 1469 and introduce a KpnI site on the 5′ end and a NheI site on the 3′ end of the intron. This product was inserted into the EcoRV site of pBSII KS(+) (Stratagene, LaJolla, Calif.) to create the vector pBS-IVS.

The Zeocin gene is commercially available (Invitrogen, pcDNA3.1(+)Zeo). The gene with a prokaryotic promoter was PCR amplified using the forward primer 5′ ZeoSphI (5′-ATG CAT GCC GTG TTG ACA ATT AAT CAT CGG C-3′) and the reverse primer 3′ ZeoHpaI (5′-ATG TTA ACC ACG TGT CAG TCC TGC TCC TCG-3′) using pcDNA (+Zeo) (Invitrogen). This PCR product was cleaved with SphI and HpaI, and the fragment was inserted into the hCMV intron A (Genbank accession number M21295, nucleotides 594-1470) contained at the SphI and HpaI sites to create the vector pBS-Iz. This insertion incorporates Zeocin into the intron A sequence in the same orientation and leaves the intron A acceptor and donor sites and their flanking regions intact (IVS-Zeocin).

The IVS-Zeocin (Iz) was excised from pBS-Iz using the enzymes KpnI and NheI and the isolated fragment was inserted into an expression vector downstream of a human cytomegalovirus promoter (Genbank accession number AF105229). Downstream of the insertion site, the vector contained the bovine growth hormone polyadenylation signal. The Iz was inserted into the KpnI and NheI sites of the vector MCS; this construct maintains the native orientation of Iz with respect to the promoter to allow for processing of the RNA and excision of the intronic sequence. The encoded RNA is also predicted to be polyadenylated. This vector was named pCMV-Iz.

Secreted Alkaline Phosphatase (SEAP; Genbank accession number U89938) was PCR amplified using the forward prime KpnI-SEAP-f (5′-AGC CGG TAC CCT ATT CCA GAA GTA GTG AGG-3′) and the reverse primer SEAP5′Xho (5′-CGT AAC TCG AGC ACT GCA TTC TAG TTG TGG-3′). This PCR reaction amplifies the full length SEAP and introduces a KpnI site into the 5′ end and a XhoI site into the 3′ end. The product was sub-cloned into pBSII KS(+) that was cleaved with EcoRI to create the vector pBS-SEAPKX. Full length SEAP was excised from pBS-SEAPKX using KpnI and XhoI and inserted into pCMV-Iz. This insertion was in the reverse orientation and was upstream of the Iz sequence using the KpnI and XhoI sites of the pCMV-Iz vector. A SEAPA PCR product was generated using the forward primer NheI-SEAP-f (5′-AGC CGC TAG CCT ATT CCA GAA GTA GTG AGG-3) and SEAP3′XhoI. This reaction produces a 650 base-pair fragment of SEAP with an NheI site on the 5′ end and an XhoI site on the 3′ end. The SEAP NheI/XhoI PCR product was cut with NheI and XhoI and inserted into pCMV-SEAP-Iz at the NheI and SalI restriction sites. This insertion was in the forward orientation and was downstream of the Iz sequence, generating the vector pCMV-SEAP-Iz-SEAPA. Selection on media containing 35 ug/ml Zeocin resulted in the successful replication of a vector containing a 650-700 base-pair inverted repeat. The replication of this desired vector occurred in DH5α cells but not in DH10B cells under the conditions tested.

This method has also been performed with mIL-12p40 (full length and 500 base-pair segments) and mCK-M. Additionally, this method was performed in two different vector systems utilizing both the T7 and the hCMV promoter system. Theoretically, this method can be performed for any vector, any promoter, any polyA signal, and any drug resistance gene or any positive selection marker inserted within or near any intron sequence that contains a functional acceptor and donor site.

### Example 3

### Exemplary Methods for the Generation of dsRNA In Vivo

Exemplary intracellular expression systems for sustained expression of dsRNA include cytoplasmic expression systems, e.g., a T7 promoter/T7 RNA polymerase, mitochondrial promoter/mitochondrial RNA polymerase, or RNA polII expression system. Other possible cytoplasmic expression systems use exogenously introduced viral or bacteriophage RNA polymerases and their cognate promoters or endogenous polymerases such as the mitochondrial RNA polymerase with their cognate promoters. In another embodiment, the sustained long dsRNA intracellular expression system is a nuclear expression system, such as an RNA polI, RNA polII, or RNA polIII expression system.

Constructs for Intracellular Expression of dsRNA in Vertebrate Cells

A variety of expression constructs capable of expressing dsRNA intracellularly in a vertebrate cell can be utilized to express the various at least partially double stranded RNA molecules, including forced and partial hairpin structures of the invention, and long dsRNA molecules having a double stranded region desirably at least 50 base-pairs, more desirably greater than 100 base-pairs, still more desirably greater than 200 base-pairs, including sequences of 1, 2, 3, 4, 5, or more kilobases that are within the maximum capacity for a particular plasmid, e.g., 20 kilobases, or as appropriate for a viral or other vector.

Expression vectors designed to produce dsRNA can be a DNA single stranded or double stranded plasmid or vector. Expression vectors designed to produce dsRNA as described herein may contain sequences under the control of any RNA polymerase, such as a mitochondrial RNA polymerase, RNA polII, RNA polIII, or exogenously introduced viral or bacteriophage RNA polymerase. Vectors may be desirably designed to utilize an endogenous mitochondrial polymerase (e.g., human mitochondrial RNA polymerase together with the corresponding human mitochondrial promoter). Mitochondrial polymerases may be used to generate capped dsRNA through expression of a capping enzyme or generate uncapped dsRNA transcripts in vivo. RNA polI, RNA polII, and RNA polIII transcripts may also be generated in vivo. Such RNA molecules may be capped or not, and if desired, cytoplasmic capping may be accomplished by various means including use of a capping enzyme such as a vaccinia capping enzyme or an alphavirus capping enzyme. DNA expression vectors are designed to contain one promoter or multiple promoters in combination (mitochondrial, RNA polI, RNA polII, RNA polIII, viral, bacterial or bacteriophage promoters) along with their cognate RNA polymerases (e.g., T3, T7, or SP6 bacteriophage systems). Desirably, RNA polII systems use a segment encoding a dsRNA that has an open reading frame greater than about 300 nucleotides to avoid degradation in the nucleus. Further information concerning constructs for the intracellular production of the RNA molecules of the invention, including viruses and viral sequences that may be manipulated to provide the required RNA molecule to the mammalian cell in vivo (e.g., alphavirus, adenovirus, adeno-associated virus, baculovirus, delta virus, pox viruses, hepatitis viruses, herpes viruses, papova viruses such as SV40, poliovirus, pseudorabies virus, retroviruses, vaccinia viruses, positive and negative stranded RNA viruses, viroids, and virusoids) can be found in, for example, WO 00/63364, which is incorporated herein by reference.

Any other DNA-dependent RNA polymerase (e.g., a viral, plant, invertebrate, or vertebrate polymerase) can be used (see, e.g., Table 2). In some embodiments, the dsRNA transcribed by the polymerase is expressed under the control of a promoter from the same organism, species, or genus from which the polymerase coding sequence was obtained.

TABLE 2
DNA dependent RNA polymerases

| | Genbank |
|---|---|
| Source of DNA-dependent RNA Polymerase | Accession No. |
| African swine fever virus NP1450L gene encoding | Z21489 |
| RNA polymerase largest subunit | |
| African swine fever virus, complete genome | NC_01659 |
| African swine fever virus, complete genome | U18466 |
| African swine fever virus EP1242L gene encoding | Z21490 |
| RNA polymerase second largest subunit | |
| Rabbit fibroma virus, complete genome | NC_001266 |
| Vaccinia virus, complete genome | NC_001559 |
| Autographa californica nucleopolyhedrovirus, complete | NC_001623 |
| genome | |
| Mastigamoeba invertens DNA-dependent RNA | AF083338 |
| polymerase II largest subunit (RPB1) gene, partial cds | |
| G. lamblia rpoA3 gene for subunit A of DNA dependent | X6032 |
| RNA polymerase III | |
| E. gracilis chloroplast RNA polymerase rpoB-rpoC1- | X17191 |
| rpoC2 operon | |
| Listeria monocytogenes unidentified gene and partial | Y16468 |
| rpoB gene | |
| Maize chloroplast RNA polymerase (rpoC1) gene, 5′ | M31207 |
| end | |
| Maize chloroplast RNA polymerase (rpoC2) gene, 5′ | M31208 |
| end | |
| Maize chloroplast RNA polymerase (rpoB) gene, 5′ end | M31206 |

Exemplary promoter and coding sequences of target nucleic acids are listed in Table 3 (see below). Other promoters and coding sequences can be readily identified by one skilled in the art from published databases or references or from standard methods such as standard sequence analysis techniques. For targeting a promoter, a dsRNA of, e.g., at least 19-30 nucleotides in length can be designed to include the TATA box or CAT box within the dsRNA (see, e.g., Molecular Cell Biology, Lodish (ed.) 3rd edition, Scientific American books: New York, 1995). In other embodiments, a region of, e.g., at least 350, 500, 750, 1000, 1500, 2000, or 2500 nucleotides upstream of the coding sequence can be used to target the promoter and/or other regulatory elements of a nucleic acid of interest. In certain desirable embodiments, both a promoter and a coding sequence will be targeted in the same dsRNA or dsRNA expression construct.

TABLE 3
Exemplary Target Genes and Promoters, and Genomes Containing Same

| | | Genbank |
|---|---|---|
| Virus | Target Genes and Promoters and Genomes containing same | Accession No. |
| Retroviruses | Human immunodeficiency virus type 2, complete genome | NC_001722.1 |
| | Human immunodeficiency virus type 1, complete genome | NC_001802.1 |
| | Human T-cell lymphotropic virus type 1, complete genome | NC_001436.1 |
| | Human T-cell lymphotropic virus type 2, complete genome | NC_001488.1 |
| Hepatitis B | Hepatitis B virus, complete genome | NC_003977.1 |
| Pox Viruses | Variola virus, complete genome | NC_001611.1 |
| | Vaccinia virus, complete genome | NC_001559.1 |
| Herpesvirus | Human herpesvirus 1, complete genome | NC_001806.1 |
| | Human herpesvirus 2, complete genome | NC_001798.1 |
| Epstein-barr Virus | Epstein-barr virus rna polmerase ii promoter region | J02075.1 |
| | Human herpesvirus 4, complete genome | NC_001345.1 |
| | Epstein-barr virus rna polymerase ii promoter region 12 | J02074.1 |
| | Epstein-barr virus (EBV) genome, strain B95-8 | V01555.1 |
| | Epstein-barr virus rna polymerase ii promoter region 11 | J02073.1 |
| Chicken pox | Human herpesvirus 3, complete genome | NC_001348 |
| Cytomegalovirus | Rat cytomegalovirus, complete genome | NC_002512.2 |
| | Chimpanzee cytomegalovirus, complete genome | NC_003521.1 |
| | Human herpesvirus 6, complete genome | NC_001664.1 |
| | Human herpesvirus 5, genome | NC_001347.1 |
| | Mouse cytomegalovirus 1, complete genome | NC_004065.1 |
| Human | Human papillomavirus type 1a, complete genome | NC_001356.1 |
| Papillomavirus | | |
| | Human papillomavirus type 2a, complete genome | NC_001352.1 |
| | Human papillomavirus type 4, complete genome | NC_001457.1 |
| | Human papillomavirus type 5b, complete genome | NC_001444.1 |
| | Human papillomavirus type 6, complete genome | NC_000904.1 |
| | Human papillomavirus type 8, complete genome | NC_001532.1 |
| | Human papillomavirus type 11, complete genome | NC_001525.1 |
| | Human papillomarvirus type 13, complete genome | NC_001349.1 |
| | Human papillomavirus tpe 16, complete genome | NC_001526.1 |
| | Human papillomavirus type 18, complete genome | NC_001357.1 |
| | Human papillomavirus type 31, complete genome | NC_001527.1 |
| | Human papillomavirus type 33, complte genome | NC_001528.1 |
| | Human papillmavirus type 35, complete genome | NC_001529.1 |
| | Human papillomavirus type 39, complete genome | NC_001535.1 |
| | Human papillomavirus type 41, complete genome | NC_001354.1 |
| | Human papillomavirus type 42, complete genome | NC_001534.1 |
| | Human papillomavirus type 47, complete genome | NC_001530.1 |
| | Human papillomavirus type 51, complete genome | NC_001533.1 |
| | Human papillomavirus type 57, complete genome | NC_001353.1 |
| | Human papillomavirus type 58, complete genome | NC_001443.1 |
| | Human papillomavirus type 63, complete genome | NC_001458.1 |
| | Human papillomavirus type 65, complete genome | NC_001459.1 |
| Adenovirus | Human adenovirus B, complete genome | NC_004001.1 |
| | Ovine adenovirus 7, complete genome | NC_004037.1 |
| | Porcine adenovirus C, complete genome | NC_002702.1 |
| | Bovine adenovirus A, complete genome | NC_002685.1 |
| | Murine adenovirus A, complete genome | NC_000942.1 |
| | Fowl adenovirus D, complete genome | NC_000899.1 |
| | Porcine adenovirus A, complete genome | NC_001997.1 |
| | Bovine adenovirus B, complete genome | NC_001876.1 |
| | Duck adenovirus A, complete genome | NC_001813.1 |
| | Canine adenovirus, complete genome | NC_001734.1 |
| | Human adenovirus A, complete genome | NC_001460.1 |
| | Human adenovirus F, complete genome | NC_001454.1 |
| | Human adenovirus C, complete genome | NC_001405.1 |
| | Fowl adenovirus A, complete genome | NC_001720.1 |
| | Ovine adenovirus A, complete genome | NC_002513.1 |
| | Human adenovirus D, complete genome | NC_002067.1 |
| | Human adenovirus E, complete genome | NC_003266.1 |
| | Frog adenovirus 1, complete genome | NC_002501.1 |
| | Hemorrhagic enteritis virus, complete genome | NC_001958 |
| ParvoVirus | Parvovirus H1, complete genome | NC_001358.1 |
| | Bovine parvovirus, complete genome | NC_001540.1 |
| | Porcine parvovirus strain NADL-2 | NC_001718.1 |
| | Canine parvovirus, complete genome | NC_001539.1 |
| | Goose parvovirus, complete genome | NC_001701.1 |
| | Aleutian mink disease parvovirus, complete genome | NC_001662.1 |
| | Mouse parvovirus 1, complete genome | NC_001630.1 |
| Other viruses | West Nile virus, complete genome | NC_001563.2 |
| | Japanese encephalitis virus (strain JaOArS982), complete | NC_001437.1 |
| | genome | |
| | Dengue virus type 2, complete genome | NC_001474.1 |
| | Dengue virus type 4, complete genome | NC_002640.1 |
| | Dengue virus type 1, complete genome | NC_001477.1 |
| | Dengue virus type 3, complete genome | NC_001475.1 |
| | Yellow fever virus, complete genome | NC_002031.1 |
| | Marburg virus, complete genome | NC_001608.2 |
| | Ebola virus, complete genome | NC_002549.1 |
| | Poliovirus, complete genome | NC_002058.3 |
| | Measles virus, complete genome | NC_001498.1 |
| | Mumps virus, complete genome | NC_002200.1 |
| Picornoviridae | Aichi virus, complete genome | NC_001918.1 |
| | Bovine enterovirus, complete genome | NC_001859.1 |
| | Human enterovirus 70, complete genome | NC_001430.1 |
| | Poliovirus, complete genome | NC_002058.3 |
| | Theiler's encephalomyelitis virus, complete genome | NC_001366.1 |
| | Porcine enterovirus A, complete genome | NC_003987.1 |
| | Foot-and-mouth disease virus SAT 2, genome | NC_003992.1 |
| | Foot-and-mouth disease virus C, complete genome | NC_002554.1 |
| | Equine rhinitis B virus, complete genome | NC_003983.1 |
| | Ljungan virus, complete genome | NC_003976.1 |
| | Human rhinovirus A, complete genome | NC_001617.1 |
| | Human rhinovirus B, complete genome | NC_001490.1 |
| | Hepatitis A virus, complete genome | NC_001489.1 |
| | Equine rhinovirus 3, complete genome | NC_003077.1 |
| | Porcine enterovirus B, complete genome | NC_001827.1 |
| | Human enterovirus A, complete genome | NC_001612.1 |
| | Human enterovirus B, complete genome | NC_001472.1 |
| | Human enterovirus C, complete genome | NC_001428.1 |
| | Human parechovirus 2, complete genome | NC_001897.1 |
| | Foot-and-mouth disease virus O, complete genome | NC_004004.1 |
| | Encephalomyocarditis virus, complete genome | NC_001479.1 |
| | A-2 plaque virus, complete genome | NC_003988.1 |
| | Avian encephalomyelitis virus strain | NC_003990.1 |
| | Mengo virus, complete genome | NC_003989.1 |
| | Human echovirus 1, complete genome | NC_003986.1 |
| | Porcine teschovirus, genome | NC_003985.1 |
| | Equine rhinitis A virus, complete genome | NC_003982.1 |
| Calcivirdae | Norwalk virus, complete genome | NC_001959.1 |
| | Calicivirus strain NB, complete genome | NC_004064.1 |
| | Rabbit hemorrhagic disease virus, complete genome | NC_001481.1 |
| | Feline calicivirus, complete genome | NC_001481.1 |
| | Porcine enteric calicivirus, complete genome | NC_000940.1 |
| | European brown hare syndrome virus, complete genome | NC_002615.1 |
| Astroviridae | Avian nephritis virus, complete genome | NC_003790.1 |
| | Human astrovirus, complete genome | NC_001943.1 |
| | Turkey astrovirus, complete genome | NC_002470.1 |
| | Sheep astrovirus, complete genome | NC_002469.1 |
| Togaviridae | Semliki forest virus, complete gename | NC_003215.1 |
| | Barmah Forest virus, complete genome | NC_001786.1 |
| | Mayaro virus, complete genome | NC_003417.1 |
| | Ross River virus, complete genome | NC_001544.1 |
| | Venezuelan equine encephalitis virus, complete genome | NC_001449.1 |
| | Rubella virus, complete genome | NC_001545.1 |
| | Sindbis virus, complete genome | NC_001547.1 |
| | O'nyong-nyong virus, complete genome | NC_001512.1 |
| | Igbo Ora virus, complete genome | NC_001924.1 |
| | Western equine encephalomyelitis virus, complete genome | NC_003908.1 |
| | Aura virus, complete genome | NC_003900.1 |
| | Salmon pancreas disease virus, complete genome | NC_003930.1 |
| | Eastern equine encephalitis virus, complete genome | NC_003899.1 |
| | Sleeping disease virus, complete genome | NC_003433.1 |
| Flavivirus | Hepatitis C virus, complete genome | NC_001433.1 |
| | Tamana bat virus, genome | NC_003996.1 |
| | West Nile virus, complete genome | NC_001563.1 |
| | Powassan virus, complete genome | NC_003687.1 |
| | Pestivirus Giraffe-1, complete genome | NC_003678.1 |
| | Pestivirus Reindeer-1, complete genome | NC_003677.1 |
| | Apoi virus, genome | NC_003676.1 |
| | Rio Bravo virus, genome | NC_003675.1 |
| | Pestivirus type 2, complete genome | NC_002657.1 |
| | Bovine viral diarrhea virus genotype 2, complete genome | NC_002032.1 |
| | Mosquito cell fusing agent, complete genome | NC_001564.1 |
| | Deer tick virus, genome | NC_003218.1 |
| | Louping ill virus, complete genome | NC_001809.1 |
| | Dengue virus type 2, complete genome | NC_001474.1 |
| | Yellow fever virus, complete genome | NC_002031.1 |
| | Dengue virus type 4, complete genome | NC_002640.1 |
| | Japanese encephalitis virus (strain JaOArS982), complete | NC_001437.1 |
| | genome | |
| | Langat virus, complete genome | NC_003690.1 |
| | Hepatitis GB virus C, complete genome | NC_002348.1 |
| | Dengue virus type 1, complete genome | NC_001477.1 |
| Coronaviridae | Transmissible gastroenteritis virus, complete genome | NC_002306.2 |
| | Murine hepatitis virus, complete genome | NC_001846.1 |
| | Bovine coronavirus, complete genome | NC_003045.1 |
| | Human coronavirus 229E, complete genome | NC_002645.1 |
| | Porcine epidemic diarrhea virus, complete genome | NC_003436.1 |
| | Avian infectious bronchitis virus, complete genome | NC_001451.1 |
| Rhabdoviridae | Rice yellow stunt virus, complete genome | NC_003746.1 |
| | Northern cereal mosaic virus, complete genome | NC_002251.1 |
| | Vesicular stomatitis virus, complete genome | NC_001560.1 |
| | Spring viremia of carp virus, complete genome | NC_002803.1 |
| | Bovine ephemeral fever virus, complete genome | NC_002526.1 |
| | Viral hemorrhagic septicemia virus, complete genome | NC_000855.1 |
| | Rabies virus, complete genome | NC_001542.1 |
| | Snakehead rhabdovirus, complete genome | NC_000903.1 |
| | Infectious hematopoietic necrosis virus, complete genome | NC_001652.1 |
| | Sonchus yellow net virus | NC_001615.1 |
| | Australian bat lyssavirus, complete genome | NC_003243.1 |
| Filoviridae | Marburg virus, complete genome | NC_001608.2 |
| | Ebola virus, complete genome | NC_002549.1 |
| Paramyxovirinae | Mumps virus, complete genome | NC_002200.1 |
| | Sendai virus, complete genome | NC_001552.1 |
| | Measles virus, complete genome | NC_001498.1 |
| | Human parainfluenza virus 1 strain Washington/1964, | NC_003461.1 |
| | complete genome | |
| | Newcastle disease virus, complete genome | NC_002617.1 |
| | Human parainfluenza virus 3, complete genome | NC_001796.2 |
| | Human parainfluenza virus 2, complete genome | NC_003443.1 |
| | Nipah virus, complete genome | NC_002728.1 |
| | Avian paramyxovirus 6, complete genome | NC_003043.1 |
| | Bovine parinfluenza virus 3, complete genome | NC_002161.1 |
| | Hendra virus, complete genome | NC_001906.1 |
| | Canine distemper virus, complete genome | NC_001921.1 |
| | Tupaia paramyxoviurs, complete genome | NC_002199.1 |
| Orthomyxoviridae | Influenza A virus RNA segment 1, complete sequence | NC_002023.1 |
| | Influenza A virus RNA segment 3, completed sequence | NC_002022.1 |
| | Influenza A virus RNA segment 2, complete sequence | NC_002021.1 |
| | Influenza A virus RNA segment 8, complete sequence | NC_002020.1 |
| | Influenza A virus RNA segment 5, complete sequence | NC_002019.1 |
| | Influenza A virus RNA segment 6, complete sequence | NC_002018 |
| | Influenza A virus RNA segment 4, complete sequence | NC_002017.1 |
| | Influenza A virus RNA segment 7, complete sequence | NC_002016.1 |
| | Influenza B virus RNA-1, completed sequence | NC_002204.1 |
| | Influenza B virus RNA-8, complete sequence | NC_002211.1 |
| | Influenza B virus RNA-7, complete sequence | NC_002210.1 |
| | Influenza B virus RNA-6, complete sequence | NC_002209.1 |
| | Influenza B virus RNA-5, complete sequence | NC_002208.1 |
| | Influenza B virus RNA-4, complete sequence | NC_002207.1 |
| | Influenza B virus RNA-3, complete sequence | NC_002206.1 |
| | Influenza B virus RNA-2, complete sequence | NC_002205.1 |
| Bunyaviridae | Watermelon spotted wilt segment S, complete sequence | NC_003843.1 |
| | Watermelon spotted wilt virus segment M, complete | NC_003841.1 |
| | sequence | |
| | Watermelon spotted wilt virus segment L, complete | NC_003832.1 |
| | sequence | |
| | Impatiens necrotic spot virus segment L, complete sequence | NC_003625.1 |
| | Impatiens necrotic spot virus segment S, complete sequence | NC_003624.1 |
| | Peanut bud necrosis virus segment M, complete sequence | NC_003620.1 |
| | Peanut bud necrosis virus segment S, complete sequence | NC_003619.1 |
| | Impatiens necrotic spot virus segment M, complete sequence | NC_003616.1 |
| | Peanut bud necrosis virus segment L, complete sequence | NC_003614.1 |
| | Rift Valley fever virus L segment, complete sequence | NC_002043.1 |
| | Bunyamwera virus L segment, complete sequence | NC_001925.1 |
| | Andes virus segment L, complete sequence | NC_003468.1 |
| | Andes virus segment M, complete sequence | NC_003467.1 |
| | Andes virus segment S, complete sequence | NC_003466.1 |
| | Tomato spotted wilt virus RNA-L, complete sequence | NC_002052.1 |
| | Tomato spotted wilt virus RNA-S, completed sequence | NC_002051.1 |
| | Tomato spotted wilt virus RNA-M, complete sequence | NC_002050.1 |
| | Rift Valley fever virus S segment, complete sequence | NC_002045.1 |
| | Rift Valley fever virus M, segment, complete sequence | NC_002044.1 |
| | Bunyamwera virus S segment, complete sequence | NC_001927.1 |
| | Bunyamwera virus M segment, complete sequence | NC_001926.1 |
| Arenaviridae | Ippy virus nucleocapsid protein gene, parital cds | IVU80003.1 |
| Lassa | Lassa virus glycoprotein precursor (GP) and nucleoprotein | AF333969.1 |
| | (NP) genes, complete cds | |
| | Lassa virus partial genomoic RNA for putative glycoprotein | AJ310764.1 |
| | precursor (gpc gene), isolate | |
| | Lassa virus strain AV glycoprotein precursor (GPC) and | AF246121.1 |
| | nucleoprotein (NP) genes, complete cds | |
| | Lassa virus strain las9608911 nucleoprotein gene, partial cds | AF182272.1 |
| | Lassa virus strain las803796 nucleoprotein gene, partial cds | AF182271.1 |
| | Lassa virus strain las808255 nucleoprotein gene, partial cds | AF182270.1 |
| | Lassa virus strain las807868 nucleoprotein gene, partial cds | AF182269.1 |
| | Lassa virus strain las807977 nucleoprotein gene, partial cds | AF182268.1 |
| | Lassa virus strain las807992 nuceloprotein gene, partial cds | AF182267.1 |
| | Lassa virus strain las803203 nucleoprotein gene, partial cds | AF182266.1 |
| | Lassa virus strain las807998 nucleoprotein gene, partial cds | AF182265.1 |
| | Lassa virus strain las806829 nucleoprotein gene, partial cds | AF182264.1 |
| | Lassa virus strain las803793 nucleoprotein gene, partial cds | AF182263.1 |
| | Lassa virus strain las803791 nuceloprotein gene, partial cds | AF182262.1 |
| | Lassa virus strain las806828 nucleoprotein gene, partial cds | AF182262 |
| | Lassa virus strain las803792 nucleoprotein gene, partial cds | AF182260 |
| | Lassa virus strain las803201 nucleoprotein gene, partial cds | AF182259.1 |
| | Lassa virus strain las803204 nuceloprotein gene, partial cds | AF182258.1 |
| | Lassa virus strain las807974 nucleoprotein gene, partial cds | AF182257.1 |
| | Lassa virus strain las803972 nucleoprotein gene, partial cds | AF182256 |
| Reoviridae | Rice dwarf virus segment 12, complete sequence | NC_003768.1 |
| | Rice dwarf virus segment 11, complete sequence | NC_003767.1 |
| | Rice dwarf virus segment 6, complete sequence | NC_003763.1 |
| | Rice dwarf virus segment 5, complete sequence | NC_003762.1 |
| | Rice dwarf virus segment 4, complete sequence | NC_003761.1 |
| | Rice dwarf virus segment 7, complete sequence | NC_003760.1 |
| | Rice dwarf virus segment 2, complete sequence | NC_003774.1 |
| | Rice dwarf virus segment 1, complete sequence | NC_003773.1 |
| | Rice dwarf virus segment 3, complete sequence | NC_003772.1 |
| | Rice ragged stunt virus segment 4, complete sequence | NC_003771.1 |
| | Rice ragged stunt virus segment 7, complete sequence | NC_003770.1 |
| | Rice ragged stunt virus segment 10, complete sequence | NC_003769.1 |
| | Rice ragged stunt virus segment 5, complete sequence | NC_003759.1 |
| | Rice ragged stunt virus segment 8, complete sequence | NC_003758.1 |
| | Rice ragged stunt virus segment 9, complete sequence | NC_003757.1 |
| | Rice ragged stunt virus segment 6, complete sequence | NC_003752.1 |
| | Rice ragged stunt virus segment 3, complete sequence | NC_003751.1 |
| | Rice ragged stunt virus segment 2, complete sequence | NC_003750.1 |
| | Rice ragged stunt virus segment 1, complete sequence | NC_003749.1 |
| | Rice black streaked dwarf virus segment 6, complete | NC_003737.1 |
| | sequence | |
| | Rice black streaked dwarf virus segment 5, complete | NC_003736.1 |
| | sequence | |
| | Rice black streaked dwarf virus segment 4, complete | NC_003735.1 |
| | sequence | |
| | Rice black streaked dwarf virus segment 2, complete | NC_003734.1 |
| | sequence | |
| | Rice streaked dwarf virus segment 10, complete sequence | NC_003733.1 |
| | Rice black streaked dwarf virus segment 8, complete | NC_003732.1 |
| | sequence | |
| | Rice black streaked dwarf virus segment 9, complete | NC_003731.1 |
| | sequence | |
| | Rice black streaked dwarf virus segment 7, complete | NC_003730.1 |
| | sequence | |
| | Rice black streaked dwarf virus segment 1, complete | NC_003729.1 |
| | sequence | |
| | Rice black streaked dwarf virus segment 3, complete | NC_003728.1 |
| | sequence | |
| | Eyach virus segment 12, complete sequence | NC_003707.1 |
| | Eyach virus segment 11, complete sequence | NC_003706.1 |
| | Eyach virus segment 10, complete sequence | NC_003705.1 |
| | Eyach virus segment 9, complete sequence | NC_003704.1 |
| | Eyach virus segment 8, complete sequence | NC_003703.1 |
| | Eyach virus segment 7, complete sequence | NC_003702.1 |
| | Eyach virus segment 6, complete sequence | NC_003701.1 |
| | Eyach virus segment 5, complete sequence | NC_003700.1 |
| | Eyach virus segment 4, complete sequence | NC_003699.1 |
| | Eyach virus segment 3, complete sequence | NC_003698.1 |
| | Eyach virus segment 2, complete sequence | NC_003697.1 |
| | Eyach virus segment 1, complete sequence | NC_003696.1 |
| | Nilaparvata lugens reovirus segment 9, complete sequence | NC_003661.1 |
| | Nilaparvata lugens reovirus segment 7, complete sequence | NC_003660.1 |
| | Nilaparvata lugens reovirus segment 6, complete sequence | NC_003659.1 |
| | Nilapravata lugens reovirus segment 5, complete sequence | NC_003658.1 |
| | Nilaparvata lugens reovirus segment 4, complete sequence | NC_003657.1 |
| | Nilaparvata lugens reovirus segment 3, complete sequence | NC_003656.1 |
| | Nilaparvata lugens reovirus segment 2, complete sequence | NC_003655.1 |
| | Nilaparvata lugens reovirus segment 1, complete sequence | NC_003654.1 |
| | Nilaparvata lugens reovirus segment 8, complete sequence | NC_003653.1 |
| | Nilaparvata lugens reovirus segment 10, complete sequence | NC_003652.1 |
| | Lymantria dispar cypovirus 1 segment 10, complete | NC_003025.1 |
| | sequence | |
| | Lymantria dispar cypovirus 1 segment 9, complete sequence | NC_003024.1 |
| | Lymantria dispar cypovirus | NC_003023.1 |
| | 1 segment 8, complete sequence | |
| | Lymantria dispar cyporvirus 1 segment 7, complete virus | NC_003022.1 |
| | Lymantria dispar cyporvirus 1 segment 6, complete sequence | NC_003021.1 |
| | Lymantria dispar cypovirus | NC_003020.1 |
| | 1 segment 5, complete sequence | |
| | Lymantria dispar cyporivus 1 segment 4, complete sequence | NC_003019.1 |
| | Lymantria dispar cypovirus 1 segment 3, complete sequence | NC_003018.1 |
| | Lymantria dispar cypovirus 1 segment 2, complete sequence | NC_003017.1 |
| | Lymantria dispsar cypovirus 1 segment 1, complete | NC_003016.1 |
| | sequence | |
| | Lymantria dispar cypovirus 14 segment 10, complete | NC_003015.1 |
| | sequence | |
| | Lymantria dispar cypovirus | NC_003014.1 |
| | 14 segment 9, complete sequence | |
| | Lymantria dispar cypovirus 14 segment 8, complete | NC_003013.1 |
| | sequence | |
| | Lymantria dispar cypovirus 14 segment 7, complete | NC_003012.1 |
| | sequence | |
| | Lymantria dispar cypovirus 14 segment 6, complete | NC_003011.1 |
| | sequence | |
| | Lymantria dispar cypovirus 14 segment 5, complete | NC_003010.1 |
| | sequence | |
| | Lymantria dispar cypovirus 14, segment 4, complete | NC_003009.1 |
| | sequence | |
| | Lymantria dispar cypovirus 14 segment 3, complete | NC_003008.1 |
| | sequence | |
| | Lymantria dispar cypovirus 14 segment 2, complete virus | NC_003007.1 |
| | Lymantria dispar cypovirus 14 segment 1, complete | NC_003006.1 |
| | sequence | |
| | Trichoplusia ni cytoplasmic polyhedrosis virus 15 segment | NC_002567.1 |
| | 4, complete sequence | |
| | Trichoplusia ni cytoplasmic polyhedrosis virus 15 segment | NC_002566.1 |
| | 11, complete sequence | |
| | Trichoplusia ni cytoplasmic polyhedrosis virus 15 segment | NC_002565.1 |
| | 10, complete sequence | |
| | Trichoplusia ni cytoplasmic polyhedrosis virus 15 segment | NC_002564.1 |
| | 9, complete sequence | |
| | Trichoplusia ni cytoplasmic polyhedrosis virus 15, segment | NC_002563.1 |
| | 8, complete sequence | |
| ** | Trichoplusia ni cytoplasmic polyhedrosis virus 15 segment | NC_002562.1 |
| | 7, complete sequence | |
| | Trichoplusia ni cytoplasmic polyhedrosis virus 15 segment | NC_002561.1 |
| | 6, complete sequence | |
| | Trichoplusia ni cytoplasmic polyhedrosis virus 15 segment | NC_002560.1 |
| | 5, complete sequence | |
| | Trichoplusia ni cytoplasmic polyhedrosis virus 15 segment | NC_002559.1 |
| | 3, complete sequence | |
| | Trichoplusia ni cytoplasmic polyhedrosis virus 15, segment | NC_002558.1 |
| | 2, complete sequence | |
| | Trichoplusia ni cytoplasmic polyhedrosis virus 15, segment | NC_002557.1 |
| | 1, complete sequence | |
| Prion | Homo sapiens mRNA for prion protein, complete cds | D00015.1 |
| | Human prion protein 27-30 mRNA, complete cds | M13667 |
| | Homo sapiens prion protein (p27-30) (Creutzfled-Jakob | NM_000311 |
| | disease, Gerstmann-Strausler-Scheinker syndrome, fatal | |
| | familial insomnia) (PRNP), mRNA | |
| | MAJOR PRION PROTEIN PRECURSOR (PRP) (PRP27-30) | P52114 |
| | (PRP33-35C) | |
| | prion protein [Mustela putorius] | AAA69022 |
| | prion protein [mink, Genomic, 2446 nt] | S46825 |
| | Major prion protein precursor (PrP) (PrP27-30) (PrP33-35C) | P04156 |
| | (ASCR) (CD230 antigen) | |
| | Odocoileus virginianus prion protein precursor (PrP) gene, | AF156185 |
| | complete cds | |
| | Odocoileus virginianus prion protein precursor (PrP) gene, | AF156186 |
| | PrP-96Gly-138Asn allele, partial cds | |
| | Cervus elaphus nelsoni prion protein precursor (PrP) gene, | AF156182 |
| | PrP-132L allele, complete cds | |
| | Antilocapra Americana prion protein precursor (PrP) gene, | AF156187 |
| | complete cds | |
| | Cervus elaphus nelsoni prion protein precursor (PrP) gene, | AF156183 |
| | complete cds | |
| | Odocoileus virginianus prion protein precursor (PrP) gene, | AF156184 |
| | PrP-96Ser allele, complete cds | |
| | Felis catus prion protein (Prp) gene, complete cds | AF003087.1 |
| | Sheep gene for protein PrP, complete cds | D38179.1 |
| | Bos taurus prp gene for prion protein | AJ298878 |
| | Bos taurus mRNA for prion protein, complete cds | AB001468 |
| | Bovine mRNA for prion protein | D10612 |
| | Capra hircus prion protein (PrP) gene, complete cds | S82626 |
| | Homo sapiens v-abl Abelson murine leukemia viral | XM_033355.1 |
| | oncogene homology 1 | |
| | Mus musculus similar to Proto-oncogene tyrosine-protein | XM_130089 |
| | kinase ABL1 (p150) (c-ABL) (LOC227716), mRNA | |
| B-Raf | Homo sapiens v-raf murine sarcoma viral oncogene | NM_004333 |
| | homology B1 (BRAF), mRNA | |
| | H. sapiens B-raf-1 gene for 94 kDa B-raf protein | X65187.1 |
| | Mus musculus similar to B-Raf proto-oncogene | XM_133086 |
| | serin/threoine-protein kinase (p94) (v-Raf murine sarcoma | |
| | viral oncogene homolog B1) (LOC232705), mRNA | |
| | Mus musculus WGS supercontig Mm6_WIFeb01_98 | NW_000273 |
| BCL1 | H. sapiens of BCL1 mRNA encoding cyclin | Z23022 |
| | Homo sapiens genomic DNA, chromosome 11q, clone: | AP001824 |
| | CTD-2507F7, complete sequence | |
| | H. sapiens cyclin D1 gene promoter region | Z29078 |
| BCL-2 | Homo sapiens BCL-2 antagonist of cell death (BAD) | NM_004322 |
| | transcript variant 1, mRNA | |
| | Homo sapiens Bcl-X/Bcl-2 binding protein (BAD) mRNA, | AF021792 |
| | partial cds | |
| | Homo sapiens v-raf-1 murine leukemia viral oncogene | NM_002880.1 |
| | homolog 1 (RAF1), mRNA | |
| | Homo sapiens BCL-2 antagnoist of cell death (BAD) | NM_032989.1 |
| | transcript variant 2, mRNA | |
| BCL-6 | Human zinc-finger protein (bcl-6) mRNA, complete cds | UO0115 |
| CBFA2 | Human AML1 mRNA for AML1c protein (alternatively | XM_003789 |
| | spliced product), complete cds | |
| CSF1R | Homo sapiens colony stimulating factor 1 receptor, formerly | XM_003789 |
| | McDonough feline sarcoma viral (v-fms) oncogene | |
| | homology (CSF1R), mRNA | |
| | Homo sapiens choromosome 5 working draft segment | NT_006859 |
| EGFR | Human epidermal growth factor (beta-urogastrone) gene | J02548.1 |
| | (synthetic) | |
| | Homo sapiens epidermal growth factor (beta-urogastrone) | NM_001963.2 |
| | (EGF), mRNA | |
| ERB-B-2 | Human tyrosine kinase-type receptor (HER2) mRNA, | M11730.1 |
| | complete cds | |
| | Human c-erb-B-mRNA | X03363 |
| FOS | Human cellular oncogene c-fos (complete sequence) | V01512 |
| | Human fos proto-oncogene (c-fos), complete cds | K00650 |
| | Homo sapiens v-fos FBJ murine osteosarcoma viral | NM_005252.2 |
| | oncogene homology (FOS), mRNA | |
| HRAS | Human (genomic clones lambda-[SK2-T2-HS578T]; cDNA | J00277 |
| | clones RS-[3,4,6]) c-Ha-ras 1 proto-oncogene, complete | |
| | coding sequence | |
| | Homo sapiens, Similar to v-Ha-ras Harvey rat sarcoma viral | BC006499.1 |
| | oncogene homology, clone MGC: 2359 IMAGE: 2819996, | |
| | mRNA, complete cds | |
| Myb | Human c-myb mRNA, complete cds | M15024.1 |
| | Homo sapiens v-myb myeloblastosis viral oncogene | NM_005375.1 |
| | homology (avian) (MYB), mRNA | |
| | Human c-myb mRNA, complete cds | M15024.1 |
| | Human (c-myb) gene, complete primary cds, and five | HSU22376.1 |
| | complete alternatively spliced cds | |
| | c-myb {promoter, 5′ region} [human, leukocytes, Genomic, | S66422.1 |
| | 1284 nt] | |
| Myc | Human mRNA encoding the c-myc oncogene | V00568.1 |
| | Homo sapiens v-myc myelocytomatosis viral oncogene | NM_002467 |
| | homolog (avian) (MYC), rRNA | |
| | Homo sapiens MYC gene for c-myc proto-oncogene and | X00364.2 |
| | ORF-1 | |
| | Human c-myc-P64 mRNA, initiating from promoter P0, | M13930.1 |
| | (Hlmyc3.1) partial cds | |
| LCK | Human lck mRNA for membrane associated protein tyrosine | X13529.1 |
| | kinase | |
| | Human lymphocyte-specific protein tyrosine kinase (lck) | M36881.1 |
| | mRNA, complete cds | |
| | Homo sapiens lymphocyte-specific protein tyrosine kinase | M26693.1 |
| | (LCK) gene, exon2 and upstream promoter region | |
| | Human mutant lymphocyte-specific protein tyrosine kinase | U07236.1 |
| | (LCK) mRNA, complete cds | |
| | Human T-lymphocyte specific protein tyrosine kinase p56lck | U23852 |
| | (lck) abberant mRNA, complete cds | |
| | homo sapiens, clone MGC: 17196 IMAGE: 4341278, mRNA, | BC013200.1 |
| | complete cds | |
| MYCL1 | Homo sapiens v-myc myelocytomatosis viral oncogene | NM_005376.1 |
| | homology, lung carcinoma derived (avian-(MYCL1), | |
| | mRNA | |
| MYCN | Homo sapiens v-myc myelocytomatosis viral related | NM_005378 |
| | oncogene, neuroblastoma derived (avian_(MYCN), mRNA | |
| | Homo sapiens truncated MYCN fusion protein (MYCN) | AF317388 |
| | gene, complete cds | |
| NRAS | Rattus norvegicus Neuroblastoma RAS viral (v-ras_oncogene | NM_080766.1 |
| | homolog (Nras), mRNA | |
| | Mus musculus WGS supercontig Mm3_WIFeb01_50 | NW_000200.1 |
| | Mus musculus neuroblastoma ras oncogene (Nras), mRNA | XM_124137.1 |
| | Homo sapiens chromosome 1 working draft sequence | NT_019273.11 |
| | segment | |
| | Homo sapiens neuroblastoma RAS viral (v-ras) oncogene | XM_032698.6 |
| | homolog (NRAS), mRNA | |
| | G-15 Mouse, 4 months old female, left ventricular cardiac | BM658481.1 |
| | muscle cells cDNA library Mus musculus cDNA similar to | |
| | Mus musculus neuroblastoma ras oncogene (Nras), | |
| | Homo sapiens Ras family small GTP binding protein N-Ras | AF493919.1 |
| | (NRAS) mRNA, complete cds | |
| ROSI | Mus musculus Ros1 proto-oncogene (Ros1), mRNA | XM_125632 |
| | Homo sapiens chromosome 6 working draft sequence | NT_033944 |
| | segment | |
| | Homo sapiens v-ros UR2 sarcoma virus oncogene homolog | NM_002944.2 |
| | 1 (avian) (ROS1), mRNA | |
| | Human c-ros-1 proto-oncogene | AH002964.1 |
| RET | RET = proto-oncogene [human, neuroblastoma cell line LA- | S80097 |
| | 0N-2, 3621 nt] | |
| | Homo sapiens v-src sarcoma (Schmidt-Ruppin A-2) viral | NM_005417.2 |
| | oncogene homolog (avian) | |
| TCF3 | Human transcription factor (ITF-1) mRNA, 3′ end | X52078.1 |
| | Human e12 protein (E2A) mRNA, complete cds | M31222 |
| | Human (HeLa) helix-loop-helix protein HE47 (E2A) | M65214.1 |
| | mRNA, 3′ end | |
| | Human transcription factor (E2A) mRNA, complete cds | M31523.1 |

T7 Promoter/T7 Polymerase Expression Systems

A desirable method of the invention utilizes a T7 dsRNA expression system to achieve cytoplasmic expression of dsRNA, (e.g., long or short dsRNA molecules) in vertebrate cells (e.g., mammalian cells). Intracellular expression of short dsRNA molecules is expected to increase the duration of the silencing with respect to exogenously added short dsRNA molecules. The T7 expression system utilizes the T7 promoter to express the desired dsRNA. Transcription is driven by the T7 RNA polymerase, which can be provided on a second plasmid or on the same plasmid. For example, a first plasmid construct that expresses both a sense and antisense strand under the control of converging T7 promoters and a second plasmid construct that expresses the T7 RNA polymerase under the control of an RSV promoter can be used. Both the dsRNA and the T7 RNA polymerase could advantageously be expressed from a single bicistronic plasmid construct, particularly when the dsRNA is formed from a single RNA strand with inverted repeats or regions of self-complementarity that enable the strand to assume a stem-loop or hairpin structure with an at least partially double stranded region Individual sense and antisense strands which self assemble to form a dsRNA can be synthesized by a single plasmid construct using, e.g., converging promoters such as bacteriophage T7 promoters placed respectively at the 5′ and 3′ ends of the complementary strands of a selected sequence to be transcribed.

### Example 4

### Exemplary Methods for the Generation of dsRNA In Vitro

Short and long dsRNA can be made using a variety of methods known to those of skill in the art. For example, ssRNA sense and antisense strands, or single RNA strands with inverted repeats or regions of self-complementarity that enable the strand to assume a stem-loop or hairpin structure with an at least partially double stranded region, including the hairpin structures of the invention, can be synthesized chemically in vitro (see, for example, Q. Xu et al, Nucl. Acids. Res., 24 (18): 3643-3644, 1996 and other references cited in WO 00/63364, pp. 16-7), transcribed in vitro using commercially available materials and conventional enzymatic synthetic methods, (e.g., using the bacteriophage T7, T2, or SP6 RNA polymerases according to conventional methods such as those described by Promega Protocols and Applications Guide 3rd Ed., Eds. Doyle, 1996, ISBN No. 1-882274-57-1), or expressed in cell culture using recombinant methods. The RNA can then be purified using non-denaturing methods inducing various chromatographic methods and hybridized to form dsRNA. Such methods are well known to those of skill in the art and are described, for example, in WO 01/75164, WO 00/63364, and Sambrook et al., Molecular Cloning, A Laboratory Manual, 2^{nd} Ed.; Cold Spring Harbor Laboratory Press, New York, 1989, the teaching of which is incorporated herein by reference.

In vitro transcription reactions are carried out using the Riboprobe Kit (Promega Corp.), according to the manufacturer's directions. The template DNA is as described above. Following synthesis, the RNA is treated with Proteinase K and extracted with Phenol-chloroform to remove contaminating RNases. The RNA is ethanol precipitated, washed with 70% ethanol, and resuspended in RNase-free water. Aliquots of RNA are removed for analysis and the RNA solution is flash frozen by incubating in an ethanol-dry ice bath. The RNA is stored at −80% C.

As an alternative to phenol-chloroform extraction, RNA can be purified in the absence of phenol using standard methods such as those described by Li et al. (WO 00/44943, filed Jan. 28, 2000). Alternatively, RNA that is extracted with phenol and/or chloroform can be purified to reduce or eliminate the amount of phenol and/or chloroform. For example, standard column chromatography can be used to purify the RNA (WO 00/44914, filed Jan. 28, 2000).

Double stranded RNA can be made by combining equimolar amounts of PCR fragments encoding antisense RNA and sense RNA, as described above, in the transcription reaction. Single stranded antisense or sense RNA is made by using single species of PCR fragment in the reaction. The RNA concentration is determined by spectrophotometric analysis, and RNA quality is assessed by denaturing gel electrophoresis and by digestion with RNase T1, which degrades single stranded RNA.

If desired, an mRNA library is produced using Qbeta bacteriophage, by ligating the mRNA molecules to the flank sequences that are required for Qbeta replicase function (Qbeta flank or Qbeta flank plus P1), using RNA ligase. The ligated RNA molecules are then transformed into bacteria that express Qbeta replicase and the coat protein. Single plaques are then inoculated into fresh bacteria. All plaques are expected to carry transgene sequences. Each plaque is grown in larger quantities in bacteria that produce the Qbeta polymerase, and RNA is isolated from the bacteriophage particles. Alternatively, if the Qbeta flank plus P1 is used to generate the library (e.g., P1=MS2, VEEV, or Sindbis promoter sequences), these vectors can be used to carry out the in vitro transcription along with the cognate polymerase. The in vitro made dsRNA is then used to transfect cells.

### Example 5

### Exemplary Constructs that Enable the Efficient Formation of Hairpin dsRNA In Vivo or In Vitro

Constructs encoding a unimolecular hairpin dsRNA are more desirable for some applications than constructs encoding duplex dsRNA (i.e., dsRNA composed of one RNA molecule with a sense region and a separate RNA molecule with an antisense region) because the single-stranded RNA with inverted repeat sequences more efficiently forms a dsRNA hairpin structure. This greater efficiency is due in part to the occurrence of transcriptional interference arising in vectors containing converging promoters that generate duplex dsRNA. Transcriptional interference results in the incomplete synthesis of each RNA strand thereby reducing the number of complete sense and antisense strands that can base-pair with each other and form duplexes. Transcriptional interference can be overcome, if desired, through the use of (i) a two vector system in which one vector encodes the sense RNA and the second vector encodes the antisense RNA, (ii) a bicistronic vector in which the individual strands are encoded by the same plasmid but through the use of separate cistrons, or (iii) a single promoter vector that encodes a hairpin dsRNA, i.e., an RNA in which the sense and antisense sequences are encoded within the same RNA molecule. Hairpin-expressing vectors have some advantages relative to the duplex vectors. For example, in vectors that encode a duplex RNA, the RNA strands need to find and base-pair with their complementary counterparts soon after transcription. If this hybridization does not happen, the individual RNA strands diffuse away from the transcription template and the local concentration of sense strands with respect to antisense strands is decreased. This effect is greater for RNA that is transcribed intracellularly compared to RNA transcribed in vitro due to the lower levels of template per cell. Moreover, RNA folds by nearest neighbor rules, resulting in RNA molecules that are folded co-transcriptionally (i.e., folded as they are transcribed). Some percentage of completed RNA transcripts is therefore unavailable for base-pairing with a complementary second RNA because of intra-molecular base-pairing in these molecules. The percentage of such unavailable molecules increases with time following their transcription. These molecules may never form a duplex because they are already in a stably folded structure. In a hairpin RNA, an RNA sequence is always in close physical proximity to its complementary RNA. Since RNA structure is not static, as the RNA transiently unfolds, its complementary sequence is immediately available and can participate in base-pairing because it is so close. Once formed, the hairpin structure is predicted to be more stable than the original non-hairpin structure. Particularly preferred RNA hairpins are the “forced” hairpin constructs and “partial” hairpin constructs as described herein.

Forced Hairpin Constructs

“Forced” hairpin constructs are desirable because they are more efficiently expressed in a stem-loop or hairpin structure. These hairpin expression vectors encode a sequence referred to here as “Sequence A,” located soon after the “sense or antisense RNA” sequence of the selected target (FIGS. 1A-1D). Sequence A is also located before the loop of the hairpin and is complementary to a sequence referred to here as “Sequence B,” which is located after the loop and before the “antisense or sense RNA” sequence of the selected target. Desirably, Sequence A and Sequence B do not contain any self-complementarity. Desirably, the nucleotides of Sequence A are primarily G or primarily C, and the nucleotides of Sequence B are primarily C or primarily G, respectively, such that sequence A and sequence B are complementary to each other. For example, Sequence A can be GGGGGGGGGGG or GGGT(U)GGGGT(U)GGG (note that the designation T(U) refers to the thymidine (T) residue present in the DNA sequence, while uridine (U) is present in the encoded dsRNA hairpin). In this case, sequence B can be, e.g., CCCCCCCCCCC or CCCACCCCACCC, respectively. Monosequences of G's or C's may be desirable, as are sequences which are primarily C or primarily G, or even a string of alternating CGCGCGC . . . or GCGCGCG . . . bases may be used, as well as other similar variations, so long as sequences A and B are complementary to each other, and do not themselves have any significant secondary structure, or exhibit any significant base-pairing with other sequences such as the loop, the sense or antisense of the target region, or any other sequences within the transcript. In some aspects, especially when expressing short hairpin RNAs, e.g., 19 to 30 base-pairs, 19 to 27 base-pairs, or 19 to 23 base-pairs in length, A and B sequences may be selected to have less than 4 contiguous nucleotides that will base-pair with any other sequences such as the loop, the sense or antisense of the target region, or any other sequences within the transcript. In hairpins containing longer sequences of interest, e.g., a hundred to hundreds of base-pairs, in some aspects A and B sequences may be selected to have less than 7 contiguous nucleotides that will base-pair with any other sequences such as the loop, the sense or antisense of the target region, or any other sequences within the transcript. The complementarity between sequence A and B drives hairpin formation because RNA folds by nearest neighbor rules and because there is no self-complementarity in Sequence A or in Sequence B. Increasingly efficient hairpin formation is driven as the delta G of the base-paired structure between Sequences A and B becomes lower. This increased efficiency can be achieved by increasing the number of G/C base-pairs and/or by increasing the total number of base-pairs in sequence A and B. Sequence A and Sequence B are desirably between 10 base-pairs to 100 base-pairs, more desirably between 10 and 15 base-pairs, and less desirably between 4 and 10 base-pairs or between 100-200 base-pairs.

Between sequence A and sequence B is a sequence of minimally at least about 4 to 7 nucleotides, desirably about 7 to 15, e.g., 7, 8, 9, 10, 11, 12, 13, 14, and 15, or between 7 and 25, and maximally several hundred nucleotides which will serve as the loop. When loop structures are designed to encode other functionalities, e.g., a ribozyme (FIG. 7D), the number of nucleotides in the loop will be correspondingly greater to accommodate this functionality. In general, a monosequence of T's (T's in the DNA vector; U's in the transcribed RNA) or A's is desirable in the loop of a forced hairpin. Since pyrimidines may be preferred in the loop because of their greater susceptibility to digestion by nucleases, a monosequence of T's (T's in the DNA vector; U's in the transcribed RNA) may be desirable in hairpins. Constructs comprising a U6 promoter are an exception since a sequence of 4 to 5 T's serves as a terminator in U6 systems. In general, for loop sequences of forced hairpins, the following order of nucleotide preference may be considered: T (U)>A>C>G. Sequences containing a string of G's are generally to be avoided in loop structures. Loop sequences such as CACACA . . . , ACACAC . . . , T(U)T(U)CT(U)T(U)C . . . , or CT(U)T(U)CT(U)T(U) . . . may also be used, as may other similar variations. Some degree of secondary structure within the loop is allowed if it is not significant. In transcripts which do not contain multiple G's, as found in, e.g., some “forced” hairpins, C's as well as T's (U's) (i.e., pyrimidines) are preferred in loop structures. In some aspects, in short hairpins, the loop structure may be selected to have less than 4 contiguous nucleotides that will base-pair with any other sequences, such as the sense or antisense of the target region, or any other sequences within the transcript. In hairpins containing longer sequences of interest, e.g., hundreds of base-pairs, in some aspects loop sequences may be selected to have less than 7 contiguous nucleotides that will base-pair with any other sequences, such as the sense or antisense of the target region, or any other sequences within the transcript.

### Example 6

### Generation of Partial Hairpins Capable of Being Extended by RNA-Dependent RNA Polymerase to Form dsRNA Hairpins

Hairpin formation is efficiently driven by extension of a partial hairpin. A partial hairpin is a hairpin that does not contain a full complementary sequence with respect to Region 1 (which may be either antisense or sense with respect to the selected target, or which may include a sequence of target-specific sense or antisense followed by a sequence designed to “force” hairpin formation such as the “A” region of FIG. 1; see also, e.g., FIG. 2A). A complete hairpin cannot be formed by virtue of the fact that only an incomplete complementary sequence is present in the molecule. The partial hairpin can be self-extended and converted into a complete hairpin in the presence of an RNA-dependent RNA polymerase activity in vivo or in vitro. For in vivo use, RNA-dependent RNA polymerase activity can be endogenous polymerase activity such as the activity described by Chang and Taylor (EMBO J, pp. 157-164, 2002) or can be provided by transfection/infection of an expression plasmid/viral vector encoding an exogenous source of an RNA-dependent RNA polymerase such as a polymerase encoded by a variety of RNA viruses including, but not limited to, alphaviruses. RNA-dependent RNA polymerases and/or their cDNA sequences may be obtained from a variety of species including viruses, plants, invertebrates, and vertebrates (e.g., mammals or humans). Extension of the partial hairpin occurs by copying the Region 1 template in a transcription/replication reaction in vitro or in viva. Extension of the partial hairpin therefore requires the complement to the growing hairpin to act as a template for extension. Copying of the template through replication or transcription unfolds the Region 1 template thereby making it available for base-pairing with the newly extended hairpin since the hairpin is now a nearest neighbor.

In order to generate partial hairpins that are capable of being self-extended, the 3′ terminal nucleotide(s) of the hairpin need to be base-paired with the template so that the hairpin can be extended by an RNA-dependent RNA polymerase. Desirably, at least six nucleotides, desirably at least five to fifteen nucleotides, at the 3′ end are base-paired with the template part of the RNA. The following base pairs are listed in order of decreasing preference for this 3′ terminal/template region: G:C>A:U=A:C>G:U. The following based-pairs for stem structures (e.g., nucleotides farther from the 3′ terminus) are also listed in order of decreasing preference: G:C>A:G>AU A: AC>G:U.

Desirably, transcription termination occurs at a pre-fixed nucleotide or within a pre-defined set of nucleotides. Alternatively, a construct can be designed such that the RNA is processed (i.e., cleaved) at a defined sequence to generate the correct sequence at the 3′ end. If the RNA is not cleaved at the correct sequence, the 3′ terminal nucleotides may not be able to base-pair with the template in the appropriate region and therefore the desired hairpin may not be generated.

This base-pairing at the 3′ terminus of the hairpin may be achieved either by (i) precise termination through the use of a PolIII polymerase, mitochondrial RNA polymerase, chloroplast RNA polymerase, bacterial RNA polymerase, phage RNA polymerase, or viral RNA polymerase or through the use of template DNA that is linearized (i.e., restriction digested or PCR amplified) precisely at the desired end sequences. Linear supercoiled templates may also be prepared through the use of phage N15 in which desired sequences are incorporated into the phage genome and propagated by helper phage. Alternatively, RNA polII transcripts that have been polyadenylated can be used since a stretch of poly U in the template strand allows a fraction of the mRNA molecules to assume a hairpin structure in which the terminal A sequences are flush base-paired with the template polyU (FIG. 7A). Oligodeoxyribonucleotides, modified oligonucleotides that allow RNase H cleavage of RNA sequence (e.g., phosphonates, PNAs (peptide nucleic acids), phosphorothioates, or imidophosphate), or RNA in which the hydroxy group at the 2′-position of the ribose sugar is replaced by a modifier group (e.g., a halo, sulfhydryl, azido, amino, monosubstituted amino, or disubstituted amino group) that allow RNase H mediated cleavage such as cleavage that occurs in the antisense mechanism can be used (FIG. 7B). Ribozymes (e.g., hairpin, hammerhead, self-splicing such as tetrahymena or phage T4 td intron, or HDV or RNase P mediated ribozymes) can be used to cleave the RNA template in trans (FIG. 7C) by expression from a separate plasmid or separate cistron. Alternatively, any of the ribozymes can be used to cleave the RNA template in cis. For example, a ribozyme can be placed within the hairpin loop to cleave 3′ sequences of the RNA template (FIG. 7D). Alternatively, a ribozyme can be placed in the 3′ portion of the RNA template to cleave a 5′ position, relative to the ribozyme, on the RNA template (FIG. 7E). The rate and the specificity of this reaction can be further enhanced, if desired, through the use of anchor sequences that base pair with sequences in the transcript preferably in the hairpin loop sequences (FIG. 7F). In other methods, tRNA sequences are placed at the 3′ end to allow RNase P cleavage (FIG. 7G). Moreover, trans-splicing ribozymes that snatch sequences from another specific mRNA molecules can be used to generate a chimeric RNA molecule with the desired 3′ end (FIG. 7H (i) and (ii)).

The aforementioned methods are illustrated in FIGS. 7A-7H. Other embodiments of these methods can be performed by one skilled in the art using standard methods. For example, FIGS. 7C-7F illustrate methods including a hammerhead ribozyme, and FIG. 7H (i) and (ii) illustrates methods including a self-slicing tetrahymena ribozyme; however, any other ribozyme can be used in these methods.

If desired, an anchor can be used in any of the constructs to increase local concentrations and/or causes unfolding of RNA molecules due to imposed base-pairing by the anchor sequences, potentially increasing exposure of the target sequence. Thus, anchored ribozymes may be used whenever cleavage is slow or inefficient, independent of the ribozyme being in trans or in cis with respect to the target. The ribozyme and anchor are in cis with each other. See, e.g., the teaching of Pachuk et al., U.S. Pat. No. 6,080,851, concerning ribozymes with linked anchor sequences.

The following exemplary references describe the design and use of ribozymes: HDV ribozyme, Fiola & Perreault, J. Biol. Chem. (2002) 277: 26508-16; Hammerhead ribozyme, Seghan et al., J. Biol. Chem. (2002) 277: 25957-25962; Hairpin ribozyme, Durel et al., RNA (2002) 8: 336-344; HDV ribozyme, Acta. Biochim. (Poland) (2001) 48: 409; Layered viral replication system, Dubensky et. al., U.S. Pat. No. 5,814,482; RNase P, Yuan et. al., U.S. Pat. No. 5,869,248; Hairpin ribozyme, Chowira & McSwiggen, U.S. Pat. No. 6,022,962; Ribozyme, Urdea Michael, U.S. Pat. No. 5,631,148; Trans-splicing ribozymes, Haseloff and Goodman, U.S. Pat. No. 6,015,794; Trans-splicing ribozymes, U.S. Pat. No. 5,667,969; modifications of the nucleic acids to allow RNase H and ribozyme cleavage, Benseler, WO9207065; RNA polymerase III-based expression of therapeutic RNA molecules, Thompson, U.S. Pat. No. 6,146,886; RNase P, Sha et al., U.S. Pat. No. 6,057,153; ribonuclease P, Yan et al., U.S. Pat. No. 5,869,248; RNA ribozyme polymerases, dephosphorylases, and restriction endoribonucleases, Cech et al., U.S. Pat. No. 6,180,399, and ribozymes with linked anchor sequences, Pachuk et al., U.S. Pat. No. 6,080,851.

Exemplary methods are described below.

Ribozyme/Self-Splicing Intron Mediated RNA Cleavage

Examples of methods to process RNA molecules at discrete sites include ribozyme-mediated cleavage of RNA at a particular site (FIGS. 5A and 5B). The ribozyme can be encoded within the target RNA molecule as depicted and act in cis to cleave the RNA at the target site (FIG. 5A) or can be expressed from a separate vector or cistron and therefore act in trans to cleave the RNA at the target site. Examples of types of ribozymes that can be used are hammerhead and self-splicing introns, such as Group 1 introns from Tetrahymena (available from ATCC). Both of these ribozymes can be used in trans or in cis. The released RNA containing the ribozyme is catalytically active and can cleave unprocessed target RNA molecules. The unprocessed target RNA molecules therefore have the potential to be cleaved by a ribozyme in trans or by the ribozyme that is part of the target molecule. A ribozyme or a target molecule can also cleave a second target RNA in trans. Techniques for the design of hammerhead ribozymes useful in the methods of the invention can be found in for example, Pachuk et al., Nucleic Acids Research, 22: 301-307, 1994. The design of self-splicing and trans-splicing Group I introns is taught by, e.g., Sullenger and Cech, Nature, pp. 619-622, 1994. After ribozyme-mediated cleavage of the RNA molecule at a particular site yields a partial dsRNA hairpin with a 3′ terminus base-paired to the desired upstream region, the 3′ terminus can be self extended as taught herein, using an RNA-dependent RNA polymerase.

RNase H Mediated Cleavage

Another method that can be used to process RNA at a particular site is to use RNase H-mediated cleavage. In this method, a DNA oligonucleotide that is complementary to the region desired to be cleaved is hybridized to the RNA. To yield the desired partial hairpin RNA, a DNA oligonucleotide is hybridized to a sequence of a region 2 (BPS-2) of the RNA molecule (see FIG. 7B). The DNA oligonucleotide will be at least about 7 nucleotides in length, but can be much longer, with no upper limit per se, depending on the length of the RNA to be cleaved. The DNA/RNA hybridization site is selected so that there are at least 5 to 15 nucleotides within region 2 upstream of the hybridization site. These nucleotides will remain as the 3′ terminus of the molecule after RNase cleavage and will be available to base-pair with complementary nucleotides in an upstream region 1. For in vitro cleavage, RNase H (New England Biolabs, Beverly Mass.) is incubated with the DNA/RNA hybrid according to the manufacturer's directions. The RNA is cleaved at multiple sites within the hybrid region, thereby resulting in a 3′ end that is complementary to the template (FIGS. 4A-4B). This reaction can also be performed intracellularly because RNase H is expressed within the cell as an endogenous enzyme. For this intracellular reaction, the DNA oligonucleotide can be co-delivered with the dsRNA or the dsRNA expression construct using standard methods, such as delivery methods for in vivo administration of antisense molecules to cell cultures or animals. After RNase mediated cleavage yields a partial dsRNA hairpin with a 3′ terminus base-paired to the desired upstream region, the 3′ terminus can be self extended as taught herein, using an RNA-dependent RNA polymerase.

A Partial Hairpin with Predefined Terminus that Base-Pairs with Another Region of the Molecule

An alternative strategy uses a linear DNA template in which one end encodes the desired 3′ end of the partial hairpin. The linear template can be used in vitro or transfected into cells/animals for in vivo use. The discrete end can be generated by a number of means including PCR in which the end is defined by the sequence of the PCR primer or by restriction analysis.

A Partial Hairpin with a Termination Signal that Results in a 3′ Terminus that Base-Pairs with Another Region of the Molecule

Another approach for generating a partial hairpin capable of being extended is to use transcription termination signals to terminate transcription such that the 3′ terminal nucleotides are complementary to another region in the hairpin. In this method, RNA polIII is used to generate a partial RNA hairpin in which the 3′ end is generated via an RNA PolIII transcription termination signal (e.g., a string of at least 4-5 T nucleotides, desirably at least 5 to 15 T nucleotides). As transcription terminates in this cluster of T's, the 3′ end is comprised of T nucleotides. The complementary A nucleotides are built into the construct at the indicated site in the template, resulting in a partial hairpin that is completely base-paired with the template at the 3′ end.

In the following example, an RNA PolIII upstream promoter element is used to direct the nuclear synthesis of a partial hairpin. RNA polIII transcription is terminated in a run of at least 4 to 5, desirably at least 5 to 15 T residues (FIG. 6A). A similar number of complementary A residues are included at a selected upstream position to allow base-pairing of the terminal T residues with the complementary A residues. The 3′ terminal nucleotides must be base-paired with an upstream region which serves as the template in order to permit extension of the 3′ end.

For generation of the reagents of the present invention, a mammalian vector is created that generates a short terminal hairpin to facilitate reverse primed replication of any cloned target cDNA into dsRNA by an endogenous or exogenous RNA-dependent RNA polymerase (RdRp). Any RNA PolIII promoter that is classified as an upstream or internal promoter (e.g., an internal promoter reported by Donald Brown of the Carnegie Institute or the 5sRNA or tRNA promoter) can be used, such as a vector that utilizes the upstream RNA polymerase III type I U6 promoter to express large amounts of dsRNA in the nucleus. Usefulness of this construct can be demonstrated by the expression of dsRNA derived from a short 200 base-paired segment of secreted alkaline phosphatase (SEAP).

The vector component of this invention termed pU6rp contains the 272 bp U6 promoter, a small multiple cloning site (MCS), RNA polIII termination sites, and a 30 bp hairpin (FIG. 6). The vector component of this invention termed pU6rp U6 reverse-primed contains the 272 base-pair U6 promoter, a small multiple cloning site (MCS), RNA polIII termination sites consisting of a consecutive string of at least 4 or 5 T's, and an artificial 30 base-pair hairpin (FIG. 6A). Following the insertion of a cDNA of interest, transfection of these constructs into mammalian cells produces nuclear RNA polIII transcripts that span the cloned cDNA and continue through a 3′ terminal hairpin/polIII termination region, RNA polIII terminates at the first stretch of 4 or 5 consecutive T's generally after the second T and expose the hairpin region. This vector-encoded hairpin provides a suitable 3′ hydroxyl substrate for the RdRp-mediated extension that generates the desired target dsRNA (FIG. 6B).

To generate pU6rp for this method, the U6 promoter is obtained by PCR amplification using the plasmid pTZU6+1 as template (Lee, et al., Nature Biotechnology, 2002, pp. 500-505). The vector cloning sites and hairpin are included in the PCR primers used for this amplification. The PCR product contains the 272 base-pair U6 promoter and the sequence between the BamHI and SalI sites of pTZU6+1. The MCS for cDNA insertion consists of the restriction enzymes SalI, SmaI, and BglII. The 5′ forward primer includes a terminal EcoRI site, and the 3′ primer includes a HindIII site to facilitate cloning of the PCR product into EcoRI/HindIII sites of pUC19 to obtain the final vector, pU6rp. The sequence of the 5′ primer is 5′CCGGAATTCGGATCCAAGGTCGGGCAGG, and the sequence of the 3′ primer is

5′GCGAAGCTTAAAAATCTAGAAAAAGGGTGTGGTGCTAGCACCACACCC
TTTAGATCTCCCGGGTCGACCGGTGTTTCGTCC.

To generate the SEAP portion of the construct, a 200 base-pair fragment is PCR amplified from pSEAP2 (sequences 1008-1207 of Genbank accession number U89938) and is cloned into the SmaI site of pU6rp. Isolates of both orientations are selected and termed pU6rp-SEAPf and pU6rp-SEAPr. The sequence of the 5′ primer is 5′ ACGGGAAGAATCTGGTGCAG 3′, and the sequence of the 3′ primer is 5′ GGCAGCCTCTGTCATCTCCA 3′. This vector allows the excision of the second half of the terminal hairpin by simple digestion and religation reactions to provide the corresponding control plasmids that lack the ability to reverse prime the cloned cDNA. To generate these control vectors, the pU6rp-SEAP constructs are digested with XbaI+NheI and religated to generate the plasmids pU6-SEAPf and pU6-SEAPr (FIG. 6B).

Demonstration of the self-extension of partial hairpins can be performed by mammalian cell transfection using standard methods as described below. The transfected cells are cells that either stably or transiently express SEAP. In this particular example, the cells are RD cells transiently expressing SEAP. The positive control is the pair of pU6-SEAP vectors (pU6-SEAPf and pU6-SEAPr) that together generate both sense and antisense strands of SEAP to form intracellular dsRNA. The negative controls are either of these pU6-SEAP plasmids transfected alone because they should be incapable of generating dsRNA without a terminal hairpin to prime RdRp second strand synthesis. The other plasmids are the pU6rp-SEAP constructs expected to generate dsRNA via this RdRp mechanism. The expected results are that both the positive control plasmids (the pair of pU6-SEAP vectors) and the pU6rp-SEAP construct inhibit SEAP expression whereas the negative control plasmids have no effect.

The formation of hairpins by self-extension of partial hairpins has many advantages. The single cDNA cloning step and stability in bacteria facilitates dsRNA-expressing cDNA library generation. In contrast, traditional synthesis of hairpins often requires tedious cloning procedures and may involve constructs that are not stable in bacteria. The present methods should also result in more efficient creation of dsRNA because any folding problems are minimized by RdRp read through. Accordingly, the partial hairpin constructs of the invention offer significant advantages in generating dsRNA libraries for functional genomics applications as described in more detail elsewhere herein, and can advantageously be provided as kits which include a source of RdRp. E.g., standard cloning vector(s) with two MCSs flanking A/Loop/B sequences as shown in FIG. 1E can advantageously be provided in a commercial kit useful, e.g., for functional genomics applications. The kit may also provide a source of RdRp. Bi- or multicistronic constructs comprising one or more of such promoter/MCS/A/Loop/B/MCS units, together with other expression units, e.g., expressing a RNA dependent RNA polymerase (RdRp; see, e.g., FIG. 1E(ii)), can be advantageously utilized, e.g., for expression and extension of “partial” hairpins, as described herein.

### Example 7

### Multiple-Epitope Double Stranded RNA Approach

Significant advantages can be obtained by using dsRNA with segments or epitopes derived from (1) sequences representing multiple genes of a single organism; (2) sequences representing one or more genes from a variety of different organisms; and/or (3) sequences representing different regions of a particular gene. Using this approach, a singular species of dsRNA can be engineered to simultaneously target many different genes and/or many organisms, e.g, pathogens, including viral and/or bacterial pathogenic agents. Alternatively, the singular species of dsRNA can be used to target a subset of genes or organisms on one occasion and the same or a second subset on another occasion. The dsRNA can be, e.g., a duplex or a hairpin and can be encoded by a DNA or RNA vector. The RNA can be expressed intracellularly in the host or made in vitro and then subsequently administered to the host, as described herein. This “multiple epitope,” at least partially double-stranded RNA molecules can assume a variety of structural variations, including the partial hairpins and forced hairpins described in detail herein, and further, as described, for example, in Pachuk and Satishchandran, WO 00/63364, the teaching of which is incorporated herein by reference. The host cell can be a cell in vitro or in vivo, such as a cell in a tissue or an organism (e.g., a cell in a plant or animal, including invertebrate and vertebrate animals, or mammal such as a human or commercially important species such as a bovine, equine, canine, feline, or avian).

One particularly desirable multiple epitope approach involves targeting both a selected target gene(s) and the promoter(s) which drives transcription of that gene, resulting in a combination of post-transcriptional and transcriptional gene silencing (PTGS and TGS). This combination of gene silencing has the advantage of achieving a rapid gene silencing response that is maintained for a long duration or permanently in the host.

Advantages of a Multiple Epitope Double Stranded RNA Approach

Because a singular species of dsRNA can simultaneously target and silence many genes (e.g., genes from multiple pathogens or genes associated with multiple diseases), a multiple epitope dsRNA can be used for many different indications in the same subject or used for a subset of indications in one subject and another subset of indications in another subject. Due to the growing concern about terrorism and the potential threat of biological warfare, a multiple epitope dsRNA is useful as a non-toxic agent that can provide protection against a number of different organisms for an extended period of time, if not permanently. Particularly promising is a DNA construct capable of intracellular expression in a host of an at least partially double-stranded RNA comprising dsRNA sequences exhibiting homology with one or more genes of a number of different potential pathogenic organisms, including viruses such as smallpox, Ebola, Marburg, HIV-1, HIV-2, Dengue, Yellow fever, or influenza. The dsRNA can also include sequences for host cellular receptors for viral and/or bacterial genes and/or viral and/or bacterial toxins (e.g., cellular receptors for toxins from Anthrax, Diphtheria, or Botulinum toxin). For such applications, the ability to express long dsRNA molecules (e.g., dsRNA molecules with sequences from multiple genes) without invoking the dsRNA stress response is highly desirable. For example, by using a series of sequences, each, e.g., as short as 19-21 nucleotides, preferably 100 to 600 nucleotides, or easily up to 1, 2, 3, 4, 5, or more kilobases such that the total length of such sequences is within the maximum capacity of the selected plasmid (e.g., 20 kilobases in length), a single such pharmaceutical composition can provide protection against a large number of pathogens and/or toxins at a relatively low cost and low toxicity. Importantly, this same approach can be used to provide protection against biological warfare agents that affect important food crops such as wheat or rice or commercially important animals such as cattle, sheep, goats, pigs, poultry, or fish.

Examples of viral pathogens that may be suitable targets for application of the multiple epitope dsRNA approach include HIV-1, HIV-2, smallpox, vaccinia, encephalitic viruses (e.g., West Nile, Japanese encephalitis, and equine encephalitis), Dengue, Yellow fever, Ebola, Marburg, measles, polio, influenza, hepatitis viruses (e.g., Hepatitis A, B, and C), Herpes simplex 1 and 2, EBV, HCMV, as well as species of the Retrovirus, Herpesvirus, Hepadnavirus, Poxvirus, Parvovirus, Papillomavirus, and Papovavirus families. Some of the more desirable viral infection to treat or prevent with this method include, without limitation, infections caused by HIV, HBV, HSV, CMV, HPV, HTLV, or EBV. Particularly suitable for such treatment are DNA viruses or viruses that have an intermediary DNA stage. The target gene(s) or fragment thereof is desirably a virus polynucleotide sequence that is necessary for replication and/or pathogenesis of the virus in an infected mammalian cell. Among such target polynucleotide sequences are protein-encoding sequences for proteins necessary for the propagation of the virus, e.g., the HIV gag, env, and pol genes as well as necessary regulatory genes; the HPV6 L1 and E2 genes; the HPV11 L1 and E2 genes; the HPV16 E6 and E7 genes; the HPV18 E6 and E7 genes; the HBV surface antigen, core antigen, and reverse transcriptase; the HSV gD gene; the HSVvp16 gene; the HSVgC, gH, gL, and gB genes; the HSV ICP0, ICP4 and ICP6 genes; Varicella zoster gB, gC, and gH genes; the BCR-abl chromosomal sequences, and non-coding viral polynucleotide sequences which provide regulatory functions necessary for transfer of the infection from cell to cell, e.g., HIV LTR and other viral promoter sequences, such as HSV vp16 promoter, HSVICP0 promoter; HSV-ICP4, ICP6 and gD promoters, the HSV surface antigen promoter; or the HBV pre-genomic sequence. Other exemplary targets are described in Pachuk and Satishchandran, WO 00/63364, and in U.S. Pat. No. 6,506,559, Fire et al., the teaching of which is hereby incorporated by reference,

The use of multiple epitopes derived from one or more genes from multiple strains and/or variants of a highly variable or rapidly mutating pathogen such as HIV, HCV, or influenza can also be very advantageous. For example, a singular dsRNA species that recognizes and targets multiple strains and/or variants of the influenza virus can be used as a universal treatment or vaccine for the various strains/variants of influenza.

The ability to silence multiple genes of a particular pathogen such as HIV prevents the selection of, in this case, HIV “escape mutants.” In contrast, typical small molecule treatment or vaccine therapy that only targets one gene or protein results in the selection of pathogens that have sustained mutations in the target gene or protein and the pathogen thus becomes resistant to the therapy. By simultaneously targeting a number of genes of the pathogen and/or extensive regions of the pathogen using the multiple epitope approach of the present invention, the emergence of such “escape mutants” is effectively precluded.

This multiple epitope approach is also particularly suitable for the treatment of cancers that result from the over-expression of more than one gene product. Such gene products, by definition, are needed to maintain the cancerous state of the tumor cell or tumor. One singular dsRNA species can act to target the multiple RNA molecules encoding these different gene products or a subset of these gene-products. Thus, one pharmaceutically active dsRNA silences the multiple components that have led to the cancerous phenotype. Examples of human cancers include cervical, ovarian, lung, colon, leukemias, lymphomas, breast, prostate, testicular, uterine, melanoma, liver, head and neck, malignant brain, and stomach cancer. Oncogenes are suitable targets for the dsRNA of the invention (including, e.g., ABL1, BCL1, BCL2, BCL6, CBFA2, CBL, CSF1R, ERBA, ERBB, EBRB2, FGR, FOS, FYN, HCR, HRAS, JUN, KRAS, LCK, LYN, MDM2, MLL, MYB, MYC, MYCL1, MYCN, NRAS, and RAS). Tumor suppressor genes, (e.g., APC, BCRA1, BCRA2, MADH4, MCC, NF1, NF2, RB1, and TP530), enzymes (e.g., kinases), cancer-associated viral targets (e.g., HPV E6/E7 virus-induced cervical carcinoma, HTLV-induced cancer, and EBV-induced cancers such as Burkitt's Lymphoma) can also be targeted. In the latter instance, a composition can be administered in which the target polynucleotide is a coding sequence or fragment thereof, or a non-expressed regulatory sequence for an antigen or sequence that is required for the maintenance of the tumor in the host animal. Exemplary targets include HPV16 E6 and E7 and HPV 18 E6 and E7 sequences. Others may be readily selected by one of skill in the art. In developing multiple epitope constructs directed toward a cancer-related polynucleotide sequence with a single point mutation as compared to the normal sequence, it may be advantageous to string together a series of overlapping 21-mers (19-23mers), each of which contains the mutation that distinguishes the abnormal sequence.

Pharmaceutical Compositions

A pharmaceutical composition can be prepared as described herein comprising a DNA plasmid construct expressing, under the control of a bacteriophage T7 promoter, a dsRNA substantially homologous to, e.g., one or more genes from the smallpox virus and human cell receptor sequences for the Anthrax toxin. The T7 RNA polymerase can be co-delivered and expressed from the same or another plasmid under the control of a suitable promoter e.g., hCMV, simian CMV, or SV40. In some embodiments, the same or another construct expresses the target gene (e.g., a target smallpox gene) contemporaneously with the dsRNA homologous to the target smallpox gene. The pharmaceutical composition is prepared in a pharmaceutical vehicle suitable for the particular route of administration. For IM, SC, IV, intradermal, intrathecal or other parenteral routes of administration, a sterile, non-toxic, pyrogen-free aqueous solution such as Sterile Water for Injection, and, optionally, various concentrations of salts, e.g., NaCl, and/or dextrose, (e.g., Sodium Chloride Injection, Ringer's Injection, Dextrose Injection, Dextrose and Sodium Chloride Injection, and Lactated Ringer's Injection) is commonly used. Optionally, other pharmaceutically appropriate additives, preservatives, or buffering agents known to those in the art of pharmaceutics are also used. If provided in a single dose vial for injection, the dose will vary as determined by those of skill in the art of pharmacology, but may typically contain between 5 mcg to 500 mcg of the active construct. If deemed necessary, significantly larger doses may be administered without toxicity, e.g., up to 5-10 mg.

The DNA and/or RNA constructs of the invention may be administered to the host cell/tissue/organism as “naked” DNA, RNA, or DNA/RNA, formulated in a pharmaceutical vehicle without any transfection promoting agent. More efficient delivery may be achieved as known to those of skill in the art of DNA and RNA delivery, using e.g., such polynucleotide transfection facilitating agents known to those of skill in the art of RNA and/or DNA delivery. The following are exemplary agents: cationic amphiphiles including local anesthetics such as bupivacaine, cationic lipids, liposomes or lipidic particles, polycations such as polylysine, branched, three-dimensional polycations such as dendrimers, carbohydrates, detergents, or surfactants, including benzylammonium surfactants such as benzylkonium chloride. Non-exclusive examples of such facilitating agents or co-agents useful in this invention are described in U.S. Pat. Nos. 5,593,972; 5,703,055; 5,739,118; 5,837,533; 5,962,482; 6,127,170; and 6,379,965, as well as International Patent Application Nos. PCT/US03/14288, filed May 6, 2003 (multifunctional molecular complexes and oil/water cationic amphiphile emulsions), and PCT/US98/22841; the teaching of which is hereby incorporated by reference. U.S. Pat. Nos. 5,824,538; 5,643,771; and 5,877,159 (incorporated herein by reference) teach delivery of a composition other than a polynucleotide composition, e.g., a transfected donor cell or a bacterium containing the dsRNA-encoding compositions of the invention.

### Example 8

### Exemplary Methods for Enhancing dsRNA-mediated Gene Silencing

Mammalian Origin of Replication

An origin of replication enables the DNA plasmid to be replicated upon nuclear localization and thus enhances gene silencing. The advantage is that more plasmid is available for nuclear transcription and therefore more RNA effector molecules are made (e.g., more hairpins and/or more duplexes). Many origins are species-specific and work in several mammalian species but not in all species. For example, the SV40 T origin of replication (e.g., from plasmid pDsRed1-Mito from Clontech; U.S. Pat. No. 5,624,820) is functional in mice but not in humans. This origin can thus be used for vectors that are used or studied in mice. Other origins that can be used for human applications, such as the EBNA origin (e.g., plasmids pSES.Tk and pSES.B from Qiagen). DNA vectors containing these elements are commercially available, and the DNA segment encoding the origin can be obtained using standard methods by isolating the restriction fragment containing the origin or by PCR amplifying the origin. The restriction maps and sequences of these vectors are available publicly and enable one skilled in the art to amplify these sequences or isolate the appropriate restriction fragment. These vectors replicate in the nuclei of cells that express the appropriate accessory factors such as SV40 TAg and EBNA. The expression of these factors is easily accomplished because some of the commercially available vectors (e.g., pSES.Tk and pSES.B from Qiagen) that contain the corresponding origin of replication also express either SV40 Tag or the EBNA. These DNA molecules containing the origin of replication can be easily cloned into a vector of interest (e.g., a vector expressing a dsRNA such as a hairpin or duplex) by one skilled in the art. These vectors are then co-transfected, injected, or administered with a vector expressing EBNA or Tag to enable replication of the plasmid bearing the EBNA or Tag origin of replication, respectively. Alternatively, the genes encoding EBNA or Tag are cloned into any another expression vector designed to work in the cells, animal, or organism of interest using standard methods. The genes encoding EBNA and Tag can also be cloned into the same vector bearing the origin of replication. Suitable origins of replication are not limited to Tag and EBNA; for example, Replicor in Montreal has identified a 36 base-pair mammalian origin consensus sequence that permits the DNA sequence to which it is attached to replicate (as reviewed in BioWorld Today, Aug. 16, 1999, Volume 10, No. 157). This sequence does not need the co-expression of auxiliary sequences to enable replication.

Replication of dsRNA

Alternatively or additionally, the transcribed dsRNA molecules can be amplified. RNA can be replicated by a variety of RNA-dependent RNA polymerases provided the appropriate replication signals are encoded at the 3′ ends of the RNA molecules. Examples are provided in the following references: Driver et al., Ann NY Acad Sci 1995, 261-264, and Dubensky et al, J Virol, 1996, 508-519. Other exemplary RNA dependent-RNA polymerases (e.g., viral, plant, invertebrate, or vertebrate such as mammalian or human polymerases) are listed in Table 1. Additional suitable RNA dependent-RNA polymerases include alphaviral polymerases, Semliki Forest viral polymerases, and polymerases from mammalian viruses, invertebrates, and plants. The RNA molecules that are replicated by cytoplasmic RNA polymerases can be transcribed in the nucleus followed by cytoplasmic localization, or they can be transcribed in the cytoplasm.

### Example 9

### Exemplary Methods for the Administration of dsRNA

The short dsRNA molecules and/or long dsRNA molecules of the invention may be delivered as “naked” polynucleotides, by injection, electroporation, and any polynucleotide delivery method known to those of skill in the field of RNA and DNA. For example, in vitro synthesized dsRNA may be directly added to a cell culture medium. Uptake of dsRNA is also facilitated by electroporation using those conditions required for DNA uptake by the desired cell type. RNA uptake is also mediated by lipofection using any of a variety of commercially available and proprietary cationic lipids, DEAE-dextran-mediated transfection, microinjection, protoplast fusion, calcium phosphate precipitation, viral or retroviral delivery, local anesthetic RNA complex, or biolistic transformation.

Alternatively, the RNA molecules may by delivered by an agent (e.g., a double stranded DNA molecule) that generates an at least partially double stranded molecule in cell culture, in a tissue, or in vivo in a vertebrate or mammal. The DNA molecule provides the nucleotide sequence which is transcribed within the cell to become an at least partially double stranded RNA. These compositions desirable contain one or more optional polynucleotide delivery agents or co-agents, such as a cationic amphiphile local anesthetic such as bupivacaine, a peptide, cationic lipid, a liposome or lipidic particle, a polycation such as polylysine, a branched, three-dimensional polycation such as a dendrimer, a carbohydrate, a cationic amphiphile, a detergent, a benzylammonium surfactant, one or more multifunctional cationic polyamine-cholesterol agents disclosed in U.S. Pat. No. 5,837,533, and U.S. Pat. No. 5,837,533; U.S. Pat. No. 6,127,170; U.S. Pat. No. 5,962,428, U.S. Pat. No. 6,197,755, WO 96/10038, published Apr. 4, 1996, WO 94/16737, published Aug. 8, 1994, and PCT/US03/14288, filed May 6, 2003 (multifunctional molecular complexes and oil/water cationic amphiphile emulsions), the teaching of which are hereby incorporated by reference.

For administration of dsRNA as taught in U.S. Ser. No. 60/375,636 filed Apr. 26, 2002 and U.S. Ser. No. 10/425,006 filed Apr. 28, 2003 “Methods for Silencing Genes Without Inducing Toxicity”, C. Pachuk, the teaching of which is incorporated herein by reference, (e.g., a short dsRNA to inhibit toxicity or a short or long dsRNA to silence a gene) to a cell or cell culture, typically between 50 ng and 5 ug, such as between 50 ng and 500 ng or between 500 ng and 5 ug dsRNA is used per one million cells. For administration of a vector encoding dsRNA (e.g., a short dsRNA to inhibit toxicity or a short or long dsRNA to silence a gene) to a cell or cell culture, typically between 10 ng and 2.5 ug, such as between 10 ng and 500 ng or between 500 ng and 2.5 ug dsRNA is used per one million cells. Other doses, such as even higher doses may also be used.

For administration of dsRNA (e.g., a short dsRNA to inhibit toxicity or a short or long dsRNA to silence a gene) to an animal, typically between 10 mg to 100 mg, 1 mg to 10 mg, 500 ug to 1 mg, or 5 ug to 500 ug dsRNA is administered to a 90-100 pound person or animal (in order of increasing preference.) For administration of a vector encoding dsRNA (e.g., a short dsRNA to inhibit toxicity or a short or long dsRNA to silence a gene) to an animal, typically between 100 mg to 300 mg, 10 mg to 100 mg, 1 mg to 10 mg, 500 ug to 1 mg, or 50 ug to 500 ug dsRNA is administered to a 90-100 pound person (in order of increasing preference. The dose may be adjusted based on the weight of the animal. In some embodiments, about 1 to 10 mg/kg or about 2 to 2.5 mg/kg is administered. Other doses may also be used.

For administration in an intact animal, typically between 10 ng and 50 ug, between 50 ng and 100 ng, or between 100 ng and 5 ug of dsRNA or DNA encoding a dsRNA is used. In desirable embodiments, approximately 10 ug of a DNA or 5 ug of dsRNA is administered to the animal. With respect to the methods of the invention, it is not intended that the administration of dsRNA or DNA encoding dsRNA to cells or animals be limited to a particular mode of administration, dosage, or frequency of dosing; the present invention contemplates all modes of administration sufficient to provide a dose adequate to inhibit gene expression, prevent a disease, or treat a disease. The doses may be adjusted based on the weight of the animal, the effect to be achieved, and the route of administration, as can be determined without undue experimentation by those of skill in the art of pharmacology.

If desired, short dsRNA is delivered before, during, or after the delivery of dsRNA (e.g., a longer dsRNA) that might otherwise be expected to induce cytotoxicity. Modulation of cell function, gene expression, or polypeptide biological activity may then be assessed in the cells or animals.

### Example 10

### Exemplary Methods for Using the dsRNAs of the Invention in dsRNA-Mediated Gene Silencing to Determine or Validate the Function of a Gene

The dsRNAs of the invention, including the dsRNA partial and/or forced hairpin structures, and the dsRNA expression constructs encoding such partial and/or forced hairpin structures, and kits providing such dsRNAs and/or dsRNA expression constructs, including such kits which provide a source of RdRp, may be advantageously utilized in various functional genomics applications as described in more detail below.

DsRNA-mediated gene silencing can be used as a tool to identify and validate specific unknown genes involved in cell function, gene expression, and polypeptide biological activity. Since novel genes are likely to be identified through such methods, PTGS is developed for use in validation and to identify novel targets for use in therapies for diseases, for example, cancer, neurological disorders, obesity, leukemia, lymphomas, and other disorders of the blood or immune system.

The dsRNAs and dsRNA expression constructs of the invention can be advantageously used in the methods taught in U.S. Published Application 2002/0132257 and European Published Application 1229134, “Use of post-transcriptional gene silencing for identifying nucleic acid sequences that modulate the function of a cell”, the teaching of which is hereby incorporated by reference. The methods involve the use of double stranded RNA expression libraries, double stranded RNA molecules, and post-transcriptional gene silencing techniques. Particularly preferred for utilization of the dsRNAs and dsRNA expression constructs of the invention are such methods wherein cDNA libraries are utilized to obtain a single integration per cell and expression of a single dsRNA per cell. In some embodiments, once a stable integrant containing five or fewer, and desirably no episomal expression vectors, transcription is induced, allowing dsRNA to be expressed in the cells. This method ensures that, if desired, only one species or not more than about five species of dsRNA is expressed per cell, as opposed to other methods that express hundreds to thousands of double stranded species.

These methods provide a highly efficient means for identifying nucleic acid sequences which, e.g., confer or are associated with a detectable phenotype, e.g., nucleic acid sequences that modulate the function of a cell, the expression of a gene in a cell, or the biological activity of a target polypeptide in a cell. A detectable phenotype may include, for example, any outward physical manifestation, such as molecules, macromolecules, structures, metabolism, energy utilization, tissues, organs, reflexes, and behaviors, as well as anything that is part of the detectable structure, function, or behavior of a cell, tissue, or living organism. Such methods are useful in a variety of valuable applications including high throughput screening methods for identifying and assigning functions to unknown nucleic acid sequences, as well as methods for assigning function to known nucleic acid sequences. A particularly advantageous aspect of such methods is that the transformation of vertebrate cells, including mammalian cells, and the formation of double stranded RNA are carried out under conditions that inhibit or prevent an interferon response or a double stranded RNA stress response.

The dsRNAs and dsRNA expression constructs of the invention can be advantageously used in the following methods which use site-specific recombination to obtain single integrants (or desirably no more than five) of dsRNA expression cassettes at the same locus of all cells in the target cell line, allowing stable and uniform expression of the dsRNA in all of the integrants. A dsRNA expression library derived from various cell lines is used to create a representative library of stably integrated cells, each cell within the target cell line containing a single integrant. Cre/lox, Lambda-Cro repressor, and Flp recombinase systems or retroviruses may be used to generate these singular integrants of dsRNA expression cassettes in the target cell line. A desirable vector may comprise two convergent T7 promoters, two convergent SP6 promoters, or one convergent T7 promoter and one convergent SP6 promoter, a selectable marker, and/or a loxP site. (Satoh et al., J. Virol. 74:10631-10638, 2000; Trinh et al., J. Immunol. Methods 244:185-193, 2000; Serov et al., An. Acad. Bras. Cienc. 72:389-398, 2000; Grez et al, Stem Cells. 16:235-243, 1998; Habu et al., Nucleic Acids Symp. Ser. 2:295-296, 1999; Haren et al., Annu. Rev. Microbiol. 53:245-281, 1999; Baer et al., Biochemistry 39:7041-7049, 2000; Follenzi et al., Nat. Genet. 25:217-222, 2000; Hindmarsh et al., Microbiol. Mol. Biol. Rev. 63:836-843, 1999; Darquet et al., Gene Ther. 6:209-218, 1999; Darquet et al., Gene Ther. 6:209-218, 1999; Yu et al, Gene 223:77-81, 1998; Darquet et al., Gene Ther. 4:1341-1349, 1997; and Koch et al., Gene 249:135-144, 2000). These systems are used singularly to generate singular insertion clones, and also in combination.

The following exemplary sequence specific integrative systems use short target sequences that allow targeted recombination to be achieved using specific proteins: FLP recombinase, bacteriophage Lambda integrase, HIV integrase, and pilin recombinase of Salmonella (Seng et al. Construction of a Flp “exchange cassette” contained vector and gene targeting in mouse ES cell; A book chapter PUBMED entry 11797223-Sheng Wu Gong Cheng Xue Bao. 2001 September; 17(5):566-9; Liu et al., Nat Genet. 2001 Jan. 1; 30(1):66-72; Awatramani et al., Nat Genet. 2001 November; 29(3):257-9; Heidmann and Lehner, Dev Genes Evol. 2001 September; 211(8-9):458-65; Schaft et al., Genesis 2001 September; 31(1): 6-10; Van Duyne, Annu Rev Biophys Biomol Struct. 2001; 30:87-104; Lorbach et al., J Mol Biol. 2000 Mar. 10; 296(5):1175-81; Darquet et al., Gene Ther. 1999 February; 6(2):209-18; Bushman and Miller, J Virol. 1997 January; 71(1):458-64; Fulks et al., J. Bacteriol. 1990 January; 172(1):310-6). A singular integrant is produced by randomly inserting the specific sequence (e.g., loxP in the cre recombinase system) and selecting or identifying the cell that contains a singular integrant that supports maximal expression. For example, integrants that show maximal expression following random integration can be identified through the use of reporter gene sequences associated with the integrated sequence. The cell can be used to specifically insert the expression cassette into the site that contains the target sequence using the specific recombinase, and possibly also remove the expression cassette that was originally placed to identify the maximally expressing chromosomal location.

A skilled artisan can also produce singular integrants using retroviral vectors, which integrate randomly and singularly into the eukaryotic genome. In particular, singular integrants can be produced by inserting retroviral vectors that have been engineered to contain the desired expression cassette into a naive cell and selecting for the chromosomal location that results in maximal expression (Michael et al., EMBO Journal, vol 20: pages 2224-2235, 2001; Reik and Murrell., Nature, vol. 405, page 408-409, 2000; Berger et al., Molecular Cell, vol. 8, pages 263-268). One may also produce a singular integrant by cotransfecting the bacterial RecA protein with or without nuclear localization signal along with sequences that are homologous to the target sequence (e.g., a target endogenous sequence or integrated transgene sequence). Alternatively, a nucleic acid sequence that encodes a RecA protein with nuclear localization signals can be cotransfected (Shibata et al., Proc. Natl. Acad. Sci. U.S.A. 2001 Jul. 17; 98(15):8425-32; Muyrers et al., Trends Biochem. Sci. 2001 May; 26(5):325-31; Paul et al., Mutat. Res. 2001 Jun. 5; 486(1): 11-9; Shcherbakova et al., Mutat. Res. 2000 February 16; 459(1):65-71; Lantsov. Mol. Biol. (Mosk). 1994 May-June; 28(3):485-95). Other methods as taught in U.S. Published Application 2002/0132257 and European Published Application EP1229134, “Use of post-transcriptional gene silencing for identifying nucleic acid sequences that modulate the function of a cell”, are also contemplated as useful applications of the unique dsRNA hairpin constructs and dsRNA expression constructs of the invention.

See also the methods and teaching of published applications WO 00/01846, EP1093526, and EP1197567, “Characterization of Gene Function Using Double-Stranded RNA Inhibition”, incorporated herein by reference, which provides a method of identifying DNA responsible for conferring a particular phenotype in a cell. The method comprises constructing a cDNA or genomic library of the DNA of a cell in a suitable vector in an orientation relative to a promoter(s) capable of initiating transcription of the cDNA or DNA to double stranded (ds) RNA upon binding of an appropriate transcription factor to said promoter(s); introducing the library into one or more cells comprising said transcription factor; and identifying and isolating a particular phenotype of the cell comprising the library and identifying the DNA or cDNA fragment from the library responsible for conferring the phenotype.

See also published applications WO 99/32619 and EP1042462, “Genetic inhibition by Double-Stranded RNA”, which teach methods of identifying gene function in an organism comprising the use of double-stranded RNA to inhibit the activity of a target gene of previously unknown function. High throughput screening methods wherein dsRNAs are produced from gene libraries, e.g., genomic DNA or mRNA (cDNA and eRNA) libraries derived from a target cell or organism.

While less desirable, all of such functional genomics methods may utilize randomized nucleic acid sequences or a given sequence for which the function is not known, as described in, e.g., U.S. Pat. No. 5,639,595, the teaching of which is hereby incorporated by reference.

The dsRNA structures and dsRNA expression constructs of the present invention may be used in methods to identify unknown targets that result in the modulation of a particular phenotype, an alteration of gene expression in a cell, or an alteration in polypeptide biological activity in a cell, using either a library based screening approach or a non-library based approach to identify nucleic acids that induce gene silencing. These methods involve the direct delivery of in vitro transcribed dsRNA or the delivery of a plasmid that direct the cell to make its own dsRNA.

Short dsRNA or a plasmid encoding short dsRNA may also administered in any of the functional genomics applications if desired to inhibit dsRNA-mediated toxicity, as taught in U.S. Ser. No. 60/375,636 filed Apr. 26, 2002 and U.S. Ser. No. 10/425,006 filed Apr. 28, 2003 “Methods for Silencing Genes Without Inducing Toxicity”, C. Pachuk, the teaching of which is incorporated herein by reference. To avoid problems associated with transfection efficiency, plasmids are designed to contain a selectable marker to ensure the survival of only those cells that have taken up plasmid DNA. One group of plasmids directs the synthesis of dsRNA that is transcribed in the cytoplasm, while another group directs the synthesis of dsRNA that is transcribed in the nucleus.

### Identification of Genes Using Differential Gene Expression

Differential gene expression analysis can be used to identify a nucleic acid sequence that modulates the expression of a target nucleic acid in a cell. Alterations in gene expression induced by gene silencing can be monitored in a cell into which a dsRNA has been introduced. For example, differential gene expression can be assayed by comparing nucleic acids expressed in cells into which dsRNA has been introduced to nucleic acids expressed in control cells that were not transfected with dsRNA or that were mock-transfected. Gene array technology can be used in order to simultaneously examine the expression levels of many different nucleic acids. Examples of methods for such expression analysis are described by Marrack et al. (Current Opinions in Immunology 12:206-209, 2000); Harkin (Oncologist 5:501-507, 2000); Pelizzari et al. (Nucleic Acids Res. 28:4577-4581, 2000); and Marx (Science 289:1670-1672, 2000).

### Identification of Genes by Assaying Polypeptide Biological Activity

Novel nucleic acid sequences that modulate the biological activity of a target polypeptide can also be identified by examining polypeptide biological activity. Various polypeptide biological activities can be evaluated to identify novel genes according to the methods of the invention. For example, the expression of a target polypeptide(s) may be examined. Alternatively, the interaction between a target polypeptide(s) and another molecule(s), for example, another polypeptide or a nucleic acid may be assayed. Phosphorylation or glycosylation of a target polypeptide(s) may also be assessed, using standard methods known to those skilled in the art.

Identification of nucleic acid sequences involved in modulating the biological activity of a target polypeptide may be carried out by comparing the polypeptide biological activity of a cell transfected with a dsRNA to a control cell that has not been transfected with a dsRNA or that has been mock-transfected. A cell that has taken up sequences unrelated to a particular polypeptide biological activity will perform in the particular assay in a manner similar to the control cell. A cell experiencing PTGS of a gene involved in the particular polypeptide biological activity will exhibit an altered ability to perform in the biological assay, compared to the control.

### Example 11

### Design and Delivery of Vectors for Intracellular Synthesis of dsRNA

The utilization of dsRNAs may induce even less toxicity or adverse side-effects when dsRNA resides in certain cellular compartments. Therefore, expression plasmids that transcribe candidate and/or short dsRNA in the cytoplasm and in the nucleus may be utilized. There are two classes of nuclear transcription vectors: one that is designed to express polyadenylated dsRNA (for example, a vector containing an RNA polymerase II promoter and a poly A site) and one that expresses non-adenylated dsRNA (for example, a vector containing an RNA polymerase II promoter and no poly A site, or a vector containing a T7 promoter). Different cellular distributions are predicted for the two species of RNA; both vectors are transcribed in the nucleus, but the ultimate destinations of the RNA species are different intracellular locations. Intracellular transcription may also utilize bacteriophage T7 and SP6 RNA polymerase, which may be designed to transcribe in the cytoplasm or in the nucleus. Alternatively, Qbeta replicase RNA-dependent RNA polymerase may be used to amplify dsRNA. Viral RNA polymerases, either DNA and RNA dependent, may also be used. Alternatively, dsRNA replicating polymerases can be used. Cellular polymerases such as RNA Polymerase I, II, or III or mitochondrial RNA polymerase may also be utilized. Both the cytoplasmic and nuclear transcription vectors contain an antibiotic resistance gene to enable selection of cells that have taken up the plasmid. Cloning strategies employ chain reaction cloning (CRC), a one-step method for directional ligation of multiple fragments (Pachuk et al., Gene 243:19-25, 2000). Briefly, the ligations utilize bridge oligonucleotides to align the DNA fragments in a particular order and ligation is catalyzed by a heat-stable DNA ligase, such as Ampligase, available from Epicentre.

Inducible or Repressible Transcription Vectors

If desired, inducible and repressible transcription systems can be used to control the timing of the synthesis of dsRNA. For example, synthesis of candidate dsRNA molecules can be induced after synthesis or administration of short dsRNA which is intended to prevent possible toxic effects due to the candidate dsRNA. Inducible and repressible regulatory systems involve the use of promoter elements that contain sequences that bind prokaryotic or eukaryotic transcription factors upstream of the sequence encoding dsRNA. In addition, these factors also carry protein domains that transactivate or transrepress the RNA polymerase II. The regulatory system also has the ability to bind a small molecule (e.g., a coinducer or a corepressor). The binding of the small molecule to the regulatory protein molecule (e.g., a transcription factor) results in either increased or decreased affinity for the sequence element. Both inducible and repressible systems can be developed using any of the inducer/transcription factor combinations by positioning the binding site appropriately with respect to the promoter sequence. Examples of previously described inducible/repressible systems include lacI, ara, Steroid-RU486, and ecdysone—Rheogene, Lac (Cronin et al. Genes & Development 15: 1506-1517, 2001), ara (Khlebnikov et al., J Bacteriol. 2000 December; 182(24):7029-34), ecdysone (Rheogene, www.rheogene.com), RU48 (steroid, Wang X J, Liefer K M, Tsai S, O'Malley B W, Roop D R., Proc Natl Acad Sci USA. 1999 Jul. 20; 96(15):8483-8), tet promoter (Rendal et al., Hum Gene Ther. 2002 January; 13(2):335-42. and Larnartina et al., Hum Gene Ther. 2002 January; 13(2):199-210), or a promoter disclosed in WO 00/63364, filed Apr. 19, 2000.

Nuclear Transcription Vectors

Nuclear transcription vectors are designed such that the target sequence is flanked on one end by an RNA polymerase II promoter (for example, the HCMV-IE promoter) and on the other end by a different RNA polymerase II promoter (for example, the SCMV promoter). Other promoters that can be used include other RNA polymerase II promoters, an RNA polymerase I promoter, an RNA polymerase III promoter, a mitochondrial RNA polymerase promoter, or a T7 or SP6 promoter in the presence of T7 or SP6 RNA polymerase, respectively, containing a nuclear localization signal. Bacteriophage or viral promoters may also be used. The promoters are regulated transcriptionally (for example, using a tet ON/OFF system (Forster et al., supra; Liu et al., supra; and Gatz, supra) such that they are only active in either the presence of a transcription-inducing agent or upon the removal of a repressor. A single chromosomal integrant is selected for, and transcription is induced in the cell to produce the nuclear dsRNA.

Those vectors containing a promoter recognized by RNA PolI, RNA PolII, or a viral promoter in conjunction with co-expressed proteins that recognize the viral promoter, may also contain optional sequences located between each promoter and the inserted cDNA. These sequences are transcribed and are designed to prevent the possible translation of a transcribed cDNA. For example, the transcribed RNA is synthesized to contain a stable stem-loop structure at the 5′ end to impede ribosome scanning. Alternatively, the exact sequence is irrelevant as long as the length of the sequence is sufficient to be detrimental to translation initiation (e.g., the sequence is 200 nucleotides or longer). The RNA sequences can optionally have sequences that allow polyA addition, intronic sequences, an HIV REV binding sequence, Mason-Pfizer monkey virus constitutive transport element (CTE) (U.S. Pat. No. 5,880,276, filed Apr. 25, 1996), and/or self splicing intronic sequences.

To generate dsRNA, two promoters can be placed on either side of the target sequence, such that the direction of transcription from each promoter is opposing each other. Alternatively, two plasmids can be cotransfected. One of the plasmids is designed to transcribe one strand of the target sequence while the other is designed to transcribe the other strand. Single promoter constructs may be developed such that two units of the target sequence are transcribed in tandem, such that the second unit is in the reverse orientation with respect to the other. Alternate strategies include the use of filler sequences between the tandem target sequences.

Cytoplasmic Transcription Vectors

Cytoplasmic transcription vectors are made according to the following method. This approach involves the transcription of a single stranded RNA template in the nucleus, which is then transported into the cytoplasm where it serves as a template for the transcription of dsRNA molecules. The DNA encoding the ssRNA may be integrated at a single site in the target cell line, thereby ensuring the synthesis of only one species of candidate dsRNA in a cell, each cell expressing a different dsRNA species.

A desirable approach is to use endogenous polymerases such as the mitochondrial polymerase in animal cells or mitochondrial and chloroplast polymerases in plant cells for cytoplasmic and mitochondrial (e.g., chloroplast) expression to make dsRNA in the cytoplasm. These vectors are formed by designing expression constructs that contain mitochondrial or chloroplast promoters upstream of the target sequence. As described above for nuclear transcription vectors, dsRNA can be generated using two such promoters placed on either side of the target sequence, such that the direction of transcription from each promoter is opposing each other. Alternatively, two plasmids can be cotransfected. One of the plasmids is designed to transcribe one strand of the target sequence while the other is designed to transcribe the other strand. Single promoter constructs may be developed such that two units of the target sequence are transcribed in tandem, such that the second unit is in the reverse orientation with respect to the other. Alternate strategies include the use of filler sequences between the tandem target sequences.

Alternatively, cytoplasmic expression of dsRNA is achieved by a single subgenomic promoter opposite in orientation with respect to the nuclear promoter. The nuclear promoter generates one RNA strand that is transported into the cytoplasm, and the singular subgenomic promoter at the 3′ end of the transcript is sufficient to generate its antisense copy by an RNA dependent RNA polymerase to result in a cytoplasmic dsRNA species.

### Example 12

### Non-library Approaches for the Identification of a Nucleic Acid Sequence that Modulates Cell Function. Cellular Gene Expression, or Biological Activity of a Target Polypeptide

Nucleic acid sequences that modulate cell function, gene expression in a cell, or the biological activity of a target polypeptide in a cell may also be identified using non-library based approaches involving PTGS. For example, a single known nucleic acid sequence encoding a polypeptide with unknown function or a single nucleic acid fragment of unknown sequence and/or function can be made into a “candidate” dsRNA molecule. This candidate dsRNA is then transfected into a desired cell type. A short dsRNA or a nucleic acid encoding a short dsRNA is optionally also administered to prevent toxicity. The cell is assayed for modulations in cell function, gene expression of a target nucleic acid in the cell, or the biological activity of a target polypeptide in the cell, using methods described herein. A modulation in cell function, gene expression in the cell, or the biological activity of a target polypeptide in the cell identifies the nucleic acid of the candidate dsRNA as a nucleic acid the modulates the specific cell function, gene expression, or the biological activity of a target polypeptide. As a single candidate dsRNA species is transfected into the cells, the nucleic acid sequence responsible for the modulation is readily identified.

The discovery of novel genes through the methods of the present invention may lead to the generation of novel therapeutics. For example, genes that decrease cell invasion may be used as targets for drug development, such as for the development of cytostatic therapeutics for use in the treatment of cancer. Development of such therapeutics is important because currently available cytotoxic anticancer agents are also toxic for normal rapidly dividing cells. In contrast, a cytostatic agent may only need to check metastatic processes, and by inference, slow cell growth, in order to stabilize the disease. In another example, genes that increase neuronal regeneration may be used to develop therapeutics for the treatment, prevention, or control of a number of neurological diseases, including Alzheimer's disease and Parkinson's disease. Genes that are involved in the ability to support viral replication and be used as targets in anti-viral therapies. Such therapies may be used to treat, prevent, or control viral diseases involving human immunodeficiency virus (HIV), hepatitis C virus (HCV), hepatitis B virus (HBV), and human papillomavirus (HPV). The efficacies of therapeutics targeting the genes identified according to the present invention can be further tested in cell culture assays, as well as in animal models.

### Example 13

### Analysis of RNA from Transfected Cells

Regardless of whether a library based screening approach or a non-library based approach was used to identify nucleic acid sequences, in order to measure the level of dsRNA effector molecule within the cell, as well as the amount of target to mRNA within the cell, a two-step reverse transcription PCR reaction is performed with the ABI PRISM™ 7700 Sequence Detection System. Total RNA is extracted from cells transfected with dsRNA or a plasmid from a dsRNA expression library using Trizol and DNase. Two to three different cDNA synthesis reactions are performed per sample; one for human GAPDH (a housekeeping gene that should be is unaffected by the effector dsRNA), one for the target mRNA, and/or one for the sense strand of the expected dsRNA molecule (effector molecule). Prior to cDNA synthesis of dsRNA sense strands, the RNA sample is treated with T1 RNase. The cDNA reactions are performed in separate tubes using 200 ng of total RNA and primers specific for the relevant RNA molecules. The cDNA products of these reactions are used as templates for subsequent PCR reactions to amplify GAPDH, the target cDNA, and/or the sense strand copied from the dsRNA. All RNA are quantified relative to the internal control, GAPDH.

### Example 14

### Target Sequence Identification

To identify the target sequence affected by a dsRNA, using any of the above-described methods, DNA is extracted from expanded cell lines (or from the transfected cells if using a non-integrating dsRNA system) according to methods well known to the skilled artisan. The dsRNA encoding sequence of each integrant (or non-integrated dsRNA molecule if using a non-library based method) is amplified by PCR using primers containing the sequence mapping to the top strand of the T7 promoter (or any other promoter used to express the dsRNA). Amplified DNA is then cloned into a cloning vector, such as pZERO blunt (Promega Corp.), and then sequenced. Sequences are compared to sequences in GenBank and/or other DNA databases to look for sequence identity or homology using standard computer programs. If the target mRNA remains unknown, the mRNA is cloned from the target cell line using primers derived from the cloned dsRNA by established techniques (Sambrook et al., supra). Target validation is then carried out as described herein.

In the stably integrated dsRNA expression system described above, despite efforts to reduce negative position effects, inefficient dsRNA synthesis by PCR methods may occur. This can be circumvented by rescuing the integrated cDNA or randomized nucleic sequences into replicating plasmids. Rescued plasmids are amenable to amplification in bacteria and to sequencing. Rescue is achieved by re-transfecting the population of cells transfected with the dsRNA expression library with the rescue plasmid and a plasmid encoding Cre recombinase. The rescue plasmid carries a bacterial origin of replication, a bacterial antibiotic selection marker, an SV40 origin of replication, and an SV40 T antigen expression cassette, as well as loxP sites positioned as an inverted repeat to allow Cre-mediated double recombination. The SV40-based origin of replication in the rescue plasmid allows amplification of rescued sequences in the integrated cells. Following rescue, higher levels of transcription are anticipated, thereby favoring dsRNA formation. The cells are then screened for modulations in cell function, target nucleic acid expression, or target polypeptide biological activity changes as described herein.

Summary of Results

We have shown that intracellular expression of dsRNA does not induce the RNA stress response. See e.g., US 2002/0132257 A1, published Sep. 19, 2002, “The use of post-transcriptional gene silencing for identifying nucleic acid sequences that modulate the function of a cell”. The cells that were used in these experiments were competent for RNA stress response induction as was demonstrated by the ability of cationic lipid complexed poly(I)(C) and in vitro transcribed RNA to induce/activate all tested components of this response. In addition, the cells were found to be responsive to exogenously added interferon. These results imply that the cells used for these experiments are not defective in their ability to mount an RNA stress response and therefore can be used as predictors for other cells, both in cell culture and in vivo in animal models. This method, which does not induce the interferon stress response, has also been found to effectively induce PTGS. This method therefore provides a method to induce PTGS without inducing an undesired RNA stress response.

Although these results were generated using a vector that utilizes a T7 transcription system and therefore expresses dsRNA in the cytoplasm, the vector system can be changed to other systems that express dsRNA intracellularly. Similar results are expected with these expression systems. These systems include, but are not limited to, systems that express dsRNA or hairpin RNA molecules in the nucleus, in the nucleus followed by transport of the RNA molecules to the cytoplasm, or in the cytoplasm using non-T7 RNA polymerase based expression systems.

Summary

Current evidence indicates that long dsRNA molecules are processed intracellularly into smaller ds ribo-oligonucleotides of 21-24 base-pairs. These ribo-oligonucleotides, termed small interfering RNA molecules (siRNAs), have been implicated as the dsRNA species that effect PTGS. Desirable embodiments of the present invention use longer dsRNA molecules that can be processed intracellularly into hundreds of different siRNA molecules, many of which should be effective.

An understanding of the factors influencing the induction of this response has previously been complicated by the fact that induction/activation has primarily been studied using poly(I)(C), as a surrogate for dsRNA. Poly(I)(C) however, is heterogeneous with extensive variability not only in length and sequence but in structure as well. As dsRNA is only one of the many structures that poly(I)(C) adopts and because other structured RNA molecules have been shown to induce/activate various components of the RNA stress response pathway (Hartmann, et al., J Biol Chem, 273(6): p. 3236-46, 1998; Mordechai, et al., Virology, 206(2): p. 913-22, 1995)] induction/activation of this pathway cannot be considered to be exclusively mediated by dsRNA. In addition, RNA structure is not the only parameter involved in the Type I interferon stress response induction. Levels of the activating RNA also effect the induction/activation of some of the components in this pathway.

We demonstrate here that induction/activation of the Type I interferon stress response pathway is also mediated by the method used to deliver dsRNA within a cell. Presumably this reflects a requirement that dsRNA be localized within certain sub-cellular compartments or that certain threshold levels of intracellular RNA be realized before the interferon stress response pathway can be induced and activated. We present an intracellular dsRNA expression system that does not induce or activate the RNA stress response pathway in a human cell line competent for both the production of and responsiveness to Type I interferon. Furthermore, expression of dsRNA using this method was shown to be very efficient at inducing sequence specific PTGS. We demonstrate the ability of this method to down-regulate the expression of target genes by 95% for at least one month. Since the ability to sustain silencing for longer periods of time is clearly an advantage for many PTGS applications such as target validation, functional genomics and therapy, the intracellular dsRNA expression detailed here offers yet another advantage over the use of siRNA.

Results

Since our objective was to find a dsRNA delivery or expression system that would not induce the type I interferon response, it was imperative to demonstrate that the cells used for these studies were competent in carrying out this response. The pathway is branched and RNA mediated initiation and activation of the stress response can occur at multiple points in the pathway. RNA can act to elicit the production of alpha and/or beta interferon in most cell types. Early and key events following exposure to these type I interferons include interferon-mediated activation of the Jak-Stat pathway, which involves tyrosine phosphorylation of STAT proteins (STATs). Activated STATs translocate to the nucleus and bind to specific sites in the promoters of IFN-inducible genes thereby effecting transcription of these genes: the expression of which act in concert to push the cell towards an apoptotic or an anti-proliferative state. Double stranded RNA can also activate the pathway in an interferon independent manner, by directly activating STAT and also by directly activating transcription of a number of interferon stimulated genes. Although there are several hundred interferon-stimulated genes, two of the better-characterized interferon stimulated genes products are protein kinase PKR and 2′5′ oligoadenylate synthetase (2′5′-OAS). These gene products are constitutively expressed at some level in their non-active forms in most cell types.

The choice of a cell culture model for developing a dsRNA delivery or expression system was dependent upon the cell's ability to mount a stress response to dsRNA. The model system needed to fulfill at least two criteria: (i) demonstrable RNA stress response to poly(I)(C) stimulation and (ii) responsiveness to exogenously added type I interferon. In addition, the choice of a human derived cell line was desirable, as many PTGS applications are targeted for use in human cells. The cell line initially evaluated was a human rhabdomyosarcoma (RD) cell line.

Comparison of siRNA and Cytoplasmically Expressed Long RNA Induced Silencing

The effectiveness and persistence with which siRNA and cytoplasmically expressed long RNA can induce gene-specific silencing was compared in a transient SEAP (secreted human placental alkaline phosphatase) expression system. RD cells transiently expressing SEAP were transfected either with one of three different SEAP-specific siRNA molecules or with a T7 RNA polymerase expression plasmid in conjunction with a SEAP-specific dsRNA expression. As an internal control for silencing specificity, the cells used in these studies also transiently expressed secreted murine IL-12. Media collected from transfected cells at multiple time points post-transfection was assayed for both SEAP and IL-12. SEAP expression levels were normalized to IL-12 levels.

The three different siRNA molecules evaluated in this study were found to have different efficacies with respect to their ability to silence SEAP expression. siRNA #1013, administered at 1.4 ug per ˜1×10⁶ cells, was the most potent, inhibiting SEAP expression by about 80% for two days. The lower dose of this siRNA was less effective and maximal inhibition of SEAP expression was observed for only one day. siRNA #497 was moderately effective when given in the highest dose of 1.4 ug, resulting in 70% inhibition of SEAP that lasted for one day. When given at the lower dose, this siRNA resulted in a 40% inhibition of SEAP expression that persisted for two days. The least potent of the siRNA molecules, siRNA#1143, resulted in a short-lived 55% inhibition when the siRNA was administered at the highest dose. All siRNA-mediated inhibition was lost between days four and six in these studies, consistent with what has been reported by others (Elbashir, et al., Nature. 411(6836): p. 494-8, 2001) (Proceedings of the Keystone Symposia: RNA Interference, Cosuppression and Related Phenomena, Taos, N. Mex., February 2002).

In contrast, the use of the T7 cytoplasmic transcription system to generate long dsRNA in the cytoplasm of transfected cells was found to induce an 85% inhibition of SEAP expression that persisted for the length of the experiment, ten days. A dose response experiment in which different amounts of the dsRNA expression vector were transfected into cells indicates that the level of inhibition correlates with the amount of SEAP-specific dsRNA. A control dsRNA expression vector encoding an irrelevant dsRNA had no effect on SEAP expression. The observed long-lived silencing response in this experiment and in the PSA silencing experiment is consistent with the continued expression of dsRNA that this expression system affords.

Similar studies have been repeated by us in other mammalian cell lines, indicating that this method is a generic silencing method that can be used in a variety of systems.

Summary

An efficient method for inducing long-term gene silencing in mammalian systems has been identified. This method allows for the sustained expression of dsRNA (e.g., long dsRNA) within cells (e.g., vertebrate cells, such as mammalian cells) without invoking the components of the RNA stress or type I interferon response pathway.

We have shown that cytoplasmic expression of long dsRNA does not invoke an RNA stress response and is a very potent inducer of gene silencing. For administration of dsRNA (e.g., long dsRNA), delivery systems other than cationic lipids are desirable. These other delivery systems, such as those described herein, may also prevent an interferon response. Additionally, short dsRNA can be administered to inhibit dsRNA-mediated toxicity as described herein.

### Example 15

### Optimization of the Concentrations and Relative Ratios of In Vitro or in Vivo Produced dsRNA and Delivery Agent

If desired, optimal concentrations and ratios of dsRNA to a delivery agent such as a cationic lipid, cationic surfactant, or local anesthetic can be readily determined to achieve low toxicity and to efficiently induce gene silencing using in vitro or in vivo produced dsRNA.

Summary of Factors Effecting Nucleic Acid/Cationic Lipid Interactions

Cationic lipid DNA interactions are electrostatic. Electrostatic interactions are highly influenced by the ionic components of the medium. The ability to form stable complexes is also dependent upon the intermolecular interactions between the lipid molecules. At low concentrations, certain inter-lipid interactions are preferred; at higher lipid concentrations, rapid condensates are formed due to higher order interactions. Although local interactions are similar in both of these instances (e.g., phosphoryl groups in the DNA and the charged cationic head group), the long range and inter-lipid interactions are substantially different. Similarly, structurally diverse variants can be obtained simply by changing the charge ratio of the complex by mixing varying amounts of cationic lipid with fixed concentrations of the nucleic acid or vice versa. This variation in the structure of the complexes is evidenced by altered physical properties of the complexes (e.g., differences in octanol partitioning, mobility on density gradients, charge density of the particle, particle size, and transfectability of cells in culture and in vivo) (Pachuk et al. DNA Vaccines—Challenges in Delivery, Current Opinion in Molecular Therapeutics, 2(2) 188-198, 2000 and Pachuk et al., BBA, 1468, 20-30, (2000)). Furthermore, different lipids, local anesthetics, and surfactants differ in their interactions between themselves, and therefore novel complexes can be formed with differing biophysical properties by using different lipids singularly or in combination. For each cell type, the following titration can be carried out to determine the optimal ratio and concentrations that result in complexes that do not induce the stress response or interferon response. At several of these concentrations PTGS is predicted to be induced; however, PTGS is most readily observed under conditions that result in highly diminished cytotoxicity.

Complex Formation

dsRNA is either produced by in vitro transcription using the T7 promoter and polymerase or another RNA polymerase, such as an E. coli RNA polymerase. dsRNA can also be produced in an organism or cell using endogenous polymerases.

Concentrations of dsRNA, such as PSA-specific dsRNA, are varied from 1 pg to 10 μg. In some instances, 150 ng of a plasmid that encodes a reporter of interest (PSA) to be silenced may be comixed at a concentration between 10 ng and 10 μg. The concentration of cationic lipid, cationic surfactant, local anesthetic, or any other transfection facilitating agent that interacts with the nucleic acid electrostatically are varied at each of the dsRNA concentrations to yield charge ratios of 0.1 to 1000 (positive/negative) (i.e., the ratio of positive charge from lipids or other delivery agents to negative charge from DNA or RNA). The complexes are prepared in water or in buffer (e.g., phosphate, HEPES, citrate, Tris-HCl, Tris-glycine, malate, etc. at pH values that range from 4.0 to 8.5), may contain salt (e.g., 1-250 mM), and may contain glycerol, sucrose, trehalose, xylose, or other sugars (e.g., mono-, di-, or polysaccharide). The mixture is allowed to sit at room temperature, desirably for 30 minutes, and may be stored indefinitely. The complexes are premixed in serum free media. The nucleic acid and the transfecting reagent may be mixed either through direct addition or through a slow mixing process, such as across a dialyzing membrane or through the use of a microporous particle or a device that brings the two solutions together at a slow rate and at low concentrations. In some instances, the two interacting components are mixed at low concentrations, and the final complex is concentrated using a diafilteration or any other concentrating device. Alternatively, if the complexes are formed at high concentrations of either or both of the interacting components, the complexes may be diluted to form an ideal transfection mixture.

Transfection Protocol and Analysis of dsRNA Stress Response

Complexes are added to cells that are ˜60-80% confluent in serum free media. The complexes are incubated for various times (e.g., 10 minutes to 24 hours) with the cells at 37° C. and diluted with serum containing media or washed and replated in serum free media. The cells are monitored for toxicity and analyzed at various times for signs of dsRNA response (e.g., TUNEL assay to detect nicked DNA, phosphorylation of EIF2alpha, induction and activation of 2′5′ OAS, or interferon-alpha and -beta). Transfection conditions that result in less than 50%, 25%, 10%, or 1% cytotoxicity or that result in a less than 20, 10, 5, 2, or 1.5-fold induction of a stress response are analyzed to determine if PTGS was efficiently induced.

Determining Induction of PTGS

PSA protein levels are determined in cell culture media using standard methods. The data is normalized to the number of live cells in culture to determine the concentrations required to induce PTGS.

Results

Using the above method, cationic lipid complexes of dsRNA induced toxicity at certain ranges. With lipofectamine as the cationic lipid, positive to negative charge ratios greater than 10 did not produce any detectable toxicity at any of the concentrations of dsRNA tested and induced a high level of PTGS, resulting in highly decreased levels of PSA in the culture medium. The RNA concentration ranges tested were 1 pg to 100 ng with a constant amount of lipofectamine (10 uL of a 2 mg/mL solution from GIBCO-BRL Life Technologies, Bethesda, Md.).

Ratio of Target Gene-Specific dsRNA to Short dsRNA

If desired, the above method can be used to optimize the ratio of short sdRNA that inhibits toxicity to target gene-specific dsRNA that silences the target gene. For example, concentrations of dsRNA specific for a target gene, such as PSA-specific dsRNA, are varied from 50 ng to 5 ug per one million cells, and concentrations of short dsRNA (e.g., random short dsRNA molecules used to inhibit toxicity) are varied from 50 ng to 5 ug per one million cells. The ratio of the number of moles of short dsRNA to moles of target-specific dsRNA to is varied from 1000:1, 1:1, to 1:25. In some instances, 150 ng of a plasmid that encodes a reporter of interest (PSA) to be silenced may be comixed at a concentration between 10 ng and 10 μg. The concentration of cationic lipid, cationic surfactant, local anesthetic, or any other transfection facilitating agent that interacts with the nucleic acid electrostatically are varied at each of the dsRNA concentrations to yield charge ratios of 0.1 to 1000 (positive/negative) (i.e., the ratio of positive charge from lipids or other delivery agents to negative charge from DNA or RNA). The complexes are prepared and tested as described above

Applications of Present Methods

Short dsRNA molecules can be used in conjunction with exogenously added or endogenously expressed dsRNA molecules in gene silencing applications to prevent the activation of PKR that would otherwise be elicited by the latter dsRNA. Currently, the administration of such exogenously added dsRNA to cells and animals for gene-silencing experiments is limited by the cytotoxicity induced by dsRNA (e.g., long dsRNA). Short dsRNA or a vector stably or transiently expressing short dsRNA can be delivered before (e.g., 10, 20, 30, 45, 60, 90, 120, 240, or 300 minutes before), during, or after (e.g., 2, 5, 10, 20, 30, 45, 60, or 90 minutes after) the delivery of exogenous dsRNA or a vector encoding dsRNA to animals or cell cultures. A vector expressing a short dsRNA can also be administered up to 1, 2, 3, 5, 10, or more days before administration of dsRNA homologous to a target nucleic acid. A vector expressing short dsRNA can be administered any number of days before the administration of dsRNA homologous to a target nucleic acid (e.g., target-specific dsRNA) or a vector encoding this dsRNA, as long as the dsRNA-mediated stress response pathway is still inhibited by the short dsRNA when the target-specific dsRNA is administered. The timing of the delivery of these nucleic acids can be readily be selected or optimized by one skilled in the art of pharmacology using standard methods. See also the teaching of U.S. Ser. No. 60/375,636 filed Apr. 26, 2002 and U.S. Ser. No. 10/425,006 filed Apr. 28, 2003, “Methods for Silencing Genes Without Inducing Toxicity”, C. Pachuk, which is incorporated herein by reference.

### Example 16

### Exemplary Clinical and Industrial Applications of the Constructs and Methods of the Invention

The dsRNA structures, e.g., partial and/or forced hairpins, and dsRNA expression constructs of the invention can also be used in methods to treat, stabilize, or prevent diseases associated with the presence of an endogenous or pathogen protein in vertebrate organisms (e.g., human and non-human mammals). These methods are expected to be especially useful for therapeutic treatment for viral diseases, including chronic viral infections such as HBV, HIV, papilloma viruses, and herpes viruses. In some embodiments, the methods of the invention are used to prevent or treat acute or chronic viral diseases by targeting a viral nucleic acid necessary for replication and/or pathogenesis of the virus in a mammalian cell. Slow virus infection characterized by a long incubation or a prolonged disease course are especially appropriate targets for the methods of the invention, including such chronic viral infections as HTLV-I, HTLV-II, EBV, HBV, CMV, HCV, HIV, papilloma viruses, and herpes viruses. For prophylaxis of viral infection, the selected gene target is desirably introduced into a cell together with the short dsRNA and long dsRNA molecules of the invention. Particularly suitable for such treatment are various species of the Retroviruses, Herpesviruses, Hepadnaviruses, Poxviruses, Papillomaviruses, and Papovaviruses. Exemplary target genes necessary for replication and/or pathogenesis of the virus in an infected vertebrate (e.g., mammalian) cell include nucleic acids of the pathogen or host necessary for entry of the pathogen into the host (e.g., host T cell CD4 receptors), nucleic acids encoding proteins necessary for viral propagation (e.g., HIV gag, env, and pol), and regulatory genes such as tat and rev. Other exemplary targets include nucleic acids for HIV reverse transcriptase, HIV protease, HPV6 L1 and E2 genes, HPV 11 L1 and E2 genes, HPV16 E6 and E7 genes, HPV18 E6 and E7 genes, HBV surface antigens, core antigen, and reverse transcriptase, HSD gD gene, HSVvp16 gene, HSVgC, gH, gL, and gB genes, HSV ICP0, ICP4, and ICP6 genes; Varicella zoster gB, gC and gH genes, and non-coding viral polynucleotide sequences which provide regulatory functions necessary for transfer of the infection from cell to cell (e.g., HIV LTR and other viral promoter sequences such as HSV vp16 promoter, HSV-ICP0 promoter, HSV-ICP4, ICP6, and gD promoters, HBV surface antigen promoter, and HBV pre-genomic promoter). Desirably, a dsRNA (e.g., long dsRNA) of the invention reduces or inhibits the function of a viral nucleic acid in the cells of a mammal or vertebrate, and a short dsRNA of the invention blocks the dsRNA stress response that may be triggered by dsRNA.

Exemplary retroviral targets include, but are not limited to, HIV-1 and 2, (LTR promoter element) which drives the expression of most or all of the HIV genes gag, integrase, pol, env, vpx, vpr, vif, nef, HTLV-1 and 2, and pro. Exemplary Hepatitis B the promoters include promoters for antigen genes, for core and e antigen, polymerase, and X protein. Exemplary Hepatitis B target genes include genes encoding surface antigen, core and antigen, polymerase, and X protein.

Exemplary Pox viruses include small pox and vaccinia. Some examples of genes and their promoters are the early, intermediate, and late stage promoters; and promoters and coding sequences for RNA polymerase (multi-subunit), Early transcription factor, poly(A) polymerase, capping enzyme, RNA methyltransferase, DNA-dependent ATPase, RNA/DNA-dependent NTPase, DNA topoisomerase I, nicking-joining enzyme, protein kinase 1 and 2, glutaredoxin, C23L-secreted protein, core proteins, virion proteins, membrane proteins and glycoproteins, transcactivators, DNA polymerase, and complement inhibitor.

Exemplary Herpesviruses include HSV-1 and 2, CMV, EBV, and chicken pox. Exemplary promoters for these viruses include the immediate early, early, intermediate and late promoters, and exemplary genes include any gene expressed from these promoters such as those encoding the immediate early proteins including ICP0, ICP4 and ICP6, vp16, capsid proteins, virion proteins, tegument proteins, envelope proteins and glycoproteins including gD and gB, helicase/primase, DNA polymerase, matrix protein, regulatory proteins, protein kinase, and other proteins.

Examples of Human Papillomaviruses include types 1, 2, 3, 4, 5, 6, 8, 11, 13, 16, 18, 31, 33, 35, 39, 41, 42, 47, 51, 57, 58, 63, and 65. Exemplary promoters of interest are those that drive the expression of E6 and E7, E1, E2, E3 and E4 and E5, and L1, and L2, and exemplary genes include the aforementioned genes.

Examples of adenoviral promoters and genes include promoters and coding sequences for E1A, E2A, E4, E2B-TP, E2 Bpol, Iva2, L1-L5, E1B genes, and E3 genes.

Other exemplary viral promoters and genes include promoters and genes of any of the following viruses: parvoviruses, Encephalitic viruses such as West Nile and Japanese encephalitis, Dengue, Yellow fever, Ebola, Marburg, polio, measles, mumps, as well as other viruses in the families of picornaviridae, calciviridae, astroviridae, togaviridae, flaviviridae, coronaviridae, rhabdoviridae, filoviridae, paramyxoviridae, orthomyxoviridae, bunyaviridae, arenaviridae, and reoviridae.

Other exemplary pathogens include bacteria, rickettsia, chlamydia, fungi, and protozoa such as extraintestinal pathogenic protozoa which cause malaria, babesiosis, trypanosomiasis, leishmaniasis, or toxoplasmosis. The intracellular malaria-causing pathogen Plasmodium species P. falciparum, P. vivax, P. ovale, and P. malariae are desirable targets for dsRNA-mediated gene silencing, especially in the chronic, relapsing forms of malaria. Other intracellular pathogens include Babesia microti and other agents of Babesiosis, protozoa of the genus Trypanosoma that cause African sleeping sickness and American Trypanosomiasis or Chagas' Disease; Toxoplasma gondii which causes toxoplasmosis, Mycobacterium tuberculosis, M. bovis, and M avium complex which cause various tuberculous diseases in humans and other animals. Desirably, a dsRNA (e.g., long dsRNA) of the invention reduces or inhibits the function of a pathogen nucleic acid in the cells of a mammal or vertebrate, and a short dsRNA of the invention blocks the dsRNA stress response that may be triggered by dsRNA.

In some methods for the prevention of an infection, a pathogen target gene or a region from a pathogen target gene (e.g., a region from an intron, exon, untranslated region, promoter, or coding region) is introduced into the cell or animal. For example, this target nucleic acid can be inserted into a vector that desirably integrates in the genome of a cell and administered to the cell or animal. Alternatively, this target nucleic acid can be administered without being incorporated into a vector. The presence of a region or an entire target nucleic acid in the cell or animal is expected to enhance the amplification of the simultaneously or sequentially administered dsRNA that is homologous to the target gene. The amplified dsRNA or amplified cleavage products from the dsRNA silence the target gene in pathogens that later infect the cell or animal. Short dsRNA is also administered to the cell or animal to inhibit dsRNA-mediated toxicity.

Similarly, to silence an endogenous target gene that is not currently being expressed in a particular cell or animal, it may be necessary to introduce a region from the target gene into the cell or animal to enhance the amplification of the administered dsRNA that is homologous to the target gene. The amplified dsRNA or amplified cleavage products from the dsRNA desirably prevent or inhibit the later expression of the target gene in the cell or animal. Desirably, short dsRNA is also administered to inhibit toxic effects.

Still other exemplary target nucleic acids encode a prion, such as the protein associated with the transmissible spongiform encephalopathies, including scrapie in sheep and goats; bovine spongiform encephalopathy (BSE) or “Mad Cow Disease”, and other prion diseases of animals, such as transmissible mink encephalopathy, chronic wasting disease of mule deer and elk, and feline spongiform encephalopathy. Prion diseases in humans include Creutzfeldt-Jakob disease, kuru, Gerstmann-Straussler-Scheinker disease (which is manifest as ataxia and other signs of damage to the cerebellum), and fatal familial insomnia. Desirably, a dsRNA (e.g., long dsRNA) of the invention reduces or inhibits the function of a prion nucleic acid in the cells of a mammal or vertebrate, and a short dsRNA of the invention blocks the dsRNA stress response that may be triggered by dsRNA.

The invention also provides compositions and methods for treatment or prophylaxis of a cancer in a mammal by administering to the mammal one or more of the compositions of the invention in which the target nucleic acid is an abnormal or abnormally expressed cancer-causing gene, tumor antigen or portion thereof, or a regulatory sequence. Desirably, the target nucleic acid is required for the maintenance of the tumor in the mammal. Exemplary oncogene targets include ABL1, BRAF, BCL1, BCL2, BCL6, CBFA2, CSF1R, EGFR, BRBB2 (HER-2/neu), FOS, HRAS, MYB, MYC, LCK, MYCL1, MYCN, NRAS, ROS1, RET, SRC, and TCF3. Such an abnormal nucleic acid can be, for example, a fusion of two normal genes, and the target sequence can be the sequence which spans that fusion, e.g., the bcr/abl gene sequence (Philadelphia chromosome) characteristic of certain chronic myeloid leukemias, rather than the normal sequences of the non-fused bcr and abl (see, e.g., WO 94/13793, published Jun. 23, 1994, the teaching of which is hereby incorporated by reference). Viral-induced cancers are particularly appropriate for application of the compositions and methods of the invention. Examples of these cancers include human-papillomavirus (HPV) associated malignancies which may be related to the effects of oncoproteins, E6 and E7 from HPV subtypes 16 and 18, p53 and RB tumor suppressor genes, and Epstein-Barr virus (EBV) which has been detected in most Burkitt's-like lymphomas and almost all HIV-associated CNS lymphomas. The composition is administered in an amount sufficient to reduce or inhibit the function of the tumor-maintaining nucleic acid in the mammal.

The gene silencing methods of the present invention may also employ a multitarget or polyepitope approach. Desirably, the sequence of the dsRNA includes regions homologous to genes of one or more pathogens, multiple genes or epitopes from a single pathogen, multiple endogenous genes to be silenced, or multiple regions from the same gene to be silenced. Exemplary regions of homology including regions homologous to exons, introns, or regulatory elements such as promoter regions and non-translated regions.

The methods of the invention may also be useful in any circumstances in which PKR suppression is desired; e.g., in DNA expression systems in which small amounts of dsRNA may be inadvertently formed when transcription occurs from cryptic promoters within the non-template strand. The present invention is also useful for industrial applications such as the manufacture of dsRNA molecules in vertebrate cell cultures. The present invention can be used to make “knockout” or “knockdown” vertebrate cell lines or research organisms (e.g., mice, rabbits, sheep, or cows) in which one or more target nucleic acids are silenced. The present invention also allows the identification of the function of a gene by determining the effect of inactivating the gene in a vertebrate cell or organism. These gene silencing methods can also be used to validate a selected gene as a potential target for drug discovery or development.

### Other Embodiments

From the foregoing description, it will be apparent that variations and modifications may be made to the invention described herein to adopt it to various usages and conditions. Such embodiments are also within the scope of the following claims.

All publication, patents, and patent applications mentioned in this specification are herein incorporated by reference to the same extent as if each individual publication, patent, or patent application was specifically and individually indicated to be incorporated by reference.

What is claimed is:

1. A nucleic acid molecule that comprises or that encodes, in 5′ to 3′ order, a first region of interest, a first base-paired region, a loop region, and a second base-paired region, wherein said first and second base-paired regions are capable of base-pairing to each other or are base-paired to each other.

2. The nucleic acid molecule of claim 1, further comprising a second region of interest downstream of said second base-paired region, wherein said first and second regions of interest are capable of base-pairing to each other or are based-paired to each other.

3. (canceled)

4. The nucleic acid molecule of claim 2, wherein said first region of interest has substantial identity to a region of a target gene and said second region of interest has substantial complementarity to said target gene, and wherein said nucleic acid molecule inhibits expression of said target gene in a cell.

5. The nucleic acid molecule of claim 4, wherein said first region of interest has substantial identity to a region of two or more target genes, and said second region of interest has substantial complementarity to said region of said two or more target genes, and wherein said nucleic acid molecule inhibits expression of said two or more target genes in a cell.

6. The nucleic acid molecule of claim 1, wherein said nucleic acid molecule comprises deoxyribonucleotides, ribonucleotides, or a mixture thereof.

7. The nucleic acid molecule of claim 4, wherein said target gene is a nucleic acid molecule associated with a disease or disorder, a bacterial infection, a viral infection, a yeast infection, or double-stranded ribonucleic acid (dsRNA)-mediated toxicity, or encodes a bacterial polypeptide, a viral polypeptide, a yeast polypeptide, a polypeptide associated with a disease or disorder, or a polypeptide associated with double-stranded ribonucleic acid (dsRNA)-mediated toxicity.

8. (canceled)

9. (canceled)

10. The nucleic acid molecule of claim 1, wherein said first region of interest is at least 1 to 1000 nucleotides.

11-15. (canceled)

16. The nucleic acid molecule of claim 10, wherein said first region of interest is at least 19 to 26 nucleotides.

17. (canceled)

18. (canceled)

19. The nucleic acid molecule of claim 2, wherein said second region of interest is at least 1 to 1000 nucleotides.

20-24. (canceled)

25. The nucleic acid molecule of claim 19, wherein said second region of interest is at least 19 to 26 nucleotides.

26-53. (canceled)

54. The nucleic acid molecule of claim 1, wherein said loop region is at least 5 to 15 nucleotides.

55. A pharmaceutical composition comprising the nucleic acid molecule of claim 1 and a pharmaceutically acceptable carrier or diluent.

56. A pharmaceutical composition comprising a vector construct, said construct comprising, at the 5′ end, a promoter that is operably linked to a nucleic acid molecule encoding the nucleic acid molecule of claim 1, wherein transcription of said nucleic acid molecule produces a RNA hairpin.

57. A method for generating an RNA hairpin comprising transcribing a nucleic acid molecule of claim 1, wherein transcription of said nucleic acid molecule produces a RNA hairpin.

58. The method of claim 57, wherein said nucleic acid molecule further encodes a second region of interest downstream of said second base-paired region, wherein said first and second regions of interest are base-paired to each other.

59. The method of claim 58, wherein the 5′ end of said RNA hairpin comprising the first region of interest and the 3′ end of said RNA hairpin comprising the second region of interest are base-paired and partially overlap to form a partial RNA hairpin having a non-overlapping region, wherein the 5′ end of said first region of interest extends beyond the 3′ end of said second region of interest.

60. The method of claim 58, wherein the 5′ end of said RNA hairpin comprising the first region of interest and the 3′ end of said RNA hairpin comprising the second region of interest are base-paired and partially overlap to form a partial RNA hairpin having a non-overlapping region, wherein the 3′ end of said second region of interest extends beyond the 5′ end of said first region of interest.

61. The method of claim 59, wherein said non-overlapping region of said partial RNA hairpin is extended in vivo by an RNA-dependent RNA polymerase.

62. The method of claim 61, wherein said RNA-dependent RNA polymerase is endogenous to said host cell.

63. The method of claim 61, wherein said RNA-dependent RNA polymerase is exogenous to said host cell and is provided to said host cell.

64. A method for inhibiting the expression of a target gene in a cell, said method comprising administering to a subject in need thereof, the nucleic acid molecule of claim 2, wherein said administering inhibits or reduces expression of a target gene, relative to expression of said target gene in a subject not administered said nucleic acid molecule.

65. (canceled)

66. The method of claim 64, wherein the 5′ end of said RNA hairpin comprising the first region of interest and the 3′ end of said RNA hairpin comprising the second region of interest are base-paired and partially overlap to form a partial RNA hairpin having a non-overlapping region, wherein the 5′ end of said first region of interest extends beyond the 3′ end of said second region of interest.

67. The method of claim 64, wherein the 5′ end of said RNA hairpin comprising the first region of interest and the 3′ end of said RNA hairpin comprising the second region of interest are base-paired and partially overlap to form a partial RNA hairpin having a non-overlapping region, wherein the 3′ end of said second region of interest extends beyond the 5′ end of said first region of interest.

68. The method of claim 66, wherein said non-overlapping region of said partial RNA hairpin is extended in vivo by an RNA-dependent RNA polymerase.

69. The method of claim 68, wherein said RNA-dependent RNA polymerase is endogenous to said host cell.

70. The method of claim 68, wherein said RNA-dependent RNA polymerase is exogenous to said host cell and is provided to said host cell.

71. The method of claim 64, wherein said first region of interest has substantial identity to a region of said target gene and said second region of interest has substantial complementarity to said target gene, and wherein said nucleic acid molecule inhibits expression of said target gene in a cell of said subject.

72. The method of claim 71, wherein said first region of interest has substantial identity to a region of two or more target genes, and said second region of interest has substantial complementarity to said region of said two or more target genes, and wherein said nucleic acid molecule inhibits expression of said two or more target genes in a cell of said subject.

73. The method of claim 71, wherein said target gene is a nucleic acid molecule associated with a disease or disorder, a bacterial infection, a viral infection, a yeast infection, or double-stranded ribonucleic acid (dsRNA)-mediated toxicity, or encodes a bacterial polypeptide, a viral polypeptide, a yeast polypeptide, a polypeptide associated with a disease or disorder, or a polypeptide associated with double-stranded ribonucleic acid (dsRNA)-mediated toxicity.

74-116. (canceled)
